(19) United States
(12) Patent Application Publication
(10) Pub. No.: US 2023/0062996 A1
(43) Pub. Date: Mar. 2, 2023

(54) INTRANASAL DRUG DELIVERY DEVICE, SYSTEM, AND PROCESS

(71) Applicant: Rocket Science Health Corp., Vancouver (CA)

(72) Inventors: James Patrick JACKSON, Victoria (CA); Aaron Olafur Laurence PHILIPPSEN, Victoria (CA); Joshua Adrian COUTTS, Victoria (CA); Wesley Barrett CHAMBERLIN, Victoria (CA); Hannah Czaja RUSAK-GILLRIE, Victoria (CA); Iman NIKNIA, Victoria (CA); Julian Snyder GROVE, Victoria (CA); Nicholas David ALLAN, Victoria (CA); David James ALT, North Vancouver (CA); Kenneth Colin MacNarin IRVING, Victoria (CA); Peter OXLEY, Rothesay (CA); Kenza Elizabeth COUBROUGH, Phoenix, AZ (US); Evan MCCORDICK, Rye, NY (US)

(21) Appl. No.: 17/768,188

(22) PCT Filed: Oct. 9, 2020
(86) PCT No.: PCT/IB2020/000849

§ 371 Date: Apr. 11, 2022

Related U.S. Application Data
(60) Provisional application No. 62/914,180, filed on Oct. 11, 2019.
(60) Provisional application No. 62/941,202, filed on Nov. 27, 2019.
(60) Provisional application No. 62/914,070, filed on Oct. 11, 2019.
(60) Provisional application No. 62/914,361, filed on Oct. 11, 2019.
(60) Provisional application No. 62/914,161, filed on Oct. 11, 2019.

(51) Int. Cl. A61B 1/233; A61M 15/00

(52) U.S. Cl. CPC A61B 1/233; A61M 15/009

(57) ABSTRACT

Provided herein are intranasal fluid delivery devices and apparatus comprising a dispensing tip a shot chamber carrying a fluid, the shot chamber having a diaphragm at one end and a plunger at the other end, and an actuator connected to a push rod moveable toward the shot chamber.

### CROSS-REFERENCE

This application claims priority to U.S. Provisional Application No. 62/914,070, filed Oct. 11, 2019, U.S. Provisional Application No. 62/914,161, filed Oct. 11, 2019, U.S. Provisional Application No. 62/914,180, filed Oct. 11, 2019, U.S. Provisional Application No. 62/914,361, filed Oct. 11, 2019, and U.S. Provisional Application No. 62/914,202, filed Oct. 11, 2019, all of which are incorporated by reference herein in its entirety.

### FIELD

The present disclosure generally relates to the field of drug delivery and intranasal devices.

### BACKGROUND

Intranasal drug delivery provides an alternative means for drug administration by a subject as compared to other forms such as intravenous or oral. The nasal cavity of a subject comprises anatomical features that intranasal drug delivery must overcome for effective drug administration.

The inventors have determined a need for improved intranasal delivery devices.

### SUMMARY

In accordance with an aspect, there is provided an intranasal drug delivery device having compliant or flexible, soft nib to precisely locate the dosage and provide comfort for user. The term drug can also be used herein to refer to other agents such as vitamins, fragrance, saline or non-pharmaceutical agents.

Disclosed herein, in some embodiments, is an intranasal fluid delivery device comprising: a) a dispensing tip comprising a flexible nib configured to comply with or conform to a surface of a subject's nasal cavity, thereby enabling a fluid delivery orifice of the dispensing tip to be directed towards an olfactory region of the subject; b) a shot chamber for carrying a fluid, the shot chamber fluidly coupled to the dispensing tip; and c) a plunger configured to drive the fluid from the shot chamber and through the dispensing tip, thereby delivering the fluid from the fluid delivery orifice of the dispensing tip. Disclosed herein, in some embodiments, is an intranasal fluid delivery device comprising: a dispensing tip comprising a flexible nib configured to comply with or conform to a surface of a subject's nasal cavity, thereby enabling a fluid delivery orifice of the dispensing tip to be directed towards an olfactory region of the subject for delivering a fluid therein.

In some embodiments, for any intranasal delivery device described herein, the dispensing tip is configured to deliver the fluid as a liquid jet or a liquid stream. In some embodiments, the liquid jet is a laminar flow or the liquid stream is a laminar flow. In some embodiments, the dispensing tip is configured to deliver the fluid as a spray, mist, or aerosol. In some embodiments, the fluid comprises a powder. In some embodiments, the dispensing tip comprises an atomizer. In some embodiments, the dispensing tip comprises a cannula. In some embodiments, the dispensing tip is tubular. In some embodiments, the dispensing tip has an inner diameter from about 0.3 mm to about 1.5 mm. In some embodiments, the flexible nib comprises a polymer. In some embodiments, the flexible nib comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof. In some embodiments, the dispensing tip comprises a distal portion having a first rigidity and a proximal portion having a second rigidity, and wherein the first rigidity is less than the second rigidity. In some embodiments, the distal portion comprising a first rigidity comprises a portion of the dispensing tip that is about 1 mm to about 15 mm from the fluid delivery orifice. In some embodiments, the dispensing tip comprises a distal portion that is softer than a proximal portion. In some embodiments, the distal portion that is softer than a proximal portion comprises a portion of the dispensing tip that is about 1 mm to about 15 mm from the fluid delivery orifice. In some embodiments, the dispensing tip comprises a distal portion having a first outer diameter and a proximal portion having a second outer diameter, and wherein the first outer diameter is less than the second outer diameter. In some embodiments, the dispensing tip further comprises a nose cushion to limit over-insertion of the dispensing tip within the nasal cavity, wherein the nose cushion configured to provide subject comfort. In some embodiments, the dispensing tip comprises a distal portion having a first outer diameter and a proximal portion having a second outer diameter, and wherein the first outer diameter is greater than the second outer diameter. In some embodiments, the dispensing tip is configured to be inserted to an insertion depth of about 10 mm to about 90 mm within the subject's nasal cavity. In some embodiments, the dispensing tip is configured to be inserted to an insertion depth of about 40 mm to about 70 mm within the subject's nasal cavity. In some embodiments, the fluid delivery orifice of the dispensing tip is configured to be positioned at or near the anterior entry to the olfactory region. In some embodiments, the fluid delivery orifice of the dispensing tip is configured to be positioned in the upper nares of a subject. In some embodiments, the fluid delivery orifice of the dispensing tip is configured to be positioned within about 1 mm to about 25 mm from the anterior entry to the olfactory region.

In some embodiments, for any intranasal delivery device described herein, the dispensing tip comprises a hydrophilic coating applied to an outer surface of the dispensing tip. In some embodiments, the hydrophilic coating is activated by contact with a hydrating medium. In some embodiments, the hydrating medium is water, a gel, a lubricating gel, a viscous liquid, a vapor, or any combination thereof. In some embodiments, the water is water vapor. In some embodiments, activating the hydrophilic coating reduces a surface friction of the hydrophilic coating. In some embodiments, the fluid comprises a pharmaceutical agent or a medicament. In some embodiments, the fluid comprises ketamine or insulin. In some embodiments, the pharmaceutical agent or the medicament is configured to be transported to the central nervous system (CNS) from the olfactory region and at least partly through olfactory neuronal pathways. In some embodiments, the fluid comprises vitamins, fragrance, saline or non-pharmaceutical agents.

In some embodiments, for any intranasal delivery device described herein, the surface of a subject's nasal cavity comprises anatomical features of a nasal cavity of the subject. In some embodiments, the anatomical features comprise nasal turbinates, a nasal valve, or combinations thereof. In some embodiments, the anatomical features comprise a septum of the subject. In some embodiments, the anatomical features comprise an anterior aspect of the nasal passage.

In some embodiments, for any intranasal delivery device described herein, the dispensing tip has an elliptical cross section. In some embodiments, the dispensing tip has a distal portion and a proximal portion, and wherein a first center axis of the distal portion and a second center axis of the proximal portion are non-colinear. In some embodiments, the dispensing tip comprises an off-center drug dispensing channel. In some embodiments, the dispensing tip comprises a protruding element. In some embodiments, the dispensing tip comprises an inflatable balloon surrounding at least a part of a distal portion of the dispensing tip. In some embodiments, the inflatable balloon further surrounds at least a part of a proximal portion of the dispensing tip. In some embodiments, a distal portion of the dispensing tip is curved. In some embodiments, the dispensing tip has a perforation. In some embodiments, the dispensing tip has a perforation on a distal portion of the dispensing tip. In some embodiments, the perforation is on a single side of the dispensing tip. In some embodiments, a distal portion of the dispensing tip has a spiral shape. In some embodiments, exerting the pressure on fluid located within the dispensing tip enables an unraveling of the spiral shape. In some embodiments, the shot chamber is removable.

In some embodiments, for any intranasal fluid delivery device described herein, further comprising a damping mechanism configured to generate a controlled velocity profile of the fluid delivered from the dispensing tip. In some embodiments, the fluid is delivered from the dispensing tip with a velocity of about 0.5 m/s to about 15 m/s. In some embodiments, the fluid is delivered from the dispensing tip with a velocity of about 1.5 m/s to about 9 m/s. In some embodiments, the damping mechanism comprises at least one of a magnet, a spring, a viscous dampener, a sealed chamber with an airflow restriction, a container of compressed gas, a valve, a motor, an elastomeric chamber, a flow restriction device, and a configuration of the plunger and shot chamber. In some embodiments, the damping mechanism comprises a flow restriction fluidically coupling the shot chamber and the dispensing tip. In some embodiments, the flow restriction is a constriction between the shot chamber and the dispensing tip. In some embodiments, the flow restriction is a constriction within the dispensing tip. In some embodiments, the flow restriction is a porous body. In some embodiments, the porous body comprises an open cell pore, a closed cell pore, or any combination thereof. In some embodiments, the porous body is formed of metal, ceramic, plastic, wood, or any combination thereof. In some embodiments, the flow restriction is an orifice plate within the dispensing tip, between the shot chamber and the dispensing tip, or both. In some embodiments, the flow restriction is an orifice within the dispensing tip, between the shot chamber and the dispensing tip, or both. In some embodiments, the flow restriction comprises flexible washers within the dispensing tip, between the shot chamber and the dispensing tip, or both.

In some embodiments, for any intranasal fluid delivery device described herein further comprising a secondary chamber, wherein the secondary chamber comprises a second plunger at one end configured to drive the fluid out the dispensing tip. In some embodiments, a fluidic pathway between the shot chamber and dispensing tip comprises a one-way valve configured to prevent backflow of the fluid into the shot chamber. The intranasal fluid delivery device of claim 64 or 65, wherein the second plunger is further configured to drive a secondary fluid out of the dispensing tip. In some embodiments, the secondary fluid is a gas.

In some embodiments, for any intranasal fluid delivery device described herein, further comprising an actuator operatively coupled to the plunger and configured to move the plunger, thereby enabling the plunger to drive the fluid from the shot chamber. In some embodiments, the actuator is operatively coupled to a push rod, such that when a user or the subject engages the actuator, the actuator enables the push rod to push against the plunger, thereby causing the plunger to move.

In some embodiments, for any intranasal fluid delivery device described herein, further comprising: a hollow needle coupled to the dispensing tip; and a diaphragm disposed between the shot chamber and the hollow needle, the diaphragm providing a fluidic barrier between the shot chamber and dispensing tip, wherein the hollow needle is configured to puncture the diaphragm so as to provide fluidic communication between the shot chamber and dispensing tip. In some embodiments, the hollow needle is configured to be manually pushed towards the shot chamber by a user or subject so as to puncture the diaphragm. In some embodiments, for any intranasal fluid delivery device described herein, further comprising an actuator operatively coupled to a push rod moveable toward the plunger, such that the actuator is configured to move the plunger, thereby enabling the plunger to drive the fluid from the shot chamber to the dispensing tip via the hollow needle. In some embodiments, any intranasal fluid delivery device described herein, configured such that when a user or the subject engages the actuator, the actuator enables the push rod to push against the plunger, thereby causing the plunger to move. In some embodiments, for any intranasal fluid delivery device described herein, further comprising an actuator configured such that when a user or the subject engages the actuator, the actuator enables a push rod to push against the plunger, causing the shot chamber to move toward the hollow needle, such that the hollow needle punctures the diaphragm. In some embodiments, the actuator is configured to move the plunger via the push rod, thereby enabling the plunger to drive the fluid from the shot chamber. In some embodiments, any intranasal fluid delivery device described herein, wherein a single actuation by the user of the actuator enables the shot chamber to move forward so as to puncture the diaphragm, and subsequently eject the fluid within the shot chamber via the plunger being pushed by the push rod. In some embodiments, the actuator comprises a locking mechanism, and wherein user or subject engagement of the actuator releases the locking mechanism, allowing the push rod to push against the plunger. In some embodiments, the locking mechanism comprises one or both of: one or more tabs comprising a lock material, configured such that the locking mechanism is released by the user breaking the lock material; and one or more pivotable tabs, configured such that the locking mechanism is released by the user pivoting the pivotable tabs. In some embodiments, any intranasal delivery device described herein, further comprising a spring in alignment with the push rod, wherein the locking mechanism is configured to maintain the spring under a pressure condition, wherein releasing the locking mechanism releases the spring from the pressure condition, causing the push rod to push against the plunger. In some embodiments, the spring is a variable pitch spring. In some embodiments, the locking mechanism comprises one or more tabs that break off to release the push rod, such that the device is useable only once. In some embodiments, the device is configured to maintain the fluid within the shot chamber pressurized prior to being ejected through the dispensing tip.

In some embodiments, any intranasal fluid delivery device described herein, comprising a cartridge configured for containing, or containing, a pharmaceutical fluid, wherein the cartridge comprises the shot chamber, the diaphragm, and the plunger. In some embodiments, any intranasal fluid delivery device described herein further comprising a stopping mechanism configured to limit a travel distance of the push rod. In some embodiments, any intranasal fluid delivery device described herein, further comprising a cocking mechanism configured to be activated by the user, wherein the intranasal fluid delivery device is configured such that when the user activates the cocking mechanism, pressure is applied to the spring and the spring is thereby placed under the pressure condition.

In some embodiments, any intranasal fluid delivery device described herein, further comprising a damping mechanism configured to generate a controlled velocity profile of the fluid delivered from the dispensing tip, wherein the damping mechanism comprises an actuator restriction coupled to the actuator. In some embodiments, the actuator restriction comprises a porous cavity. In some embodiments, the porous cavity comprises an open cell pore, a closed cell pore, or any combination thereof. In some embodiments, the porous cavity is formed of metal, ceramic, plastic, wood, or any combination thereof.

In some embodiments, any intranasal fluid delivery device described herein, further comprising a secondary chamber, wherein the secondary chamber comprises a second plunger at one end and a second actuator connected to a second push rod moveable toward the dispensing tip. In some embodiments, the hollow needle comprises a one-way valve configured to prevent backflow. In some embodiments, the second plunger and the second actuator are configured to drive the fluid out of the dispensing tip. In some embodiments, the second plunger and the second actuator are further configured to drive a secondary fluid out of the dispensing tip. In some embodiments, the secondary fluid is a gas. In some embodiments, any intranasal fluid delivery device described herein, further comprising a second needle for providing fluid communication between the shot chamber and the secondary chamber. In some embodiments, the second actuator is configured to control the flow rate of the fluid out of the dispensing tip.

In some embodiments, any intranasal fluid delivery device described herein, wherein the spring type, dampener type, shot chamber dimensions, dispensing tip length, or any combinations thereof is selected based on the fluid characteristics, therapeutic requirements, surface of a subject's nasal cavity, or combinations thereof.

Disclosed herein, in some embodiments, is a method for delivering a fluid to an olfactory region of a subject, the method comprising: inserting a compliant dispensing tip into the subject's nasal cavity, wherein the dispensing tip comprises a flexible nib configured to comply with or conform to a surface of the subject's nasal cavity, thereby enabling a fluid delivery orifice of the dispensing tip to be directed towards the olfactory region of the subject; and ejecting the fluid from the fluid delivery orifice of the compliant dispensing tip to deliver the fluid to the olfactory region of the subject.

In some embodiments, the dispensing tip is configured to deliver the fluid as a liquid jet or a liquid stream. In some embodiments, the liquid jet is a laminar flow or the liquid stream is a laminar flow. In some embodiments, the dispensing tip is configured to deliver the fluid as a spray, mist, or aerosol. In some embodiments, the fluid comprises a powder. In some embodiments, the dispensing tip comprises an atomizer. In some embodiments, the dispensing tip comprises a cannula. In some embodiments, n the dispensing tip is tubular. In some embodiments, the dispensing tip has an inner diameter from about 0.3 mm to about 1.5 mm. In some embodiments, the flexible nib comprises a polymer. In some embodiments, the flexible nib comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof. In some embodiments, the dispensing tip comprises a distal portion having a first rigidity and a proximal portion having a second rigidity, and wherein the first rigidity is less than the second rigidity. In some embodiments, the distal portion comprising a first rigidity comprises a portion of the dispensing tip that is about 1 mm to about 15 mm from the fluid delivery orifice. In some embodiments, the dispensing tip comprises a distal portion that is softer than a proximal portion. In some embodiments, the distal portion that is softer than a proximal portion comprises a portion of the dispensing tip that is about 1 mm to about 15 mm from the fluid delivery orifice. In some embodiments, the dispensing tip has a distal portion having a first outer diameter and a proximal portion having a second outer diameter, and wherein the first outer diameter is less than the second outer diameter. In some embodiments, the dispensing tip has a distal portion having a first outer diameter, a proximal portion having a second outer diameter, and wherein the first outer diameter is greater than the second outer diameter. In some embodiments, the dispensing tip further comprises a nose cushion to limit over-insertion of the dispensing tip within the nasal cavity, wherein the nose cushion configured to provide subject comfort. In some embodiments, the dispensing tip is configured to be inserted to an insertion depth of about 10 mm to about 90 mm within the subject's nasal cavity. In some embodiments, the fluid delivery orifice of the dispensing tip is configured to be positioned at or near the anterior entry to the olfactory region. In some embodiments, the fluid delivery orifice of the dispensing tip is configured to be positioned within about 1 mm to about 25 mm from the anterior entry to the olfactory region.

In some embodiments, for any method described herein, the dispensing tip comprises a hydrophilic coating applied to an outer surface of the dispensing tip. In some embodiments, the fluid comprises a pharmaceutical agent or a medicament. The method of claim 120, wherein the fluid comprises ketamine or insulin. In some embodiments, the pharmaceutical agent or the medicament is configured to be absorbed by the central nervous system (CNS) through the olfactory region. In some embodiments, the fluid comprises vitamins, fragrance, saline or non-pharmaceutical agents. In some embodiments, the surface of a subject's nasal cavity comprises anatomical features of a nasal cavity of the subject. In some embodiments, the anatomical features comprise nasal turbinates, a nasal valve, or combinations thereof. In some embodiments, the anatomical features comprise a septum of the subject. In some embodiments, the anatomical features comprise an anterior aspect of the nasal passage.

In some embodiments, for any method described herein, the dispensing tip has an elliptical cross section. In some embodiments, the dispensing tip has a distal portion and a proximal portion, and wherein a first center axis of the distal portion and a second center axis of the proximal portion are non-colinear. In some embodiments, the dispensing tip comprises an off-center drug dispensing channel. In some embodiments, the dispensing tip comprises a protruding element. In some embodiments, the dispensing tip comprises an inflatable balloon surrounding at least a part of a distal portion of the dispensing tip. In some embodiments, the inflatable balloon further surrounds at least a part of a proximal portion of the dispensing tip. In some embodiments, a distal portion of the dispensing tip is curved. In some embodiments, the dispensing tip has a perforation. In some embodiments, the dispensing tip has a perforation on a distal portion of the dispensing tip. In some embodiments, the perforation is on a single side of the dispensing tip. In some embodiments, a distal portion of the dispensing tip has a spiral shape. In some embodiments, exerting the pressure on fluid located within the dispensing tip enables an unraveling of the spiral shape

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Specific embodiments of the disclosed devices, delivery systems, or methods will now be described with reference to the drawings. Nothing in this detailed description is intended to imply that any particular component, feature, or step is essential to the invention.

FIG. 1 shows an example intranasal drug delivery device according to some embodiments.

FIG. 2 shows an example intranasal drug delivery device with a lid or cap according to some embodiments.

FIG. 3 shows an illustration of the olfactory region.

FIG. 4 shows examples of intranasal drug delivery devices according to some embodiments.

FIG. 5 shows an example ejection stroke and reload strokes of an intranasal drug delivery device according to some embodiments.

FIG. 6 shows an example internal view of a tip and tip mechanism of an intranasal drug delivery device according to some embodiments.

FIG. 7 shows an example intranasal drug delivery device with a removable reservoir according to some embodiments.

FIG. 8 shows an example intranasal drug delivery device according to some embodiments with the tip inserted in the nasal cavity.

FIG. 9 shows an illustration of an integrated intranasal drug-delivery platform.

FIG. 10 shows an example single use intranasal drug delivery device according to some embodiments.

FIG. 11 shows an example intranasal drug delivery device according to some embodiments.

FIG. 12 shows an example intranasal drug delivery device according to some embodiments.

FIG. 13 shows an example intranasal drug delivery device according to some embodiments.

FIG. 14 shows an example intranasal drug delivery device according to some embodiments.

FIG. 15 shows an example intranasal drug delivery device according to some embodiments.

FIG. 16 shows an external view of an example intranasal drug delivery device according to some embodiments.

FIG. 17 shows an external view of an example intranasal drug delivery device according to some embodiments.

FIG. 18 shows an example intranasal drug delivery device according to some embodiments.

FIGS. 19 a-c show an example intranasal drug delivery device according to some embodiments.

FIGS. 20 a-c show an example intranasal drug delivery device according to some embodiments.

FIG. 21 shows an example intranasal drug delivery device according to some embodiments.

FIG. 22 shows an example intranasal drug delivery device according to some embodiments.

FIG. 23 shows an example intranasal drug delivery device according to some embodiments.

FIG. 24 shows an example intranasal drug delivery device according to some embodiments.

FIG. 25 shows an example intranasal drug delivery device according to some embodiments.

FIGS. 26 a-b show an example intranasal drug delivery device according to some embodiments.

FIGS. 27 a-b show an example intranasal drug delivery device according to some embodiments.

FIG. 28 shows an example intranasal drug delivery device according to some embodiments.

FIGS. 29 a-c show an example intranasal drug delivery device according to some embodiments.

FIGS. 30 a-c show an example intranasal drug delivery device according to some embodiments.

FIG. 31 shows an example intranasal drug delivery device according to some embodiments.

FIG. 32 shows an example intranasal drug delivery device according to some embodiments.

FIGS. 33 a-c show an example intranasal drug delivery device according to some embodiments.

FIG. 34 shows an example intranasal drug delivery device with a dispensing tip having a bulbous end portion according to some embodiments.

FIG. 35 shows an example intranasal drug delivery device with a dispensing tip having an alpha loop according to some embodiments.

FIG. 36 shows images from a scan of a test subject during testing of a prototype device with a tracer fluid.

FIGS. 37A-C show an exemplary embodiment of a dispensing tip according to some embodiments used with an intranasal drug delivery device described herein.

FIGS. 38A-B show an exemplary embodiment of a dispensing tip according to some embodiments used with an intranasal drug delivery device described herein.

FIGS. 39A-B show an exemplary embodiment of a dispensing tip according to some embodiments used with an intranasal drug delivery device described herein.

FIG. 40 shows an exemplary accessory that could be used with dispensing tip described herein.

FIGS. 41A-B show an exemplary embodiment of a dispensing tip according to some embodiments used with an intranasal drug delivery device described herein.

FIGS. 42A-B show an exemplary embodiment of a dispensing tip according to some embodiments used with an intranasal drug delivery device described herein.

FIGS. 43A-D show an exemplary embodiment of a dispensing tip according to some embodiments used with an intranasal drug delivery device described herein.

FIGS. 44A-C show an exemplary embodiment of a dispensing tip according to some embodiments used with an intranasal drug delivery device described herein.

FIGS. 45A-C show an exemplary embodiment of a dispensing tip according to some embodiments used with an intranasal drug delivery device described herein.

FIGS. 46A-B show an exemplary embodiment of a dispensing tip according to some embodiments used with an intranasal drug delivery device described herein.

FIGS. 47A-C show an exemplary embodiment of a dispensing tip according to some embodiments used with an intranasal drug delivery device described herein.

FIGS. 48A-D show an exemplary method for improving patient comfort while inserting a dispensing tip used with an intranasal drug delivery device described herein into the nose.

FIGS. 49A-C show an exemplary method for improving patient comfort while inserting a dispensing tip used with an intranasal drug delivery device described herein into the nose.

FIGS. 50A-D show an exemplary method for improving patient comfort while inserting a dispensing tip used with an intranasal drug delivery device described herein into the nose.

FIGS. 51A-C show an exemplary method for improving patient comfort while inserting a dispensing tip used with an intranasal drug delivery device described herein into the nose.

FIGS. 52A-D show an exemplary method for improving patient comfort while inserting a dispensing tip used with an intranasal drug delivery device described herein into the nose.

FIGS. 53A-D show an exemplary method for improving patient comfort while inserting a dispensing tip used with an intranasal drug delivery device described herein into the nose.

FIGS. 54A-C show an exemplary method for improving patient comfort while inserting a dispensing tip used with an intranasal drug delivery device described herein into the nose.

FIGS. 55A-C show an exemplary method for improving patient comfort while inserting a dispensing tip used with an intranasal drug delivery device described herein into the nose.

FIGS. 56A-D show an exemplary method for improving patient comfort while inserting a dispensing tip used with an intranasal drug delivery device described herein into the nose.

FIG. 57 shows a damper useful to control ejection velocity from an intranasal drug delivery device described herein.

FIGS. 58A-B shows a damper useful to control ejection velocity from an intranasal drug delivery device described herein.

FIG. 59 shows a damper useful to control ejection velocity from an intranasal drug delivery device described herein.

FIG. 60 shows a damper useful to control ejection velocity from an intranasal drug delivery device described herein.

FIG. 61 shows a damper useful to control ejection velocity from an intranasal drug delivery device described herein.

FIG. 62A shows a damper useful to control ejection velocity from an intranasal drug delivery device described herein.

FIGS. 62B-D shows alternate variations to the damper of FIG. 62A.

FIG. 62E shows a relationship between velocity, force, and progressive and digressive damping profiles.

FIGS. 63A-C show an exemplary intranasal drug delivery device according to some embodiments.

FIGS. 64A-D show an exemplary intranasal drug delivery device according to some embodiments.

FIGS. 65A-C show an exemplary intranasal drug delivery device according to some embodiments.

FIGS. 66A-D show an exemplary intranasal drug delivery device according to some embodiments.

FIGS. 67A-C show an exemplary intranasal drug formulation delivery device according to some embodiments

FIGS. 68A-C show an exemplary intranasal drug formulation delivery device according to some embodiments

FIGS. 69A-D show an exemplary intranasal drug formulation delivery device dispensing tip according to some embodiments

FIG. 70 shows an exemplary intranasal drug formulation delivery device accessory according to some embodiments

FIG. 71 shows an exemplary intranasal drug formulation delivery device according to some embodiments

FIG. 72 shows an exemplary intranasal drug delivery device according to some embodiments.

FIG. 73 shows an exemplary intranasal drug delivery device according to some embodiments.

FIG. 74 shows an exemplary intranasal drug delivery device according to some embodiments.

FIGS. 75A-B show an exemplary intranasal drug delivery device according to some embodiments.

FIGS. 76A-D show an exemplary intranasal drug delivery device according to some embodiments.

FIGS. 77A-C show the intranasal drug delivery device of FIG. 76A with a cap disposed thereon.

FIGS. 78A-C show intranasal drug delivery device of FIG. 76A with a cap removed therefrom, and a trigger in an undepressed and depressed positions.

FIGS. 79A-B show intranasal drug delivery device of FIG. 76A with a ring oriented and positioned perpendicular to a longitudinal axis of the device.

### DETAILED DESCRIPTION

Embodiments of methods, systems, and apparatus are described through reference to the drawings.

Currently disposable intranasal drug delivery devices are characterized by low accuracy/uniformity of drug dosing, no design for anatomic variability and poor design for human factors—efficacy and safety. The applications where these shortcomings are most detrimental are: direct-to-brain delivery path (uptake through olfactory epithelium into CSF, action in brain), systemically acting drugs (uptake through mucosa into vasculature, systemic action), vaccines (uptake and action in mucosa), and topically acting drugs (uptake and action in mucosa).

The following provides for intranasal delivery of new and existing drugs, with the following benefits: increased dosing precision, greater efficacy, less cost, increased effectiveness, increased safety (both to patient and society), and increased convenience (in terms of health care). For any embodiment described herein, a drug comprises a fluid. In some embodiments, the fluid comprises a liquid, gel, solid, powder, or any combination thereof. In some embodiments, for any device disclosed herein, the drug delivered comprises a powder.

The following provides for opportunities in terms of design for markets where access to health care is challenged (humanitarian impact) and in terms of design for prevention of drug misuse.

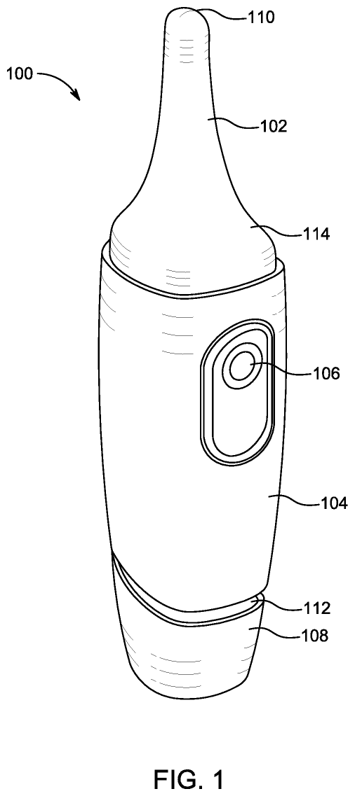

FIG. 1 shows an example intranasal drug delivery device 100 according to some embodiments.

The device 100 has a compliant or flexible, soft nib 102 (as opposed to a hard nib) to precisely locate the dosage. The soft nib 102 also provides comfort for user and may minimize blocking by the nasal wall or congestion. In some embodiments, the nib comprises a polymer. In some embodiments, the nib comprises thermoplastic polyurethane (TPU). In some embodiments, the nib comprises TPU at grade 65D, 57D, 95A, 90A, 80A, or any combination thereof. In some embodiments, the nib comprises high-density polyethylene (HDPE). In some embodiments, the nib comprises polyvinyl chloride (PVC). In some embodiments, the nib comprises a thermoplastic elastomer (TPE). In some embodiments, the nib comprises styrene-ethylene-butylene-styrene (SEBS). In some embodiments, the nib comprises low density polyethylene (LDPE). In some embodiments, the nib comprises silicone (e.g., liquid silicone rubber (LSR)). In some embodiments, the nib comprises polypropylene. In some embodiments, the nib comprises polytetrafluoroethylene (PTFE), such as for example, Teflon. In some embodiments, the nib comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof. Septal deviation can cause different health related problems. In some embodiments compliant, soft nib 102 conforms to the anterior aspect of the intranasal passage. In some embodiments the soft nib 102 is biased to follow the patient's septum. This allows the tip 110 to be placed in a location in the nasal cavity to discharge medicine targeting the olfactory region and accommodates differences in nasal cavity anatomy such as the nasal valve, nasal turbinates and the septum.

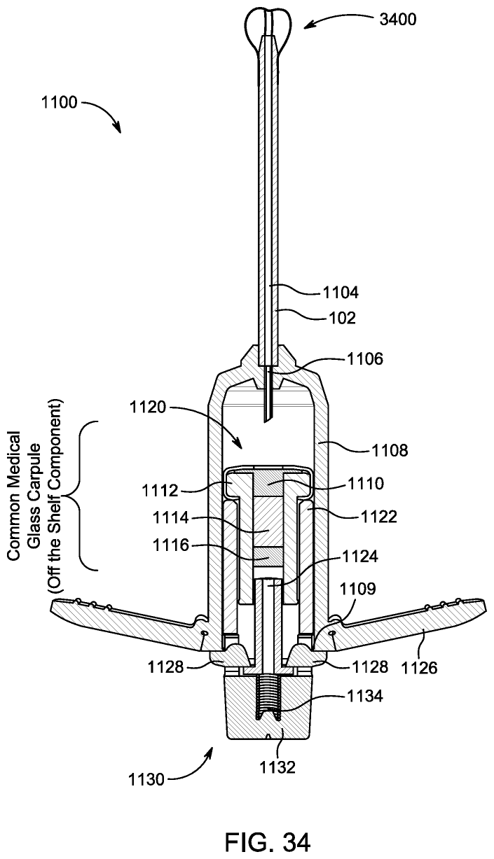

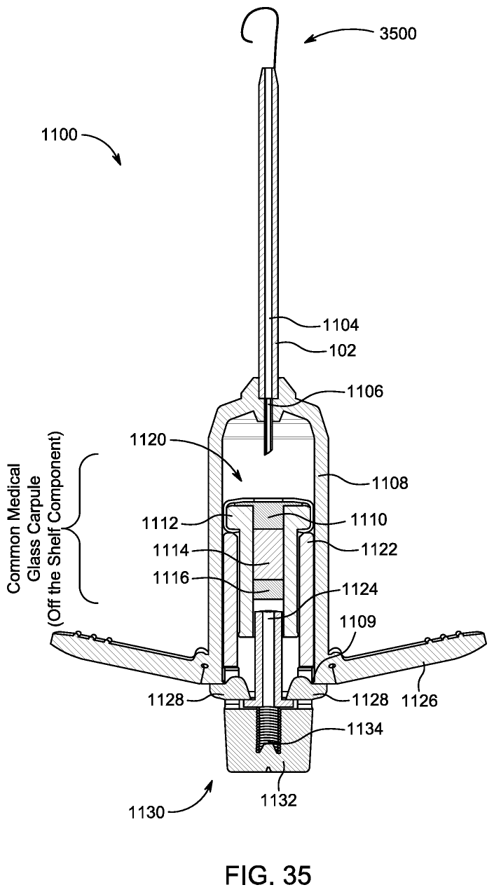

In some embodiments compliant, soft nib 102 has a kiss-cut valve near the tip 110. The valve reduces the partial discharge at the front and backend of the actuation. The tip 110 also reduces or eliminates air or contaminates from contacting the line-fill remaining in the dispensing tip between dosing. In some embodiments the orientation of the kiss cut is off set from the end of the tip 110 for directing the medicine in the direction of the olfactory region of the nasal anatomy. The nib 102 can be a multiple material over-moulded nib in some embodiments. As shown in FIG. 34, in some embodiments the nib may have a bulbous or ball shape tip 3400 to ease the insertion, improve localization, open tight passageways, and facilitate better liquid jet or stream flow to a target region in the nasal cavity. As shown in FIG. 35, in some embodiments, the compliant nib utilizes an ‘alpha loop’ 3500 to facilitate delivery past an obstruction. One of the tricks in interventional cardiology to pass a guidewire past a stricture or calcified obstruction is to force the flexible tip guide wire into the obstruction. The tip will naturally bend back on itself and the wire finds its way through the obstruction with the alpha loop leading. The larger bearing surface helps to steer the wire to the point of least resistance and it will slip through the stricture/obstruction. This embodiment may be utilized in trauma where a nose may be less than perfect, this could be the shape that would help the compliant nib find its mark.

The device 100 has an actuator 106 (e.g. button, trigger) and cocking mechanism 108 to release dosage that is reproducible to reduce human error/variation. Use of a cock-and-release mechanism in some embodiments promotes steady positioning during delivery and reduces the need for priming of the device 100, thereby reducing the possibility of operator error. In some embodiments a finger press button actuation discharges the shot chamber. This method of actuating the device 100 requires very little dexterity or fine motor skills which may be of particular importance to patients whose motor skills may be impaired e.g. patients with Parkinson's. Priming can refer to ensuring full liquid filling dosing/metering mechanism suitable for pumping of the liquid including but not limited to positive displacement pumping.

The device 100 has an internal reservoir that can be under pressure constantly in some embodiments to enable dosing independent of orientation (e.g. the user can be standing up or laying down and it will work). The reservoir may be a bag and may be collapsible by external pressure, including ambient air pressure. The pressure within the reservoir may change depending on the spring used, but it can always be under some amount of pressure.

In some embodiments the device 100 has no air-port for filling, storing or actuating the device 100. This allows for travel or transport by air, particularly unpressurized aircraft or higher elevations and may be useful for oxygen sensitive medicine and extending shelf life of certain medicines, particularly where there is no cold-chain infrastructure. In addition, this makes the device difficult to tamper with. In some embodiments, there can be an air bleed port.

In some embodiments the shape of the device 100 allows for correct dispensing tip positioning and ergonomic grip that does not engage the shoulder, wrist, or any part of the other arm not activating the device 100. The design of device 100 promotes minimal use of shoulder and arm movement.

In some embodiments the design of device 100 is made highly ergonomic in form, taking inspiration both from a wider remote controller design and a more dexterous pen design.

The ergonomics and considered human factors create a step change in the state of the art for nasal delivery devices. The design minimizes human error, allowing for a targeted, repeatable, and metered dose delivery. The design accommodates a consumable drug reservoir for short to long term use, while allowing for a low cost single patient consumable. This gives the ability for a wide variety of drugs to be filled at the point of care or by pharmaceutical filling lines. The design allows for, as an example, a compliant, soft nib 102 with an ultra-soft, matte finish, elastomeric shroud.

The compliant, soft nib 102 of the device is entered into the intranasal cavity and uses the common internal nasal geometry to guide the tip proximate to the olfactory region. The compliant soft nib 102 stops at a distance from the olfactory region and the ejected liquid containing a drug is guided to the olfactory by the native geometry of the nasal anatomy. The device mechanism supports a pocketable form being based on compact and low-cost injection-mouldable parts.

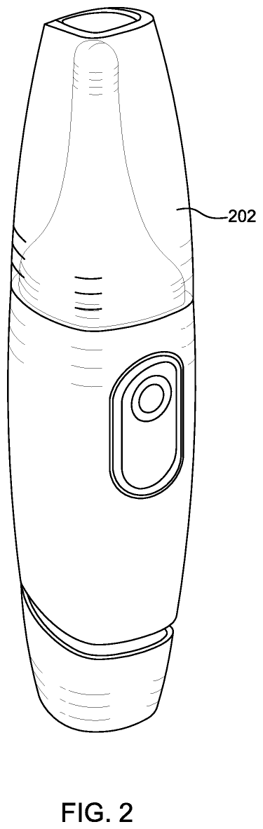

FIG. 2 shows an example intranasal drug delivery device 100 with a lid 202 or cap according to some embodiments.

In some embodiments the lid 202 may be used with the cocking mechanism 108, or instead of cocking mechanism 108, as part of reloading the intranasal drug delivery device 100. The addition of the lid 202 increases the grip size of the drug delivery device 100 and prevents misfiring of the drug delivery device 100. In some embodiments lid 202 may provide extra space for full hand grip when attached to bottom of device 100. In some embodiments lid 202 is shaped to increase the surface area without obstruction by hand when in use so that machine readable indicia (i.e. URL code) can be added to the increased surface area.

In some embodiments, the device 100 may include rechargeable energy storage to provide motive energy with separate actuation. Rechargeable energy may include electrical, chemical or pressurized fluid storage.

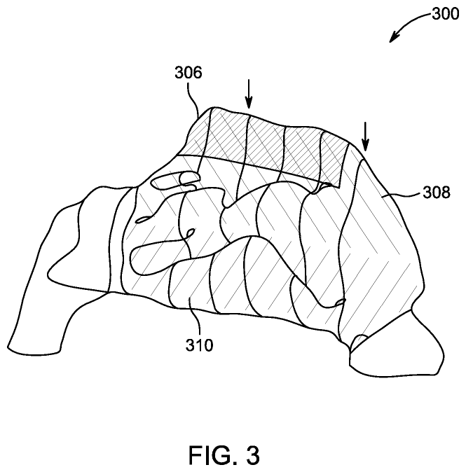

FIG. 3 shows an illustration of the nasal cavity 300 including the olfactory region 306, upper nares 308 and lower nares 310.

In topical drug delivery, drug is delivered to the entire mucosa, i.e. both the upper nares 308 and lower nares 310. In systemic drug delivery, drug is delivered through the mucosa of the upper nares 308 into the vasculature. In direct-to-brain drug delivery, drug is delivered mainly through the olfactory region 306 by diffusion into the olfactory mucosa and transport through the cribriform plate along the olfactory neuron pathways to the Central Nervous System. Drug transport from the olfactory region to the Central Nervous System may also involve the participation of trigeminal nerves.

Current drug formulations for nasal delivery use standard sprays with no specificity to the olfactory region 306, relatively small molecules are used, and formulations are mainly water-based with some alcohols. For non-active ingredients in drug formulations for nasal delivery a wide variety of functionality is used: solvents, mucoadhesive, agents, absorption enhancers, viscosity modifiers, pH buffers, antioxidants, preservatives, surfactants and more.

The majority of airflow passes through the lower nares 310. Therefore, sneezing would likely not expel liquids deposited in the olfactory region 306. Nasal congestion may affect mainly the lower nares 310 while the olfactory region 306 stays clear.

Targeted direct-to-brain drug delivery may be achieved through saturation of the olfactory region 306 with an excipient/drug combination. The drug may travel via intracellular or extracellular neuronal transport to the Central Nervous System, via the cribriform plate. This targeted delivery is intended to reduce both topical and systemic delivery, allowing for safer and more effective drug delivery.

In some embodiments the device 100 may be adapted by the addition of a lateral atomizer tip to achieve the current state of the art of topical drug delivery by saturating the entire mucosa, or systemic drug delivery by targeting the Upper Nares 308.

The olfactory plateau is generally located to the posterior aspect of the Radix line. This correlates to the Nasal Bridge length, which is measured from the soft tissue of the Nasion (Sellion) to the Subnasale.

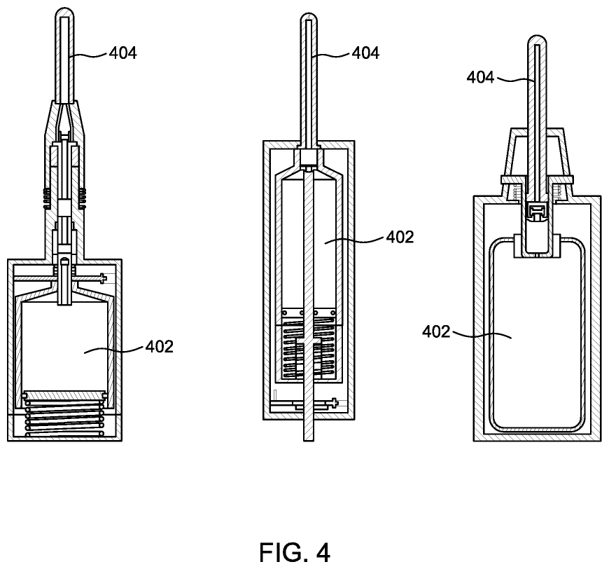

FIG. 4 shows examples of intranasal drug delivery devices according to some embodiments with reservoir 402 and compliant tip 404.

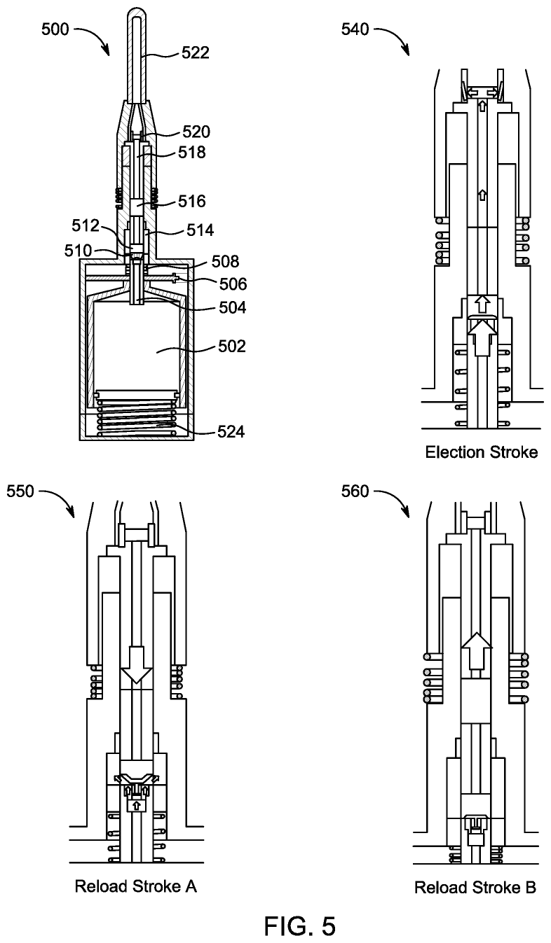

FIG. 5 shows an example release and reload mechanism 500 according to some embodiments. Release and reload mechanism 500 may be incorporated into an intranasal drug delivery devices such as, for example, device 100.

The release and reload mechanism 500 has a reservoir 502 containing a drug for delivery into the nasal cavity.

The release and reload mechanism 500 has an insertion needle 504 for insertion into the reservoir 502.

In some embodiments reservoir 502 is a bag and may be collapsible by external pressure, including ambient air pressure.

In some embodiments, reservoir 502 is removable and insertion needle 504 is inserted through a silicon stopper in the top of reservoir 502 for drawing the substance into the device 100. The silicon stopper has re-sealing properties for air sensitive medicine. The insertion needle 504 can be left in the bottle from which the medicine for the device was obtained. The filling process can eliminate the need for a separate syringe. In some embodiments, this may be referred to as a lure lock.

The release and reload mechanism 500 has actuator 506 connected to release spring 508.

The release and reload mechanism 500 has plunger 510, load valves 512 and load chambers 514.

The release and reload mechanism 500 has shot chamber 516, fluid chamber 518, release valves 520 and dispensing tip 522. The dispensing tip 522 may be in fluid communication with the nib 102 such that fluid is ejected from dispensing tip 522 and through nib 102 or as described below.

In some embodiments, release valves 520 may comprise a check valve in the dispensing tip to reduce and valve the line/dead volume. In some embodiments release valves 520 may comprise an elongated duckbill valve in tip to reduce and valve the line/dead volume.

In some embodiments, reservoir 502 is held under tension by compression spring 524. A constant and predetermined fluid pressure may be maintained by compression spring 524 pushing up from the bottom of the reservoir towards the shot chamber 516 and dispensing tip 522 and plunger 510. This constant liquid pressure charges the load chamber 514 without exposing the medicine to air or metal springs typical in most nasal pumps. In some embodiments, this may avoid the use of tubing between the reservoir 502 and shot chamber 516. This can reduce dead volume of medication or medication left in line after use. This can ensure dosing accuracy is not compromised by air entering the shot chamber 516 and no content remains in the shot chamber 516 or reservoir 502 after the last usable medicine was administered. The constant pressure enables dosing independent of user orientation.

In some embodiments the compliant, soft nib 102 is designed to discharge a liquid jet or liquid stream. In some embodiments the liquid jet or liquid stream is a laminar flow and this may include a turbulent boundary, discreet liquid slug ideally suited for maximizing dose delivery to the flat narrow section of nasal cavity leading up to the olfactory region. Delivery of laminar liquid slug assists in capillary action required for maximum medicine reaching the olfactory region. In some embodiments, the laminar stream is created by tube array or hydrodynamic focusing. In some embodiments, the liquid jet or liquid stream is delivered through the dispensing tip with a controlled velocity profile to limit shear forces on the fluid. In some embodiments, the liquid jet or liquid stream is delivered at a velocity from about 0.5 m/s to about 15 m/s. In some embodiments, the fluid is delivered at a velocity from about 1.5 m/s to about 9 m/s. In some embodiments, the fluid is delivered at a velocity from about 0.5 m/s to about 15 m/s. In some embodiments, the fluid is delivered at a velocity from about 0.5 m/s to about 1.5 m/s, about 0.5 m/s to about 3 m/s, about 0.5 m/s to about 5 m/s, about 0.5 m/s to about 9 m/s, about 0.5 m/s to about 12 m/s, about 0.5 m/s to about 15 m/s, about 1.5 m/s to about 3 m/s, about 1.5 m/s to about 5 m/s, about 1.5 m/s to about 9 m/s, about 1.5 m/s to about 12 m/s, about 1.5 m/s to about 15 m/s, about 3 m/s to about 5 m/s, about 3 m/s to about 9 m/s, about 3 m/s to about 12 m/s, about 3 m/s to about 15 m/s, about 5 m/s to about 9 m/s, about 5 m/s to about 12 m/s, about 5 m/s to about 15 m/s, about 9 m/s to about 12 m/s, about 9 m/s to about 15 m/s, or about 12 m/s to about 15 m/s, including increments therein. In some embodiments, the fluid is delivered at a velocity from about 0.5 m/s, about 1.5 m/s, about 3 m/s, about 5 m/s, about 9 m/s, about 12 m/s, or about 15 m/s. In some embodiments, the fluid is delivered at a velocity from at least about 0.5 m/s, about 1.5 m/s, about 3 m/s, about 5 m/s, about 9 m/s, or about 12 m/s. In some embodiments, the fluid is delivered at a velocity from at most about 1.5 m/s, about 3 m/s, about 5 m/s, about 9 m/s, about 12 m/s, or about 15 m/s.

In some embodiments the design of the chamber and fluid path can promote high accuracy in ejected volume.

In some embodiments device 100 is cocked by pushing down, or compressing, the bottle. This method of preparing the device for actuating requires very little dexterity or fine motor skills. This method of preparing the device for administrating medicine may be of particular importance to patients whose motor skills may be impaired e.g. patients with Parkinson's. The device can be oriented in any direction and the reloading of the shot chamber and the shot performance will not be affected i.e. the device is not gravity sensitive.

In some embodiments the compliant, soft nib 102 is extended by cocking the device. This reduces over length profile of the device for shipping, shelf space and pocketing. In the resting position the device has a less ‘menacing’ look.

In some embodiments cocking the device 100 may activate a dose counter. In some embodiments cocking may activate a separate shot counter for each dosing session.

In some embodiments cocking may activate a dose delay. In some embodiments cocking may activate a timer to remind patient when to activate between shots needed for dosing session. The delay between shots accommodates drug dosing indications including the timing of maximum drug absorption via the olfactory tight junction and the natural clearing of the mucosa cilia.

In some embodiments cocking may change the exposed color 112 between the upper bottle sleeve 104 and base 108. This, along with an extended dispensing tip (which in some embodiments does not fit in the lid 202 while cocked) gives the patient or care giver a clear visual and/or feel the device is ready for dosing or storage. In some embodiments exposed color 112 is made with glow plastic for darkness which promotes ease and convenience of nighttime use and for patients sensitive to light e.g. for administering medicine that dilates pupils.

In some embodiments the dispensing tip has an adjustable nostril stop 114. This stop gives patient feedback the dispensing tip has arrived at the optimum nostril depth. The stop also reduces sniffing/snorting during activation.

In some embodiments, the drug may be delivered by the intranasal drug delivery device 100 by delivery of a liquid jet, stream, burst or plug, rather than a spray. In some embodiments the design of the compliant, soft nib 102, the dispensing tip 522, and the valves in the reload mechanism 500 may be designed to optimize laminar ejection of drug.

Technology for liquid delivery works for a wide variety of liquid properties. This technology may be adapted to olfactory, systemic and topical delivery of drugs through an intranasal drug delivery device 100.

In some embodiments intranasal drug delivery device 100 may use particular liquid properties (such as viscosity and surface tension) to ensure prolonged residence of the delivered liquid in the target area (i.e. the olfactory region) partially enabled by capillary bridging.

In some embodiments intranasal drug delivery device 100 may include excipients in the liquid drug for delivery with particular characteristics. For example, excipients may have thixotropicity (higher viscosity at rest which improves residence time in the olfactory region 306, and lower viscosity at under shear which improves ease of metering and delivery) through additives such as cellulose. As a further example, excipients used may impact surface tension of a drug to promote wetting and capillary bridging in olfactory region. As a further example, excipients used may be pre-approved by the Federal Drug Administration for shorter development time.

In some embodiments intranasal drug delivery device 100 may include a measurement method or accessory to determine the ideal compliant, soft nib 102 size, or dispensing tip 522 type.

In some embodiments intranasal drug delivery device 100 may include a mechanical or electronic timer and/or lock mechanism to prevent overdosing. Intranasal drug delivery device 100 may incorporate use of mobile technology for identifying users and tracking use to prevent overdosing. Intranasal drug delivery device 100 may incorporate use of a cock-and-release mechanism to promote steady positioning during drug delivery. These additions assist with patient compliance.

In some embodiments intranasal drug delivery device 100 may be used in one or more of the following applications: 1) drugs directly targeting the brain via the olfactory region, 2) systemically acting drugs (e.g. better systemic bioavailability or less degradation than via the GI tract), 3) vaccines eliciting a mucosal immune response, and 4) topically-acting drugs. In some embodiments, the drug delivered is a drug formulation. In some embodiments, for any embodiment herein, the drug comprises a fluid. In some embodiments, for any embodiment described herein, the fluid comprises a liquid, gel, powder, or combinations thereof. In some embodiments, the drug comprises a powder suspended within a liquid or gaseous fluid. In some embodiments, the drug comprises a powder that is delivered by the device.

In some embodiments the intranasal drug delivery device 100 may have one or more of the following features: 1) hand held, 2) useable with a single hand, 3) designed for ambidextrous use, 4) the priming mechanism is simple and intuitive to the user, 5) there is a clear indication when the dose is primed, 6) the form promotes proper positioning in the nasal cavity, 7) designed to require a single user action to deliver a primed dose, 8) designed to prevent the user from dispensing partial doses, and 9) useable for multiple doses.

In some embodiments the intranasal drug delivery device 100 is intended to be filled by a pharmacist or other medical professional. In some embodiments the intranasal drug delivery device 100 shall contain means for preventing unintended refills of the reservoir 502.

In some embodiments the intranasal drug delivery device 100 is designed for multiple uses. In some embodiments the intranasal drug delivery device 100 uses a disposable or a refillable reservoir 502. In some embodiments the compliant, soft nib 102 is disposable.

In some embodiments intranasal drug delivery device 100 is designed with a floating gasket in a disposable or reusable reservoir 502.

In some embodiments, the drug delivery device 100 may integrate with a system involving mobile technology such as, for example, face recognition and position tracking, Gyroscopic position tracking of device and correlation with facial position, use of NFC to track number of shots.

In some embodiments, the drug delivery device 100 may enable electrically activated drug delivery such as Iontophoresis. In some embodiments, the drug delivery device 100 may involve applying an ionic charge to the drug molecule to enhance transport. In some embodiments, the drug delivery device 100 may involve an extending tip that telescopes.

In some embodiments, intranasal drug delivery device 100 is designed to use a foam as an excipient to assure residence time in target area yet allow air to pass.

In some embodiments intranasal drug delivery device 100 has barbs to lock a gasket at the end of travel to prevent misuse by refilling.

In some embodiments intranasal drug delivery device 100 has a piston that scores the chamber walls as it travels to the top of the reservoir with each actuation. This renders the device useless after a single use.

In some embodiments intranasal drug delivery device 100 is a multi-dose device with a sterile barrier to avoid contamination.

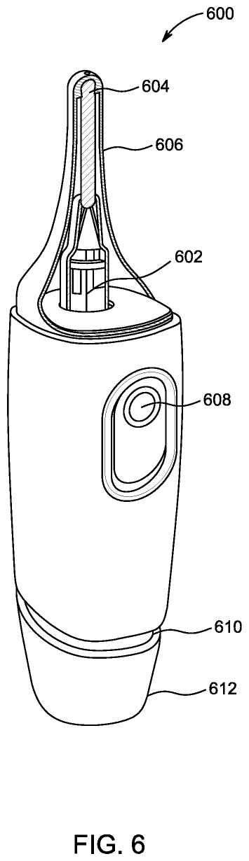

FIG. 6 shows an example intranasal drug delivery device 100 according to some embodiments including fluid chamber 602, dispensing tip 604, compliant, soft nib 606, actuator 608, exposed colour 610 and base 612.

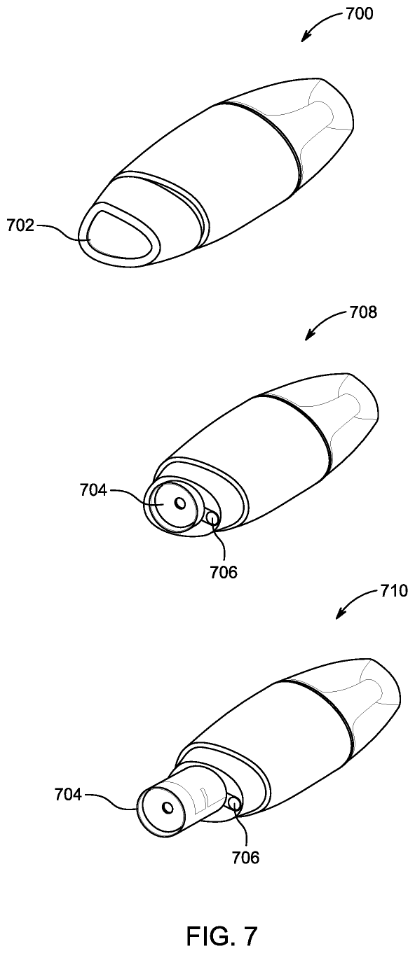

FIG. 7 shows an example intranasal drug delivery device 700 708, 710 according to some embodiments: with the base 702 connected to the intranasal drug device 700, with the base 702 removed and the removable reservoir 704 inserted into the intranasal drug delivery device 708, and with the removable reservoir 704 partially removed from intranasal drug delivery device 710. In some embodiments, a latch mechanism 706 retains the removable reservoir 704 in the device.

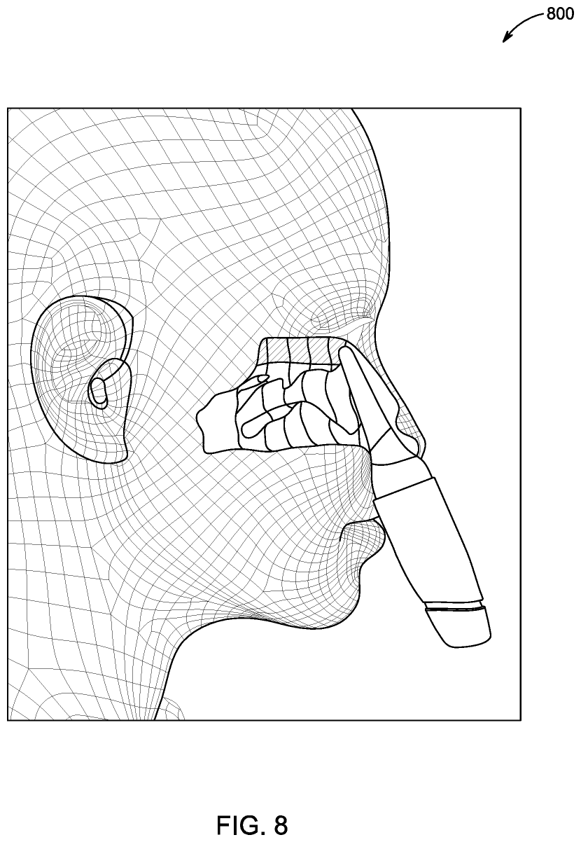

FIG. 8 shows an intranasal drug delivery device 100 inserted into the nasal cavity of a patient with the tip positioned proximate or about the olfactory region 306 or an anterior entry to the olfactory region. In some embodiments, the tip is positioned from about 0.1 mm to about 30 mm from the olfactory region or an anterior entry to the olfactory region. In some embodiments, the tip is positioned from about 0.1 mm to about 25 mm from the olfactory region or an anterior entry to the olfactory region. In some embodiments, the tip is positioned from about 0.1 mm to about 3 mm, about 0.1 mm to about 5 mm, about 0.1 mm to about 9 mm, about 0.1 mm to about 12 mm, about 0.1 mm to about 18 mm, about 0.1 mm to about 20 mm, about 0.1 mm to about 25 mm, about 3 mm to about 5 mm, about 3 mm to about 9 mm, about 3 mm to about 12 mm, about 3 mm to about 18 mm, about 3 mm to about 20 mm, about 3 mm to about 25 mm, about 5 mm to about 9 mm, about 5 mm to about 12 mm, about 5 mm to about 18 mm, about 5 mm to about 20 mm, about 5 mm to about 25 mm, about 9 mm to about 12 mm, about 9 mm to about 18 mm, about 9 mm to about 20 mm, about 9 mm to about 25 mm, about 12 mm to about 18 mm, about 12 mm to about 20 mm, about 12 mm to about 25 mm, about 18 mm to about 20 mm, about 18 mm to about 25 mm, or about 20 mm to about 25 mm, including increments therein. In some embodiments, the tip is positioned from about 0.1 mm, about 3 mm, about 5 mm, about 9 mm, about 12 mm, about 18 mm, about 20 mm, or about 25 mm. In some embodiments, the tip is positioned from at least about 0.1 mm, about 3 mm, about 5 mm, about 9 mm, about 12 mm, about 18 mm, or about 20 mm. In some embodiments, the tip is positioned from at most about 3 mm, about 5 mm, about 9 mm, about 12 mm, about 18 mm, about 20 mm, or about 25 mm from the olfactory region or an anterior entry to the olfactory region. In some embodiments, the tip is touching the olfactory region. In some embodiments a speculum may be used as an accessory to open the nostril. In some embodiments the device 100 may be include an accessory part to guide the tip.

The compliant, soft nib 102 of the device is entered into the intranasal cavity and uses the common internal nasal geometry to self-guide the compliant, soft nib 102 towards the olfactory region. The compliant, soft nib 102 is held from lateral deviation via the flanking medial septum, and the lateral nasal wall.

In some embodiments when the device 100 is activated, an internal metering chamber ejects a repeatable and metered dose into the superior/posterior aspect of the olfactory region. In some embodiments, a liquid jet or liquid stream is produced, as opposed to conventional spray, mist or aerosol, to ensure that the ejected dose gets delivered to the target area, rather than spreading in the entire intranasal space. In some embodiments, a laminar flow is produced. In some embodiments, due to the Coanda effect, the ejected excipient adheres to the medial, lateral and superior aspect of the olfactory corridor while still motive.

In some embodiments, when the motive energy of the ejected liquid has dissipated, the excipient coats part of or all of the olfactory region surface. In some embodiments, when the motive energy of the ejected liquid has dissipated, opposing wall capillary motion allows the excipient to fill a part of the entire olfactory region. This is due to the combination of excipient surface tension (which is caused by cohesion within the excipient) and mucoadhesive properties between the excipient and olfactory mucosa wall.

To achieve residence time, and as a result of capillary action, the excipient will be held in the olfactory corridor due to a capillary bridge effect caused by the opposing walls of the medial, lateral and superior aspect of the olfactory corridor, thus preventing the excipient from draining to the inferior aspect of the nasal vault. An adequately high viscosity or thixotropic property of the excipient helps prolonging residence time.

In one embodiment the proposed method for targeted drug delivery using the device 100 is as follows: 1) The compliant tip is placed to the anterior aspect of the olfactory corridor; 2) The excipient is ejected out of the tip in a liquid jet or liquid stream and towards the posterior aspect of the olfactory region; 3) When the motive energy of the ejected liquid has dissipated, the excipient coats all or a portion of the olfactory region surface. This is due to the combination of excipient surface tension (which is caused by cohesion within the excipient) and mucoadhesive properties between the excipient and olfactory mucosa wall. In one embodiment the proposed method for targeted drug delivery using the device 100 is as follows: 1) The compliant tip is placed to the anterior aspect of the olfactory corridor; 2) The excipient is ejected out of the tip in a liquid jet or liquid stream and towards the posterior aspect of the olfactory corridor; 3) When the motive energy of the ejected liquid has dissipated, opposing wall capillary motion allows the excipient to fill all or a portion of the entire olfactory region. This is due to the combination of excipient surface tension (which is caused by cohesion within the excipient) and mucoadhesive properties between the excipient and olfactory mucosa wall; 4) To achieve residence time, and as a result of capillary action, the excipient will be held in the olfactory region due to a capillary bridge effect caused by the opposing walls of the medial, lateral and superior aspect of the olfactory region, thus preventing the excipient from draining to the inferior aspect of the nasal vault. An adequately high viscosity or thixotropic property of the excipient helps prolonging residence time. In some embodiments, the liquid jet is a “reasonably” laminar jet.

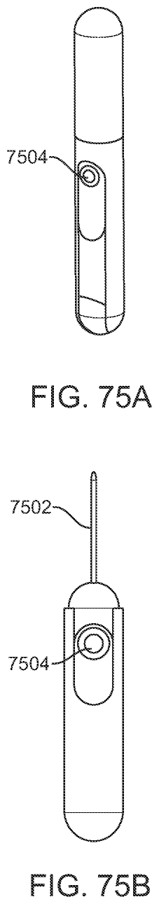

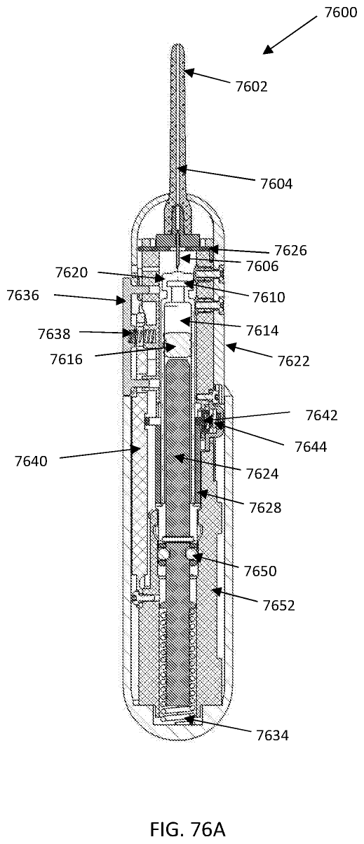

FIGS. 75A-B illustrate another exemplary embodiment of an intranasal drug delivery device 7500 according to some embodiments. The device 7500 comprise a dispensing tip 7502 as described herein. In some embodiments, the device 7500 further comprises a trigger 7504 for delivery of a fluid to within a nasal cavity of a subject.

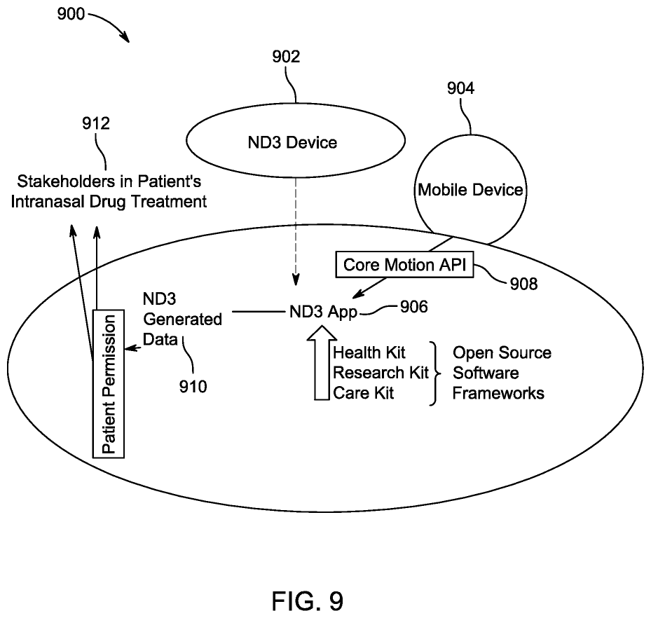

FIG. 9 illustrates an integrated intranasal drug-delivery platform 900 including an intranasal drug delivery device 902, a mobile device 904, an intranasal device software application 906, a core application program interface 908, and device generated data 910 that may be shared with shareholders 912.

The device 902 can connect to a software application 906 installed on a mobile device 904 for data logging to flag or track misuse and compliance. For example, the intranasal device software application 906 can capture images up the nasal cavity to flag misuse, implement user biometric authentication for compliance, capture timing data of dosage for compliance, provide alerts or reminders to user and so on.

In some embodiments a software application will be available in association with the device 100 to create an integrated hardware and software intranasal drug-delivery platform 900. This includes a database for the storage of data generated from device 100 that serves as a basis for extension to a permission-based personal data ecosystem platform.

In some embodiments the software application may be extended to become a platform for more broad data aggregation and permission-based sharing. A patient's personal data could be collected and exchanged with permission to/from all parties who have a role and accountability for administering (dispensed and applied) intranasal treatments. The data exchange portal would provide patient insight aimed at aligning and continuously influencing positive behavior for optimum health care delivery. The extension will facilitate sharing of different types of smartphone-based personal data to different stakeholders such as other patients, guardians, doctors, clinics, clinical trial researches, health care providers, patient medical insurers, doctor insurers, health care insurers, drug developers, pharmacies, patient peer support groups, disease/disorder researchers, disease/disorder NGO's, government regulators, law enforcement/first responders. Privacy and control of personal data are important. A user may wish to share data in certain circumstances, based on incentives or goodwill.

In some embodiments components of an integrated intranasal drug-delivery platform 900 may comprise an intranasal drug delivery device 902 that is inextricably linked with a specified medicine and an individual patient through device and patient verification; intranasal drug delivery device 902 that provides machine readable signals (fiducial markers) at time of scrip writing, scrip filling, patient dosing, patient possession, and device redemption (i.e. patient life cycle events); on-going data harvesting, transit, storage and retrieval capability; aggregation and anonymization of personal data into mineable and usable data sets e.g. reporting, analytics, gamification, incentivizing, etc.; personal data for optimizing patient's immediate and ongoing healthcare and a permission-based sharing system.

Categories of data that an integrated intranasal drug-delivery platform 900 may utilize include a patient profile; stakeholder profiles to manage data that has been shared with them; non-medical passive personal data (recovery of which may be ongoing); medical/biometric personal data (recovery of which may be ongoing); event driven personal data at time of scrip writing, scrip filling, patient dosing, patient possession, and device redemption (i.e. patient lifecycle); and event driven prompting to influence immediate behavior.

For an example of an integrated intranasal drug-delivery platform 900 for a user that has been prescribed a drug that is dispensed with intranasal drug delivery device 902, 1) the user receives an alert on his/her mobile device 904 signaling that it's time to take a scheduled dose of drug, 2) the user unlocks the mobile device 904 using native identity authentication (passcode, fingerprint or facial recognition) and the intranasal device software application 906 opens on the mobile device, 3) the user touches the mobile device 904 to the intranasal drug delivery device 902 or initiates another form of recognition, 4) the user uses the mobile device 902 for facial recognition validation, 5) the intranasal device software application 906 prompts the user for measuring pre-actuation metrics/biometrics (relevant metrics may be determined by clinician, for example, cognition survey, HR measurement, short video capture to determine emotional state/impairment etc.), 6) the user completes any inputs needed to complete pre-actuation tests, 7) the intranasal device software application 906 determines that the intranasal drug delivery device 902 has been actuated (the action may be timestamped and recorded, methods for confirming actuation include Bluetooth connectivity, visual image, sound, colour change, artificial intelligence that recognizes actuation), 8) the intranasal device software application 906 prompts the user for measurements of post-actuation biometrics (relevant metrics may be determined by clinicians); 9) the user is taken back to dashboard as part of an interface controlled by software application 906 where he/she can track different metrics and manage permissions (who can see what data).

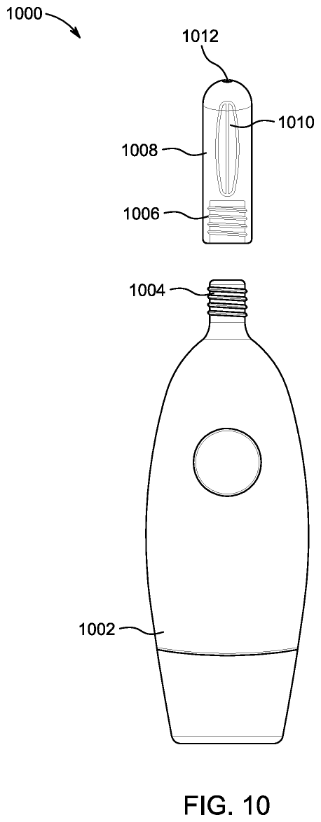

FIG. 10 shows an example single use intranasal drug delivery device 1000, pump 1002 incorporating a reservoir, a pump locking mechanism 1004, and compliant, soft nib 1008, with a nib locking mechanism 1006, shot chamber 1010 and spray tip 1012. In some embodiments the pump 1002 would be a spring actuated piston and the pump locking mechanism 1004 would lock with the nib locking mechanism 1006.

In some embodiments the device can include an olfactory marker that will be included with the excipient/drug that will provide biofeedback to the user. This may take the form of olfactory active marker that can signal to the user that the drug/excipient has been delivered to the olfactory region. This may include, but not be limited to markers which provide feedback of missed, un-deployed, deployed or over deployed drug/excipient. The marker can be included in the drug/excipient formulation or in some embodiments be added during the ejection process. In some embodiments, the marker may be included without the active drug agent to provide feedback to the user that an application and dosage (without the drug agent) was successful soliciting a psychological response.

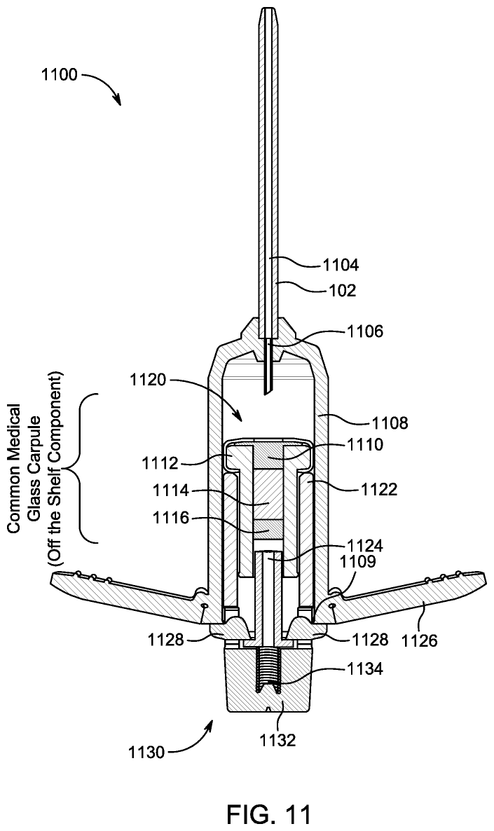

FIG. 11 shows an example intranasal drug delivery device 1100 according to some embodiments. The device 1100 comprises an outer chassis 1108 with a dispensing opening at a first end and an actuating opening at a second end. A dispensing tip is coupled to the dispensing opening, and an actuator 1130 is coupled to the actuating opening. As described below, fluid can be delivered to a nasal volume through the dispensing tip by pressing on the actuator 1130. In some embodiments, for any intranasal drug delivery device described herein, the fluid comprises a liquid, a gel, a powder, or any combinations thereof. In some embodiments, the fluid is delivered from the dispensing tip through a fluid delivery orifice disposed at a distal end of the dispensing tip. In some embodiments, the drug comprises a powder that is delivered by the device.

In some embodiments, the device 1100 is configured to receive a cartridge, which may be pre-filled with a fluid for delivery into an intranasal cavity of a subject. In some embodiments, for any intranasal delivery device described herein, a cartridge comprises a carpule. In some embodiments, the cartridge 1120 (which comprises a diaphragm 1110, tube 1112, shot chamber 1114, and plunger 1116 as described below) pre-filled with a fluid, such as for example a pharmaceutical fluid. In the FIG. 11 example, the device 1100 comprises an enclosure 1122 slidably received within the outer chassis 1108 and shaped to accept a cartridge 1120.

The cartridge 1120 comprises a tube 1112 with an interior shot chamber 1114 that contains a fluid. In some embodiments, shot chamber 1114 may carry medication, such as ketamine of other pharmaceuticals, for delivery to a patient's nasal cavity or olfactory region. In some embodiments, for any device disclosed herein, the shot chamber is removable from the cartridge. In some embodiments, the shot chamber may be removed and replaced with another shot chamber. In some embodiments, the shot chamber is refillable. In some embodiments, the shot chamber comprises a refillable cartridge. The shot chamber 1114 has a plunger 1116 on one end, and a diaphragm 1110 on the opposite end from the plunger 1116. The device 1100 is configured such that when a user engages the actuator 1130, the fluid in the shot chamber 1114 is delivered through the dispensing tip with predetermined flow characteristics. In some embodiments, the dispensing tip comprises a flexible cannula or nib 102 configured to deliver a liquid jet, stream, burst or plug. In some embodiments, the liquid jet comprises laminar flow. In some embodiments, the dispensing tip comprises a flexible cannula or nib 102 configured to deliver a laminar liquid slug, as described above. In some embodiments, the fluid is delivered through the dispensing tip with a controlled velocity profile to limit shear forces on the fluid. In some embodiments, the fluid is delivered at a velocity from about 0.5 m/s to about 15 m/s. In some embodiments, the fluid is delivered at a velocity from about 1.5 m/s to about 9 m/s. In some embodiments, the fluid is delivered at a velocity from about 0.5 m/s to about 15 m/s. In some embodiments, the fluid is delivered at a velocity from about 0.5 m/s to about 1.5 m/s, about 0.5 m/s to about 3 m/s, about 0.5 m/s to about 5 m/s, about 0.5 m/s to about 9 m/s, about 0.5 m/s to about 12 m/s, about 0.5 m/s to about 15 m/s, about 1.5 m/s to about 3 m/s, about 1.5 m/s to about 5 m/s, about 1.5 m/s to about 9 m/s, about 1.5 m/s to about 12 m/s, about 1.5 m/s to about 15 m/s, about 3 m/s to about 5 m/s, about 3 m/s to about 9 m/s, about 3 m/s to about 12 m/s, about 3 m/s to about 15 m/s, about 5 m/s to about 9 m/s, about 5 m/s to about 12 m/s, about 5 m/s to about 15 m/s, about 9 m/s to about 12 m/s, about 9 m/s to about 15 m/s, or about 12 m/s to about 15 m/s, including increments therein. In some embodiments, the fluid is delivered at a velocity from about 0.5 m/s, about 1.5 m/s, about 3 m/s, about 5 m/s, about 9 m/s, about 12 m/s, or about 15 m/s. In some embodiments, the fluid is delivered at a velocity from at least about 0.5 m/s, about 1.5 m/s, about 3 m/s, about 5 m/s, about 9 m/s, or about 12 m/s. In some embodiments, the fluid is delivered at a velocity from at most about 1.5 m/s, about 3 m/s, about 5 m/s, about 9 m/s, about 12 m/s, or about 15 m/s.

As described herein, in some embodiments and for any intranasal delivery device described herein, the dispensing tip is configured to be inserted into a subject's nasal cavity. In some embodiments, the dispensing tip comprises a fluid discharge orifice configured to discharge fluid from the dispensing tip to the olfactory region. As described herein, in some embodiments, the dispensing tip comprises a flexible nib or cannula configured to comply with or conform to a surface of a subject's intranasal passage, thereby enabling the fluid delivery orifice of the dispensing tip to be positioned within the intranasal cavity of a subject. In some embodiments, the dispensing tip is configured to be positioned in or near an olfactory region of the subject. In some embodiments, the dispensing tip is configured to be inserted within a subject's nasal cavity to an insertion depth of at least about 10 mm to about 85 mm. In some embodiments, the dispensing tip is configured to be inserted within a subject's intranasal cavity to an insertion depth of about 5 mm to about 100 mm. In some embodiments, the dispensing tip is configured to be inserted within a subject's intranasal cavity to an insertion depth of about 5 mm to about 25 mm, about 5 mm to about 50 mm, about 5 mm to about 70 mm, about 5 mm to about 85 mm, about 5 mm to about 100 mm, about 25 mm to about 50 mm, about 25 mm to about 70 mm, about 25 mm to about 85 mm, about 25 mm to about 100 mm, about 50 mm to about 70 mm, about 50 mm to about 85 mm, about 50 mm to about 100 mm, about 70 mm to about 85 mm, about 70 mm to about 100 mm, or about 85 mm to about 100 mm, including increments therein. In some embodiments, the dispensing tip is configured to be inserted within a subject's intranasal cavity to an insertion depth of about 5 mm, about 25 mm, about 50 mm, about 70 mm, about 85 mm, or about 100 mm. In some embodiments, the dispensing tip is configured to be inserted within a subject's intranasal cavity to an insertion depth of at least about 5 mm, about 25 mm, about 50 mm, about 70 mm, or about 85 mm. In some embodiments, the dispensing tip is configured to be inserted within a subject's intranasal cavity to an insertion depth of at most about 25 mm, about 50 mm, about 70 mm, about 85 mm, or about 100 mm.

In some embodiments, for any embodiment herein, the dispensing tip comprises a distal portion that is softer than a proximal portion. In some embodiments, the distal portion that is softer than a proximal portion comprises a portion of the distal tip between about 1 mm to about 15 mm from a distal end of the dispensing tip. In some embodiments, for any embodiment herein, the dispensing tip comprises a distal portion having a first rigidity and a proximal portion having a second rigidity, and wherein the first rigidity is less than the second rigidity. In some embodiments, the distal portion comprising a first rigidity comprises a portion of the dispensing tip that is about 1 mm to about 15 mm from a distal end of the dispensing tip. In some embodiments, the dispensing tip further comprises a nose cushion to limit over-insertion of the dispensing tip within the intranasal passage. In some embodiments, the nose cushion is configured to provide a user comfort when the dispensing is inserted into a user's intranasal cavity. In some embodiments, the nose cushion is removably attached to the dispensing tip. In some embodiments, the dispensing tip has a proximal portion having an outer diameter that tapers towards the distal portion to provide comfort for the user or to limit insertion distance.

In some embodiments, the dispensing tip comprises a polymer. In some embodiments, the dispensing tip comprises thermoplastic polyurethane (TPU). In some embodiments, the dispensing tip comprises TPU at grade 65D, 57D, 95A, 90A, 80A, or any combination thereof. In some embodiments, the dispensing tip comprises high-density polyethylene (HDPE). In some embodiments, the dispensing tip comprises polyvinyl chloride (PVC). In some embodiments, the dispensing tip comprises a thermoplastic elastomer (TPE). In some embodiments, the dispensing tip comprises styrene-ethylene-butylene-styrene (SEBS). In some embodiments, the dispensing tip comprises low density polyethylene (LDPE). In some embodiments, the dispensing tip comprises silicone (e.g., liquid silicone rubber (LSR)). In some embodiments, the dispensing tip comprises polypropylene. In some embodiments, the dispensing tip comprises polytetrafluoroethylene (PTFE), such as for example, Teflon. In some embodiments, the dispensing tip comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof.

In some embodiments and for any intranasal delivery device described herein, the dispensing tip comprises an inner diameter of at most about 1.0 mm. In some embodiments, the dispensing tip comprises an inner diameter of at most about 0.7 mm. In some embodiments, the dispensing tip comprises an inner diameter from about 0.5 mm to about 1.0 mm. In some embodiments, the dispensing tip comprises an inner diameter from about 0.3 mm to about 1.5 mm. In some embodiments, the dispensing tip comprises an inner diameter of at least about 0.3 mm.

In some embodiments, plunger 1116 may be engaged by a push rod 1124. In the FIG. 11 example, the push rod 1124 is located at the bottom of the enclosure 1112, and a spring 1134 is compressed between the push rod 1124 and a push button 1132. In some embodiments, the spring is a variable pitch spring. A locking mechanism 1128 holds the push rod 1124 and prevents it from engaging with plunger 1116 until the push button 1132 is pressed. In the illustrated example, the locking mechanism 1128 comprise a pair of pivotable tabs with inner ends engaging the push rod and outer ends extending past the outer edges of the enclosure 1122 such that when the enclosure 1122 is pushed into the chassis 1108 by pressing on the push button 1132 the tabs pivot to release the push rod 1124. In other embodiments, the locking mechanism may comprise one or more tabs of a lock material which is breakable by pressing on the push button 1132.

The diaphragm 1110 is puncturable by the needle 1106. Needle 1106 connects to channel 1104 in flexible nib 102, which may be inserted into the nasal cavity for fluid delivery as described above. When engaged, the fluid in shot chamber 1114 is forced through needle 1106 and channel 1104 into the nasal cavity. Arms 1126 may assist the user in gripping device 1100 and engaging push button 1132.

In some embodiments, to assemble device 1100, cartridge 1120 may be inserted into the cartridge enclosure 1122. The cartridge enclosure 1122 may then be inserted into outer chassis 1108. In the illustrated example, the chassis 1108 comprises a resilient lip 1109 and the actuator opening deforms slightly to receive the cartridge enclosure 1122 and cartridge 1120, then holds them within the chassis 1108. In other embodiments, seals may be added to assist in detection of tampering.

Use of a cartridge may be advantageous in certain situations because it is a commonly manufactured vessel for medication and may be made of a material that is non-reactive with medication, such as glass.

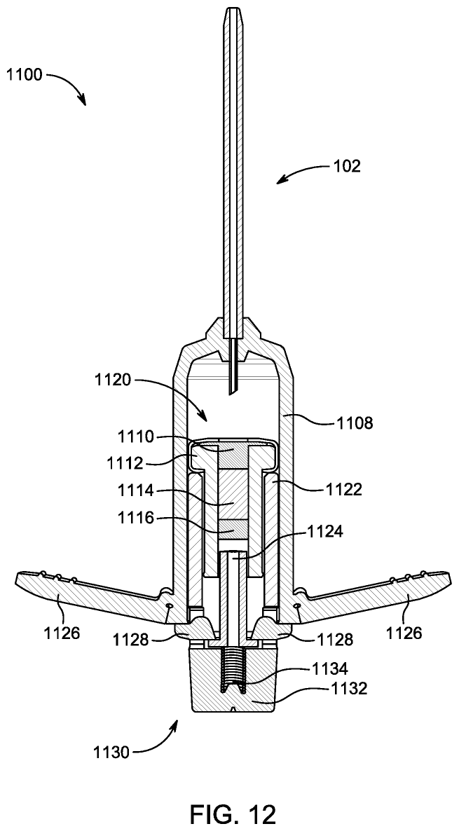

FIG. 12 shows an example intranasal drug delivery device 1100 according to some embodiments, wherein cartridge 1120 is inserted in cartridge enclosure 1122 and the cartridge enclosure 1122 is inserted in outer chassis 1108, but the actuator 1130 has not been engaged by the user and locking mechanism 1128 holds push rod 1124 such that plunger 1116 is not engaged and fluid in shot chamber 1114 is not under pressure. Arms 1126 may be folded outward or inward against outer chassis 1108. The device 1100 may be stored without the fluid in shot chamber 1114 being under pressure. Flexible nib 102 may be placed in the nasal cavity of the patient prior to the actuator 1130 being engaged by the user.

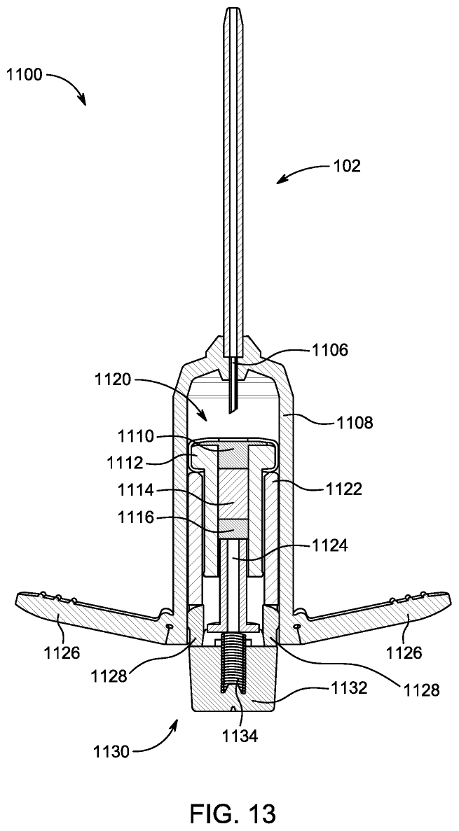

FIG. 13 shows an example intranasal drug delivery device 1100 according to some embodiments, wherein the user has engaged the push button 1132, for example, by pushing it with their thumb. The user may hold the device 1100 in their hand using arms 1126 in a folded out orientation. When user pushes the push button 1132, the locking mechanism 1128 releases push rod 1124. In some embodiments, the locking mechanism may comprise one or more tabs that break off to release push rod 1124, making the device 1100 useable only once. In other embodiments, the locking mechanism may comprise one or more tabs that fold or cantilever out of the way to release push rod 1124. When the locking mechanism 1128 is engaged it prevents the push rod 1124 from exerting pressure on the plunger 1116.

When the push rod 1124 presses against the plunger 1116 it puts the fluid in shot chamber 1114 under pressure, and will move the cartridge 1120 toward the needle. In some embodiments, a spring 1134 may be included to such that the push rod 1124 exerts even pressure on plunger 1116, and once the locking mechanism 1128 is released the spring 1134 will cause cartridge 1120 to move further into outer chassis 1108 toward needle 1106 until needle 1106 punctures diaphragm 1110. In some embodiments a user continues to push on the push button 1132 to move the cartridge 1120 into outer chassis 1108 until the needle 1106 punctures diaphragm 1110.

In some embodiments, actuator 1130 may be a push button located at the bottom of device 1100, in other embodiments, actuator 1132 may be located on the side of outer chassis 1108.

In some embodiments, device 1100 may be designed for one-time use, with a locking mechanism 1128 comprising tabs that break off, or other sacrificial clips or structures such that cartridge enclosure 1122 may not be removed from outer chassis 1118 to replace the spent cartridge 1120 with a new cartridge 1120 without the device 1100 being damaged.

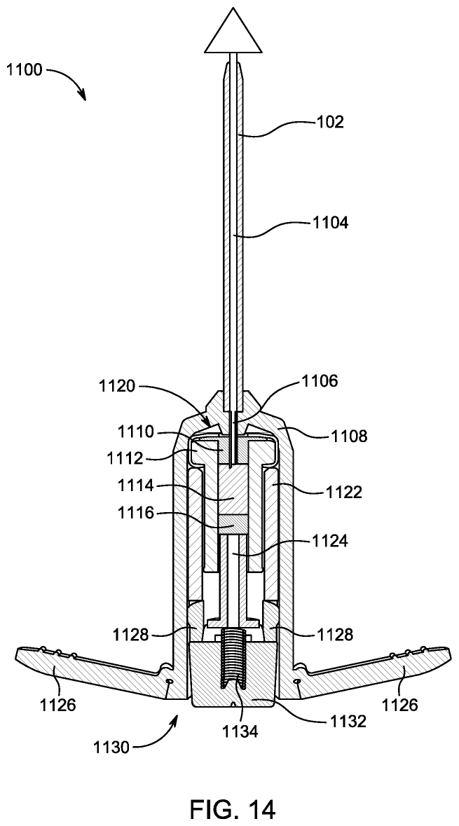

FIG. 14 shows an example intranasal drug delivery device 1100 according to some embodiments, wherein the user has pushed the actuator 1130 such that it causes the needle 1106 to puncture diaphragm 1110 so that the tip of needle 1106 is in contact with the fluid in shot chamber 1114. The fluid in shot chamber 1114 is under pressure from the plunger 1106 and may enter needle 1106 and flow through channel 1104 in nib 102. Fluid may flow through channel 1104 to be deposited in the nasal cavity or olfactory region of a patient.

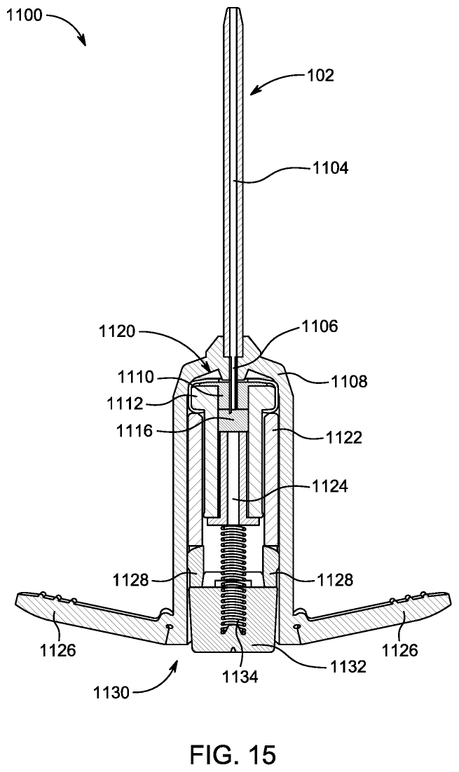

FIG. 15 shows an example intranasal drug delivery device 1100 according to some embodiments, wherein the user has pushed the actuator 1130 such that push rod 1124 has pushed plunger 1116 to reach diaphragm 1110, ending the ejection of fluid. In some embodiments, upon pushing the actuator 1130, the push rod will first push the cartridge such that the needle penetrates the diaphragm, followed by the plunger being pushed by the push rod to discharge the contents of the shot chamber. In some embodiments, upon pushing the actuator 1130, the push rod will push the cartridge such that the needle penetrates the diaphragm, and the plunger being pushed by the push rod will discharge the contents of the shot chamber, wherein the device is actuate through a single actuation (or engagement) by the user.

In some embodiments, an intranasal drug delivery device as depicted in FIG. 11 is provided, wherein the cartridge does not comprise a diaphragm, such that actuation of the device will engage the push rod with the plunger, thereby pushing the fluid in the shot chamber through the dispensing tip and into the nasal cavity or olfactory region of a patient.

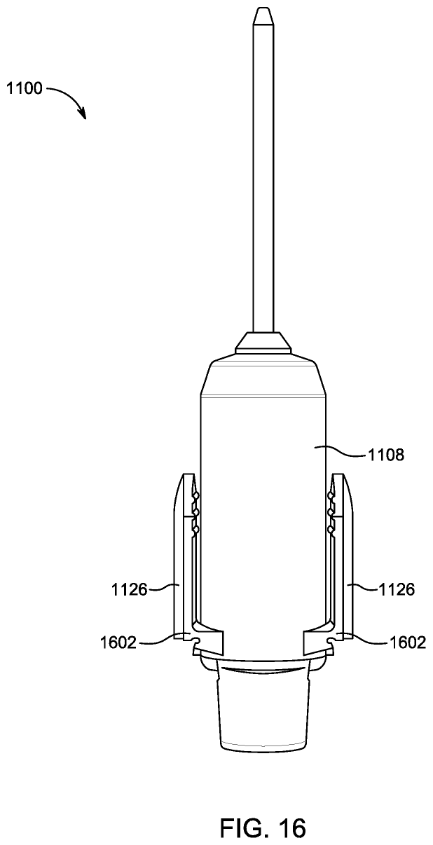

FIG. 16 shows an external view of an example intranasal drug delivery device 1100 according to some embodiments, wherein arms 1126 are hinged with hinge 1602 and may be folded against outer chassis 1108 for storage, packing and transport. Hinge 1602 may be a living hinge comprised of thin material, for example.

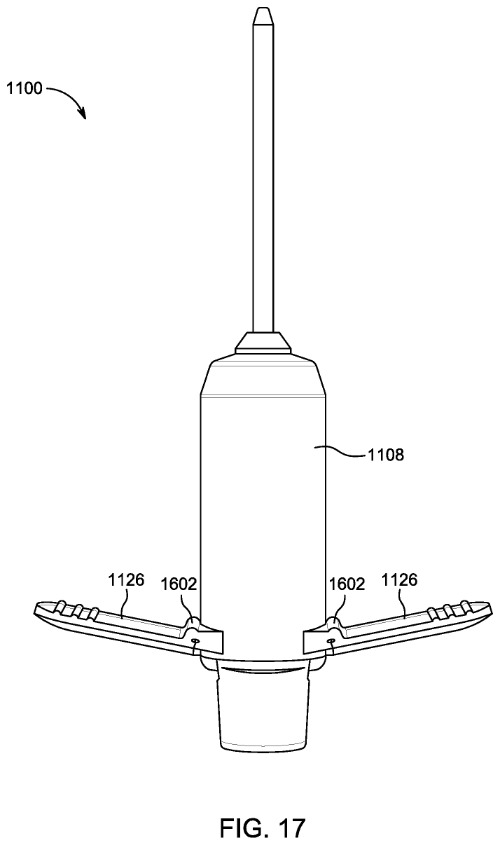

FIG. 17 shows an external view of an example intranasal drug delivery device 1100 according to some embodiments, wherein arms 1126 are folded outward from the outer chassis 1108, providing a grip for the user when using the device 1100. In the folded out position arms 1126 may provide a grip for a user wearing gloves or a user with dexterity challenges.

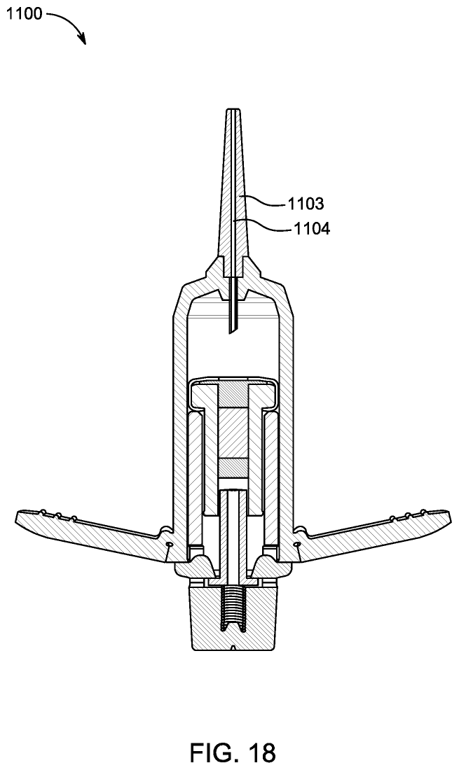

FIG. 18 shows an example intranasal drug delivery device 1100 according to some embodiments, wherein the dispensing tip comprises an atomizer 1103 designed to deliver a spray of fluid into the nasal cavity rather than a laminar liquid slug.

In some embodiments, components and the configuration for any intranasal drug delivery device disclosed herein can be specified based on delivery fluid characteristics, therapeutic requirements, physiology of a patient's nasal anatomy, or combinations thereof, so as to improve drug delivery accuracy. In some embodiments, fluid characteristics include volume, viscosity, density, weight, or combinations thereof. In some embodiments, components and configuration that can be specified include spring type and characteristics, dimensions of the shot chamber, dispensing tip length, dispensing tip flexibility, damper type, or combinations thereof.

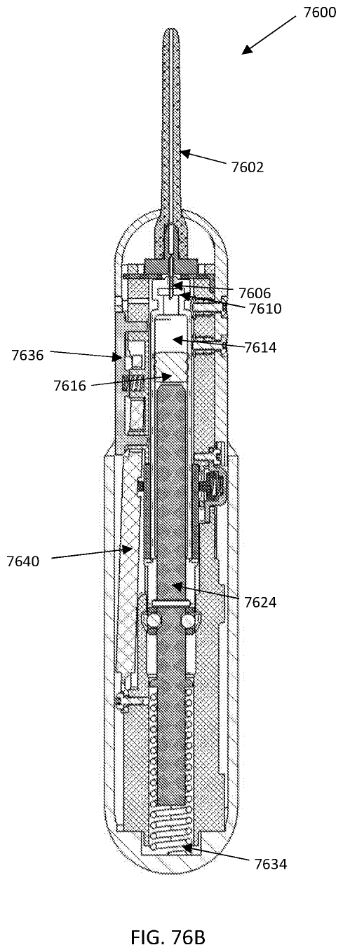

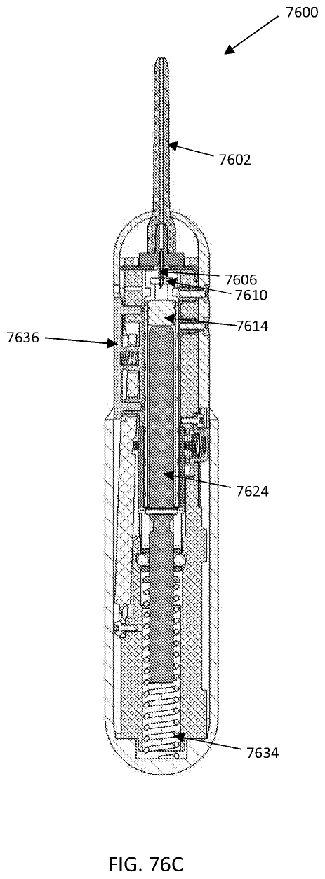

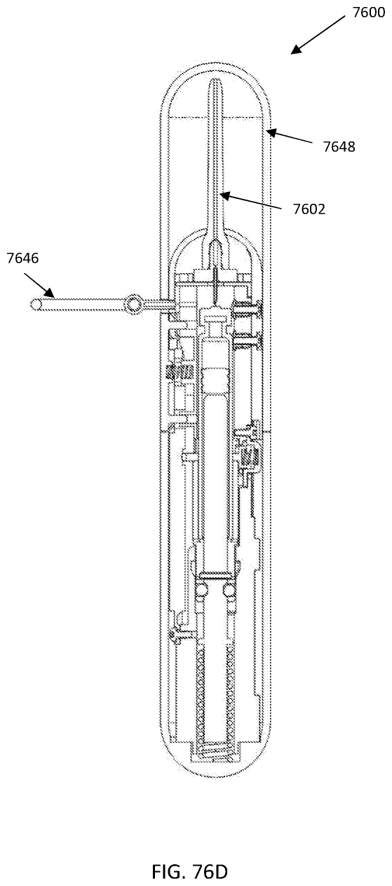

FIGS. 76A to 79B illustrate an exemplary embodiment of an intranasal drug delivery device 7600 according to some embodiments. FIG. 76A shows the device 7600 comprising a dispensing tip 7602. In some embodiments, the dispensing tip is flexible, and configured to comply with an intranasal cavity of a subject, as described herein. In some embodiments, the dispensing tip comprises a nib. The dispensing tip may comprise a channel 7604. In some embodiments, the device 7600 is configured to receive a cartridge, which may be pre-filled with a fluid for delivery into an intranasal cavity of a subject. The device may further comprise a cartridge holder 7628. As described herein, in some embodiments, a cartridge comprises a cartridge 7620. In some embodiments, the cartridge 7620 comprises a diaphragm 7610, shot chamber 7614, and plunger 7616. In some embodiments, the shot chamber 7614 is pre-filled with a fluid, such as for example a pharmaceutical fluid. In some embodiments, the device comprises a needle 7606 configured to penetrate the diaphragm 7610.

The device 7600 may further be configured with a trigger 7636 to depress a trigger paddle 7640. In some embodiments, the trigger 7636 is configured with a trigger spring 7638 that is configured to return the trigger 7636 to its original position. In some embodiments, the trigger spring 7638 is a compliant member built directly into the trigger 7636. In some embodiments, the device further comprises a latch 7642 and latch spring 7644. The device may further comprise a push rod 7624 that can be moved forward by a spring 7634. The device may comprise a cartridge stop 7626 at the top of the device.

FIG. 76B illustrates the device 7600 with the shot chamber moved forward such that the needle 7606 has penetrated the diaphragm 7610. In some embodiments the cartridge holder 7628 bottoms out on the internal frame 7652. In other embodiments, the cartridge holder 7628 bottoms out through the cartridge 7620 and the cartridge stop 7626. In some embodiments, the trigger paddle 7640 released the trigger latch 7642. In other embodiments, the trigger 7636 release the trigger latch 7642 directly. In some embodiments the device features an internal release 7650 that separates the motions of the push rod 7624 from the cartridge holder 7628 only when the cartridge holder has fully bottomed out and the needle 7606 penetrates the diaphragm 7610. In some embodiments, the internal release comprises of a compliant clip as part of the cartridge holder 7628. In some embodiments, the compliant clip may be part of the push rod 7624. FIG. 76C illustrates the device 7600 wherein the push rod 7624 has moved the plunger 7614 forward so as to deliver a fluid through the dispensing tip 7602. FIG. 76D illustrates the device 7600 wherein a ring 7646 is shown to be oriented and positioned perpendicular to a longitudinal axis of the device 7600. In some embodiments, the ring 7646 features a pin that prevents the cartridge 7620 from unintentionally coming in contact with the needle 7606. In some embodiments, the device 7600 comprises a cap 7648 which included protrusions that prevent the trigger 7636 from being unintentionally depressed.

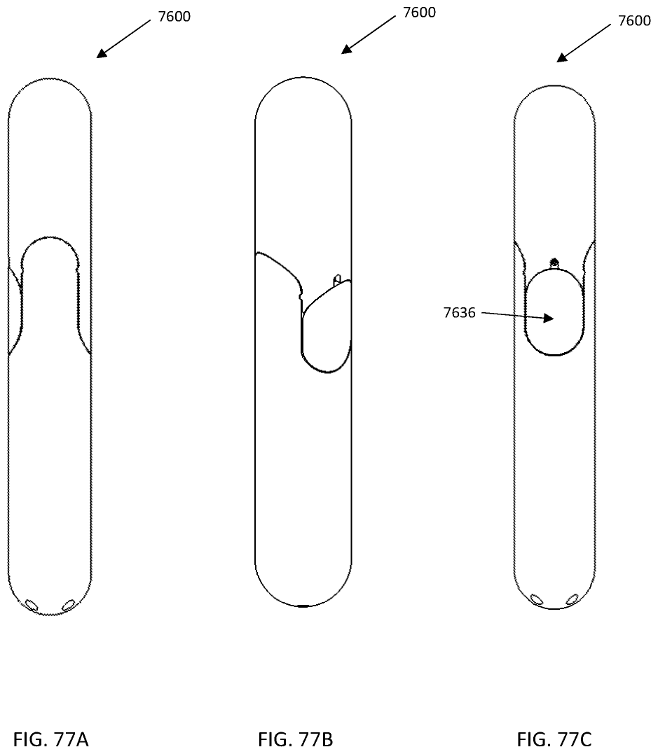

FIG. 77A illustrates a side view of the device 7600. FIG. 77B illustrates a side perspective view of the device 7600. FIG. 77C illustrates a front view of the device 7600, with a trigger 7636 visible.

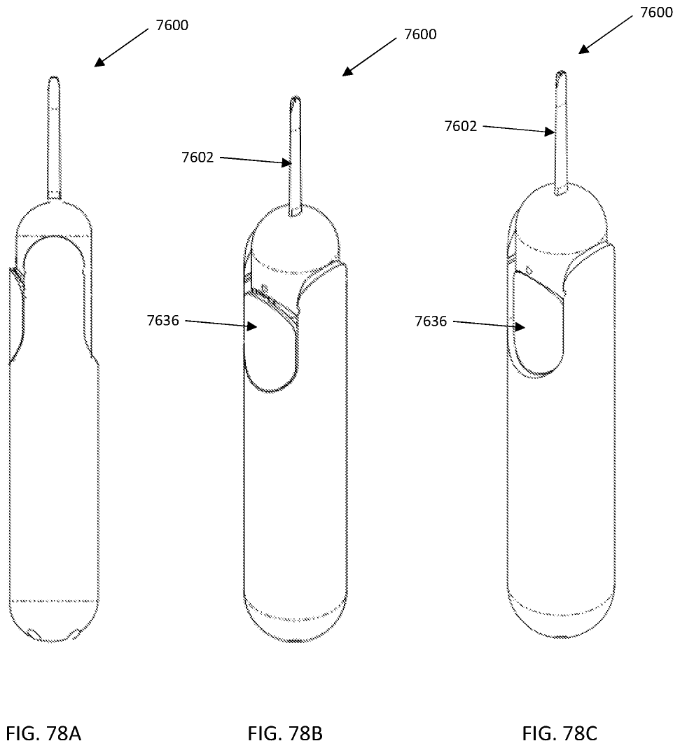

FIG. 78A illustrates a side view of the device 7600. FIG without a cap, thereby exposing the dispensing tip 7602. FIG. 78B illustrates a side perspective view of the device 7600 without a cap, while FIG. 78C illustrates a side perspective view of the device 7600 with the trigger 7636 pushed in. In some embodiments, the top portion of the device is to act as a nose pillow to prevent inserting the cannula too far. In some embodiments, the top portion of the device 7600 is adjustable to either increase or decrease the depth of insertion of the cannula 7602.

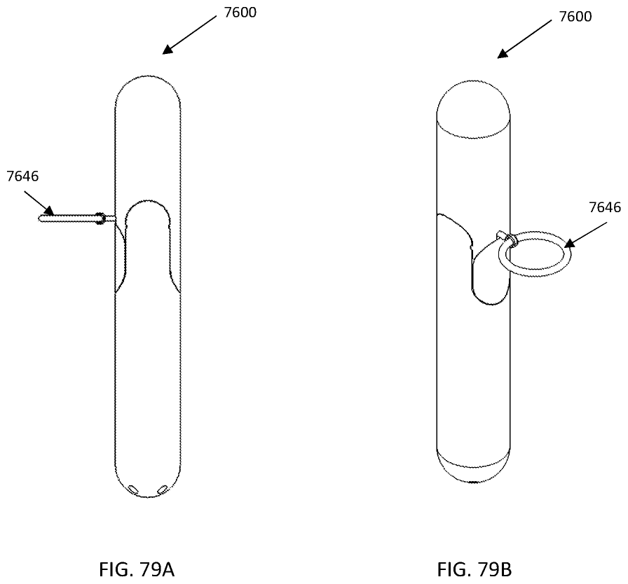

FIGS. 79A-B illustrates a side view and side perspective view of the device 7600 respectively, wherein the ring 7646 is shown to be oriented and positioned perpendicular to a longitudinal axis of the device.

In some embodiments, an intranasal drug delivery device as depicted in FIG. 11 is provided with a two-stage triggering mechanism wherein the needle and dispensing tip are manually pushed towards the cartridge, such that the needle pierces the diaphragm. The actuator is then actuated, such that push rod engages with the plunger to push the fluid within the shot chamber through the dispensing tip and into the nasal cavity or olfactory region of the patient.

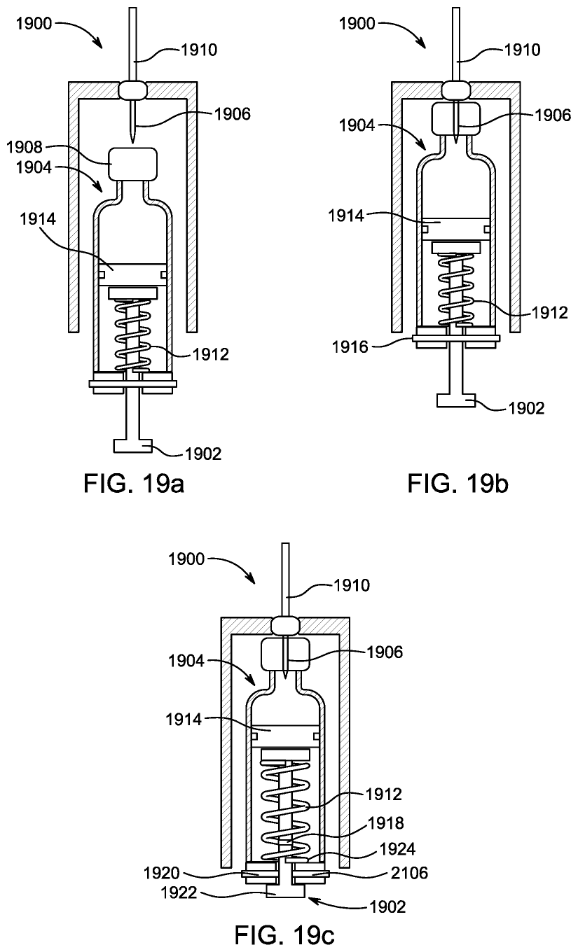

FIGS. 19 a-c show an example intranasal drug delivery device 1900 according to some embodiments, wherein a two-stage triggering mechanism is executed with a single button push.

When actuator 1902 is first pushed by a user, the cartridge 1904 is pressed into a needle 1906. The needle 1906 pierces the diaphragm 1908 (i.e. the cartridge septum) and opens a fluid path through the channel 1910 (cannula) as shown in FIG. 19b. Actuator 1902 is connected directly to plunger 1914. When the actuator 1902 is pressed a second time by a user, spring 1912 releases and depresses the plunger 1914, ejecting fluid through the channel 1910 as shown in FIG. 19c.

Spring 1912 may be released by breaking a shear pin 1916 into pieces 1918 and 1920, as shown in FIGS. 19b and 19c. In other embodiments the spring 1912 may be released when injection molded breakoff points or wings snap off of the plunger 1914. In other embodiments the spring 1912 may be released by a ball detent mechanism, molded snap fit component or other mechanism that is activated by reaching a pre-set force. In still other embodiments the spring 1912 may be released by the press force separating a magnet in the plunger from a magnet in the system body.

The travel of plunger 1914 is limited by a stop mechanism 1904 to set a total dose. Stop mechanism may comprise actuator projections 1922 that engage the base of the cartridge 1924.

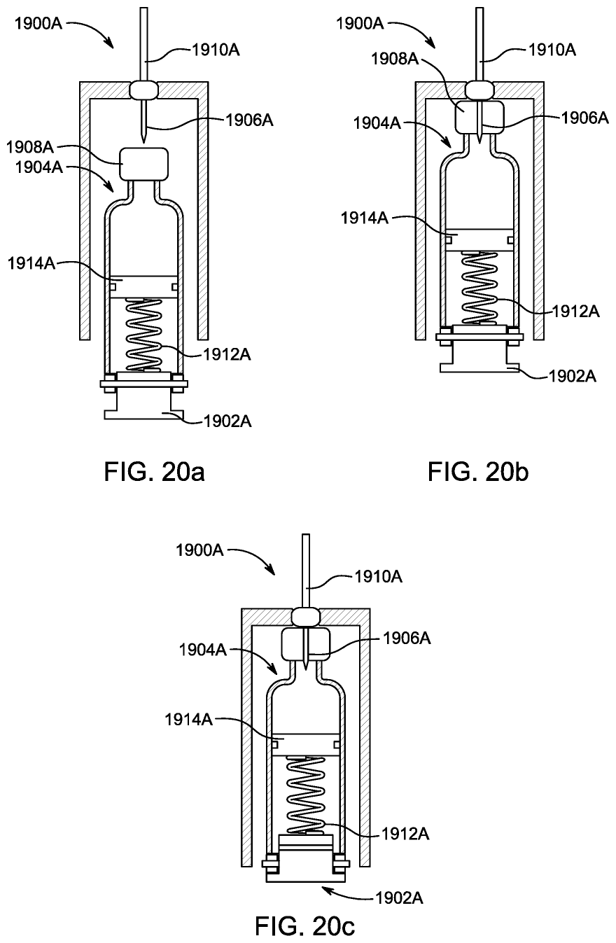

FIGS. 20 a-c show an example intranasal drug delivery device 1900A according to some embodiments, wherein a two-stage triggering mechanism is executed with a single pushing motion. In this embodiment, the actuator 1902A is connected to spring 1912A, which is connected to plunger 1914A. After actuator 1902A is pushed by a user, the cartridge 1904A is pressed into a needle 1906A and the needle 1906A pierces the diaphragm 1908A and opens a fluid path through the channel 1910A (cannula) as shown in FIG. 20b, wherein as the user continues to push on the actuator 1902A, this builds up spring force in the user's hand (or other method used to press the button). When sufficient spring force is achieved, the actuator 1902A is released. The actuator 1902A may be released by several different methods, as described above. The spring force built up behind the actuator 1902A then rapidly compresses the spring 1912A between the actuator 1902A and the plunger 1914A. The spring 1912A then dispenses the fluid from the channel 1910A.

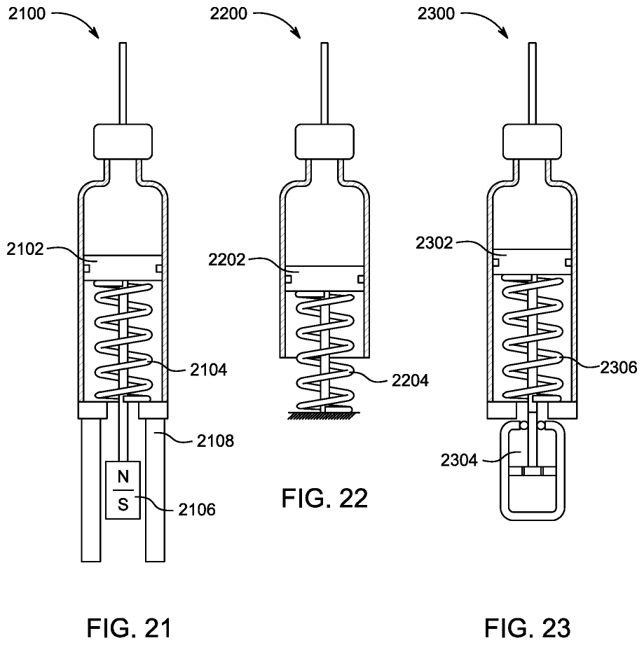

In some embodiments, the device comprises a damping mechanism, examples of which are described further below with reference to FIGS. 21-33. Elements such as the dispensing tip, the needle that pierces the diaphragm, and an outer body are not shown in all views, but may be included in some embodiments. In each of these example embodiments, the device 2100/2200/2300/2400/2500/2600/2700/2800/2900/3000/3100/3200/3300 is configured to eject a jet of fluid through a channel with a controlled velocity profile. This assists in preventing excessive shear on the delivered drug, some of which may be damaged by shear. For example, in some embodiments the device is configured to eject a jet of fluid starting at a high initial velocity but dropping linearly to a near zero jet velocity at the end of j et dispensing.

FIG. 21 shows an example device 2100 according to some embodiments, wherein a plunger 2102 is pushed by a spring 2104. In the FIG. 21 embodiment, the velocity of the plunger 2102 is controlled by an eddy current brake connected to the traveling end of the spring 2104. In the FIG. 21 embodiment, the damping mechanism comprises a magnet 2106 connected to the plunger 2102 moves through a conductive jacket 2108, generating eddy currents and limiting the maximum plunger speed. In another embodiment the velocity of the plunger 2102 may be controlled by having magnet 2106 spun by a helix on a shaft connected to the traveling end of the spring (not shown). By changing the geometry of the conductive jacket to generate more or less eddy current at different locations along the plunger travel, the velocity profile for the ejected payload can be controlled.

FIG. 22 shows an example device 2200 according to some embodiments, wherein the velocity of the plunger 2202 travel is controlled by a damping mechanism inherently formed by the construction of the device 2200 and the materials chosen. For example, in some embodiments part tolerances and material variations are controlled to provide a plunger 2202 friction and spring 2204 K value configured to ensure desired jet velocity profile.

FIG. 23 shows an example device 2300 according to some embodiments, wherein the velocity of the plunger 2302 is controlled by a damping mechanism comprising a viscous damper 2304 connected to the traveling end of the spring 2306. The damper 2304 is filled with air or with viscous liquid (e.g. oil). The damper 2304 controls the velocity of the traveling end of the spring 2306. Maximum velocity is limited by the damper 2304, and as the spring 2306 extends, it's driving force decreases. This provides an initially high velocity followed by a decrease in velocity over the total dispensed volume. It may also provide a constant velocity over the total dispensing.

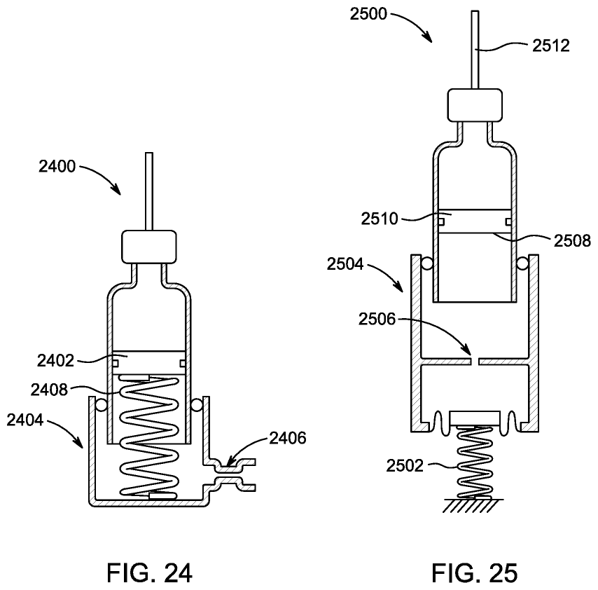

FIG. 24 shows an example device 2400 according to some embodiments, wherein the velocity of the plunger 2402 is controlled by a damping mechanism comprising a sealed chamber 2404 attached to the back of the device 2400 connected to a spring 2408, which is connected to the plunger 2402. Air must be drawn into the chamber 2404 to allow the plunger 2402 to advance, but air flow into the chamber 2404 is limited by ether 1) a flow control valve (not shown) or 2) a simple flow restriction 2406 (e.g. narrow channel, orifice plate).

FIG. 25 shows an example device 2500 according to some embodiments, wherein the damping mechanism comprises a spring 2502 used to compress a body of air (e.g. pushing on a bellows, pushing on a diaphragm, pushing a piston) into a sealed chamber 2504. The compressed air flow through a flow restriction 2506 that controls air flow rate to the device 2500. The outside of the device 2500 body seals to the sealed chamber 2504 (e.g. O-ring seal). The air then pushes on the back side 2508 of the piston 2510, pushing the drug out of the channel 2512. Because the flow rate of air is controlled by the flow restriction 2506, the rate of travel for the piston 2510 is controlled. The flow restriction 2506 may be simple, like an orifice plate, narrow tube, or narrow drilled hole, but it may also be a pneumatic device like a pressure relief valve, or flow control valve.

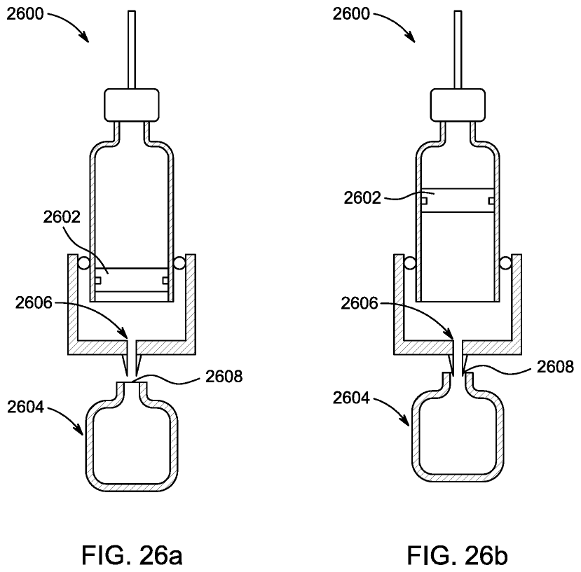

FIGS. 26 a-b show an example device 2600 according to some embodiments, wherein control over the velocity of the plunger 2602 is be achieved by a damping mechanism comprising a container 2604 of compressed gas (e.g. CO2 canister, sealed canister of air, N2, etc.). The container 2604 of compressed gas is connected to the flow restriction 2606 by piercing a membrane 2608 or septum or by connecting with a valve. A leak point may be added to the chamber to cause pressure applied to the device 2600 to dissipate over time. This provides a decreasing velocity profile for the fluid jet. The compressed gas container may be connected to the device 2600 chamber by piercing a membrane on the canister, by a valve, or by a similar mechanism.

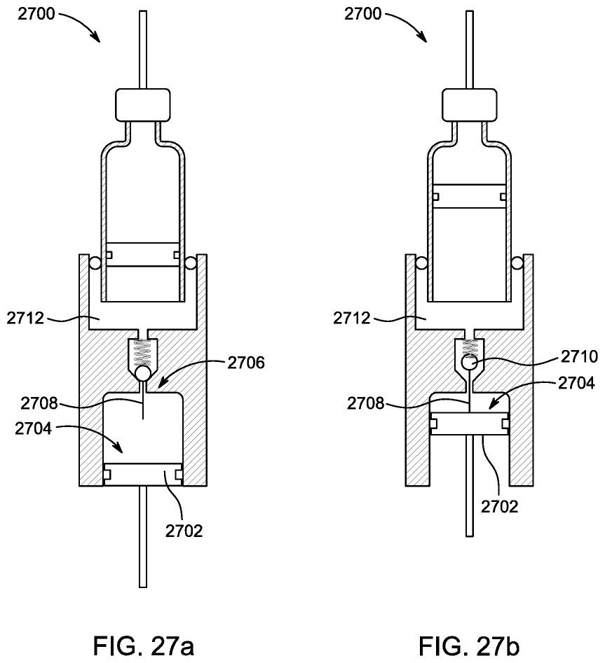

FIGS. 27 a-b show an example device 2700 according to some embodiments, wherein the damping mechanism comprises a piston 2702, sealed chamber 2704, pin and ball valve 2706. In this embodiment, the piston 2702 is moved and compressed gas in sealed chamber 2704 is provided instantaneously using a mechanically operated valve such as pin and ball valve 2706. When the piston 2702 reaches the top of the chamber 2704, a pin 2708 is pushed by the piston 2702, opening a ball valve 2710 to release pressure into the shot chamber 2712.

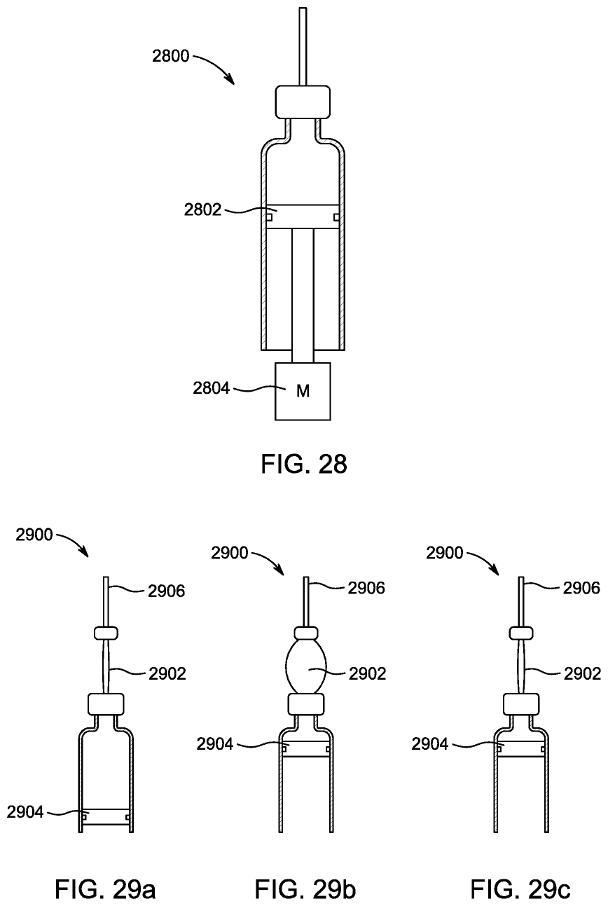

FIG. 28 shows an example device 2800 according to some embodiments, wherein a plunger 2802 is pushed by an electric motor 2804 (e.g. stepper motor, DC motor, brushless motor, etc.) which provides the function of both actuating force and a damping mechanism. Circuitry onboard the electric motor 2804 controls the plunger 2802 velocity to set the desired ejected fluid velocity profile. Control of the electric motor 2804 may be open loop or closed loop. Motor 2804 may be a liner motor, or a rotary motor combined with gearing, a linkage, cam, lead screw, or other mechanical element to drive the plunger 2802.

FIGS. 29 a-c show an example device 2900 according to some embodiments, wherein controlled jet velocity is provided by a damping mechanism comprising an elastomeric chamber 2902. This occurs in two steps. First, the plunger 2904 is depressed to fill the elastomeric chamber 2902, as shown in FIG. 29b. Second the fluid path to the channel 2906 is opened, now spring force stored in the stretched elastomeric chamber 2902 forces the fluid out of the channel 2906 as shown in FIG. 29c.

The flow resistance of the fluid path out of the elastomeric chamber 2902 is matched to the stiffness of the elastomeric chamber 2902 to provide a controlled jet velocity profile. As the elastomeric chamber 2902 relaxes, the pressure on the fluid decreases, so this provides an initial high velocity followed by a decrease in jet velocity.

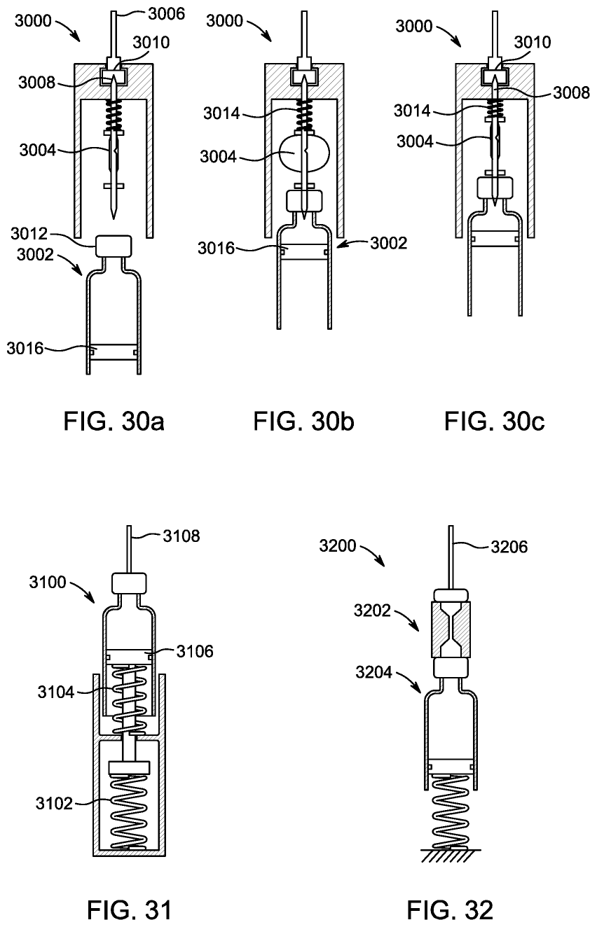

FIGS. 30 a-c show an example device 3000 according to some embodiments, wherein a cartridge 3002 is depressed to fill the elastomeric chamber 3004 and the fluid path to channel 3006 is opened with a single motion. In this embodiment, a needle 3008 is partially embedded in a septum 3010 to seal the end of the needle 3008, as shown in FIG. 30a. First, as the plunger 3016 moves, the diaphragm 3012 is pierced. As the plunger 3016 continues to move as shown in FIG. 30b, the elastomeric chamber 3004 is loaded with fluid. The spring 3014 prevents travel of the cartridge 3003 until the plunger 3016 is sufficiently depressed. Third, the plunger 3016 travel ends, the spring 3014 is compressed, and the septum 3010 is pierced by needle 3008 as shown in FIG. 30c. Fourth, the elastomeric chamber 3004 forces fluid out through the channel 3006. As the elastic elastomeric chamber 3004, pressure drops, providing a decreasing velocity profile. Chamber geometry can be varied to make a linear or non-linear decreasing velocity profile.

FIG. 31 shows an example device 3100 according to some embodiments, wherein a large spring 3102 with a limited initial travel is used to break static friction in the piston 3106 and a second spring 3104 provides the force to fully dispense the drug. Large spring 3102 is a higher force spring than second spring 3104. The flow path out of the channel 3108 is long enough that the high velocity travel from the large spring 3102 does not cause fluid to leave the channel 3108.

FIG. 32 shows an example device 3200 according to some embodiments, wherein the flow rate of the jet is controlled by a flow restriction device 3202 between a cartridge 3204 and a channel 3206. The flow restriction device 3202 can be long and gradual to keep a laminar flow profile. This will prevent excessive shear on the delivered drug (e.g. protecting the viability of vaccines). The flow restriction device 3202 could also be more compact but producing a turbulent flow. The flow restriction could also be an orifice plate. This would make a more compact device suitable for delivering robust therapeutic agents. The flow restriction device 3202 could also be replaced by an active element like a constant velocity flow control valve, a pressure relief valve, or a pressure control valve.

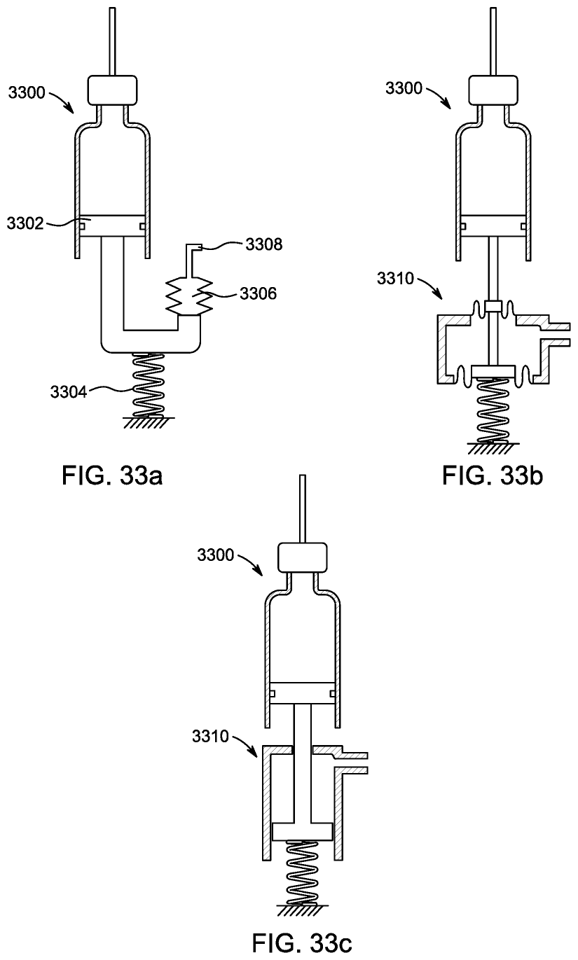

FIG. 33 a-c show an example device 3300 according to some embodiments, wherein the plunger 3302 is driven by a spring 3304, but piston velocity is controlled by bellows 3306 filled with air. As the piston 3302 travels up, the bellows 3306 are compressed, and air is forced through a flow restriction 3308 (e.g. simple orifice plate, small drilled hole, pressure control valve, flow rate control valve). The rate that the bellows 3306 can deform is controlled by the rate of air flow through the flow restriction 3308. This could be accomplished by an arrangement where air is contained in a diaphragm 3310, rolling diaphragm or a piston as shown in FIGS. 33b and 33c. It may also be accomplished in the same configuration shown in FIG. 33a but with a diaphragm, rolling diaphragm, or piston.

Air may vent externally to the device, or it may vent into a secondary chamber to avoid the need for an external vent.

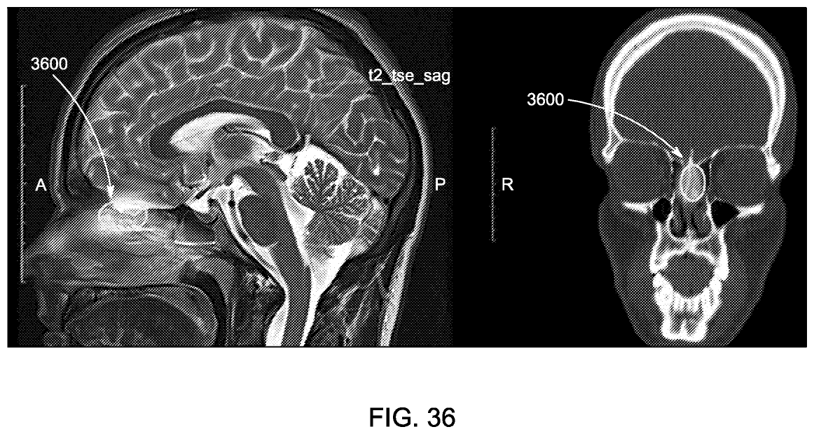

A prototype device including a cannula and damping mechanism has been tested to demonstrate targeted delivery of the fluid. The testing comprised inserting the cannula into the upper nares of a patient and ejecting a liquid jet or stream of fluid through the cannula. In the testing, technicium 99 was used as a tracer fluid. A scan of the patient performed following the delivery of the fluid show that the fluid is deposited at the olfactory region of the patient 3600, as shown in FIG. 36. The presence of the technicium 99 appears as a light region on the scan shown in FIG. 36.

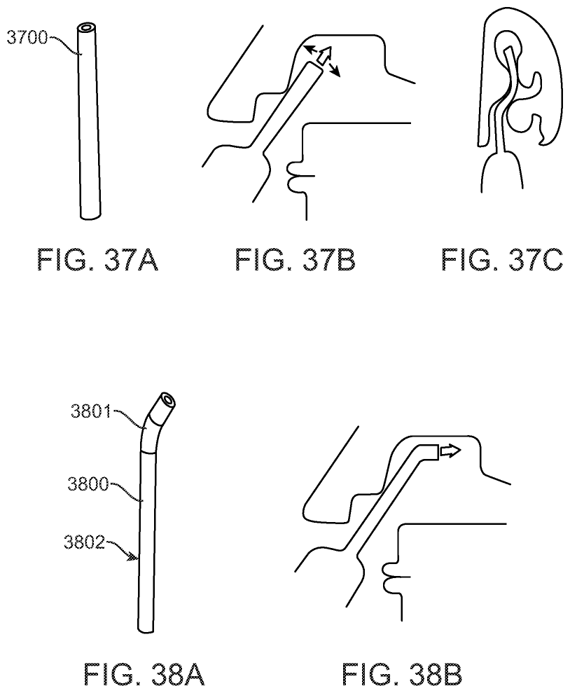

FIG. 37A shows an exemplary embodiment of the dispensing tip 3700 used with an intranasal drug delivery device described herein. In some embodiments, the tip is flexible and has a circular cross-section. In some embodiments, the tip is flexible and has a non-circular cross-section. In this embodiment (FIG. 37A), the tip has an elliptical cross section to provide stiffness in the plane of the major axis (of the elliptical shape) while maintaining flexibility in the plane of the minor axis. In some embodiments, the tip is inserted with additional stiffness in the sagittal plane (e.g., along the major axis of an elliptical cross-section), so it can maintain its shape and stay aligned with the target site (FIG. 37B). However, the tip's flexible plane (e.g., along the minor axis of an elliptical cross-section) allows the tip to conform to patient anatomy perpendicular to the sagittal plane (FIG. 37C)

FIG. 38A shows an exemplary embodiment of the dispensing tip 3800 used with an intranasal drug delivery device described herein. In some embodiments, the tip 3800 includes a bent section 3801 to direct an ejected drug away from a longitudinal axis of a straight section 3802 of the dispensing tip. This allows a more vertical insertion of the tip while still directing a drug ejection toward a target site (FIG. 38B) within a patient's nasal cavity. As described herein, for any embodiment, delivery of a drug may comprise delivery of a fluid. In some embodiments, the fluid comprises a liquid, a gel, a powder, or any combinations thereof. In some embodiments, the drug comprises a powder suspended in a liquid or gaseous fluid. In some embodiments, the drug comprises a powder that is delivered by the device.

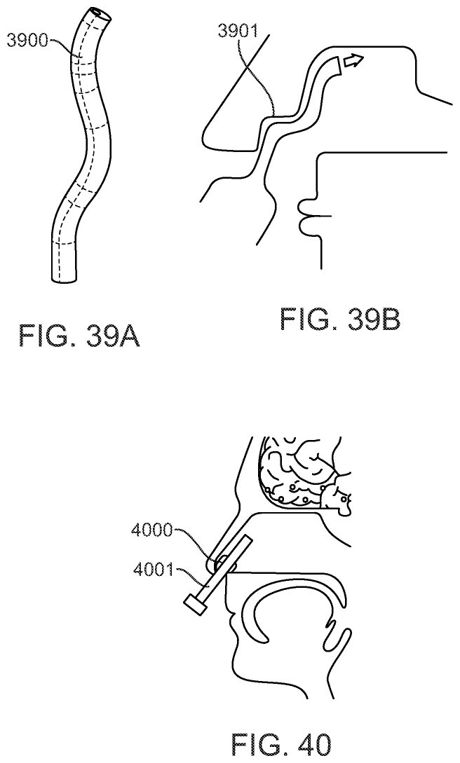

FIG. 39A shows an exemplary embodiment of the dispensing tip 3900 used with an intranasal drug delivery device described herein. In some embodiments, the tip 3900 is shaped with several curves to conform to the shape of the patient's nasal valve 3901 while still directing drug ejection to a target site. In some embodiments, the tip 3900 may conform to the nasal valve with flexible sections.

FIG. 40 shows a possible accessory for a dispensing tip used with an intranasal drug delivery device described herein. In some embodiments, the dispensing tip 4001 further comprises a base of compliant material 4000 (e.g. foam, silicone, memory foam). In some embodiments, the base of compliant material 4000 protrudes from a dispensing tip body 4001. In some embodiments, when inserted, the compliant base contacts the nasal valve, which locates the dispensing tip and prevents over insertion of the dispensing tip within the nasal cavity.

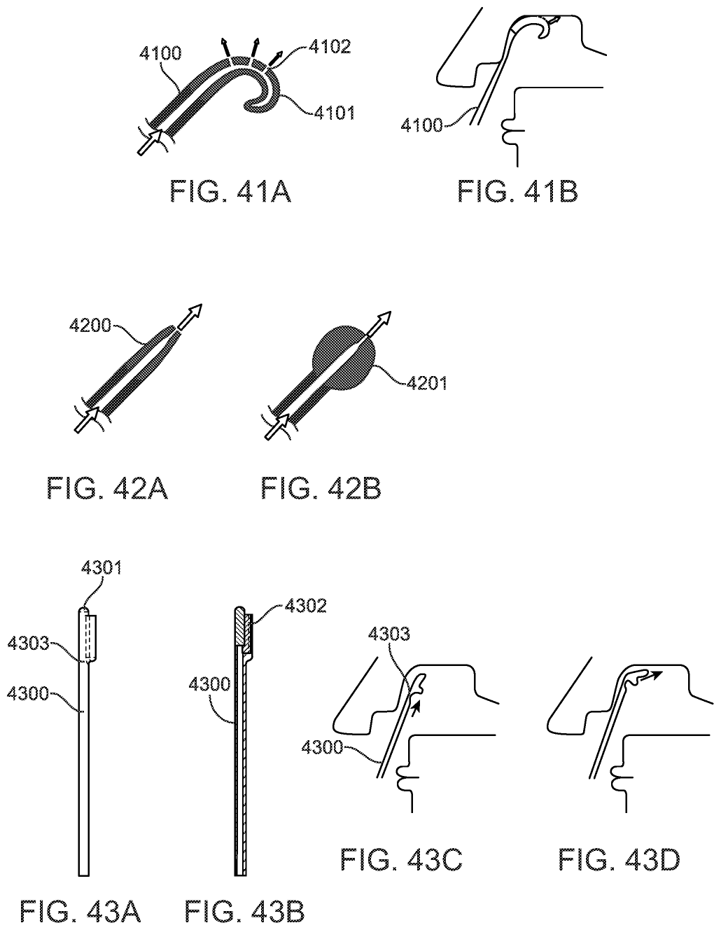

FIG. 41A shows an exemplary embodiment of the dispensing tip 4100 used with an intranasal drug delivery device described herein. In some embodiments, the tip 4101 is curled over, and the curled section of the tip includes one (or more) perforations 4102. In some embodiments, the curved shape aids in insertion and orientation within the nasal cavity. In some embodiments, the tip 4100 is made from a soft material, such that the tip could be placed in contact with a target site within a patient's nasal cavity as shown in FIG. 41B and used to gently dispense the drug directly to the target site.

FIGS. 42A-B show additional exemplary embodiments of the dispensing tip used with an intranasal drug delivery device described herein. In some embodiments, the dispensing tip comprises a soft end 4200 or a ball end 4201 to prevent accidental damage to the patient and to improve patient comfort on insertion.

FIGS. 43A-B shows an exemplary embodiment of the dispensing tip 4300 used with an intranasal drug delivery device described herein. In some embodiments, the end of the dispensing tip includes a rounded end 4301, an off-center drug dispensing channel 4302, and a flexible section 4303. When inserted, the rounded end 4301 contacts a surface within a patient's nasal cavity (FIG. 43B) and the tip 4300 flexes (via flexible section 4303) to align the drug dispensing channel 4302 with a target site within the patient's nasal cavity (FIG. 43C).

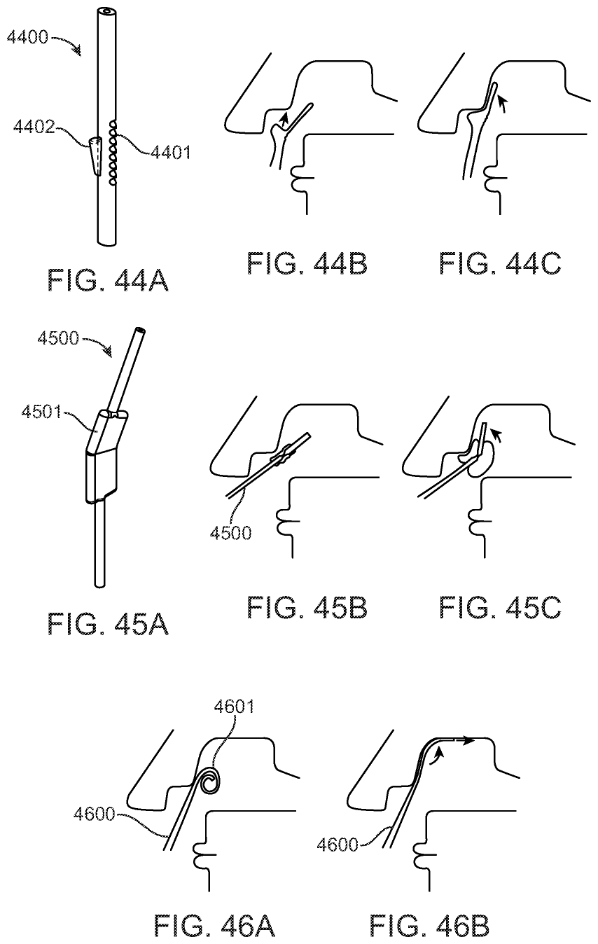

FIG. 44A shows an exemplary embodiment of the dispensing tip 4400 used with an intranasal drug delivery device described herein. In some embodiments, the tip includes a flexible section 4401 and a soft protruding element 4402. When inserted (FIG. 44B), the protruding element contacts the nasal valve of a patient. As the tip 4400 is further inserted, the flexible section 4401 bends to point the tip at the target site (FIG. 44c).

FIG. 45A shows an exemplary embodiment of the dispensing tip 4500 used with an intranasal drug delivery device described herein. In some embodiments, the dispensing tip includes a balloon 4501. After insertion (FIG. 45B), the balloon is inflated (FIG. 45C). When inflated, the balloon conforms to the patient's unique anatomy and points the tip towards a target site within a patient's nasal cavity. The balloon may also be used to open up the nasal cavity to aid in drug dispensing.

FIG. 46A shows an exemplary embodiment of the dispensing tip 4600 used with an intranasal drug delivery device described herein. The tip has a curled over tip 4601. In some embodiments, when a drug is forced into the tip 4600, fluid pressure causes the curled over portion 4601 of the tip to unroll gently within a patients' nasal cavity (FIG. 46B). In some embodiments, when completely unrolled (FIG. 46B), the tip opens under pressure and the drug is dispensed to a target site with the patient's nasal cavity.

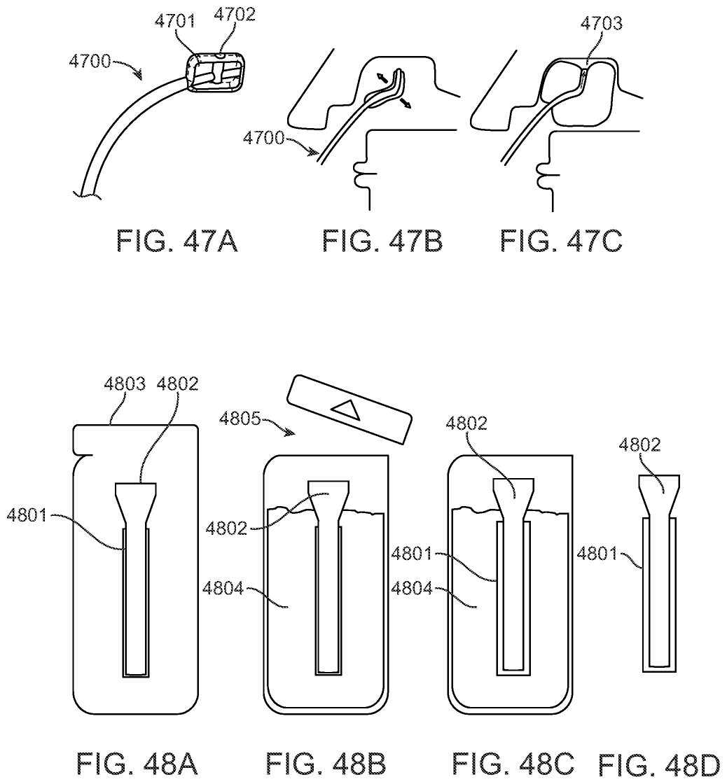

FIG. 47A shows an example embodiment of the dispensing tip 4700 used with an intranasal drug delivery device described herein. In some embodiments, the dispensing tip 4700 comprises an inflatable balloon 4701 at a terminal end of the dispensing tip 4700 with an exit port 4702 integrated into the balloon 4701. After insertion (FIG. 47B), the balloon 4701 is inflated. When inflated, the balloon 4701 fills at least a portion of the nasal cavity near a target site and creates a sealed chamber around the target site (FIG. 47C). Drug is then dispensed through the integrated exit port 4703 directly onto the target site. This technique provides the opportunity to cause a flow of drug into the target site with positive pressure which can increase the rate of uptake for the drug.

FIGS. 48A-D show an exemplary method for improving patient comfort while inserting a dispensing tip into a patient's nasal cavity. In some embodiments, a hydrophilic coating 4801 is applied to the outer surface of a dispensing tip 4802 used with an intranasal drug delivery device described herein, wherein the dispensing tip is contained in a packaging 4803 (FIG. 48A). When this coating 4801 is activated by contact with a hydrating medium (e.g., water, a gel), the coating 4801 activates to become a low friction surface that aids in insertion into a patient's nasal cavity. The most common form of this product is where a sterile, individually packaged single or multi use dispensing tip is packaged in a dry state or condition (FIG. 48A). The user opens 4805 the package and fills the package with a hydrating medium 4804 (FIG. 48bB). After waiting an appropriate time (e.g. 30 seconds), the coating 4801 is activated (FIG. 48C). The user then removes the dispensing tip 4802 from the package, and the tip is ready for insertion (FIG. 48D). Rather than filling the package with hydrating medium, the user may provide a container of hydrating medium, remove the dispensing tip from the package, and place the dispensing tip into the container of hydrating medium. The user submerges the tip in hydrating medium, waits for the hydrophilic coating 4801 to activate, and then removes the tip for use (FIG. 48D)

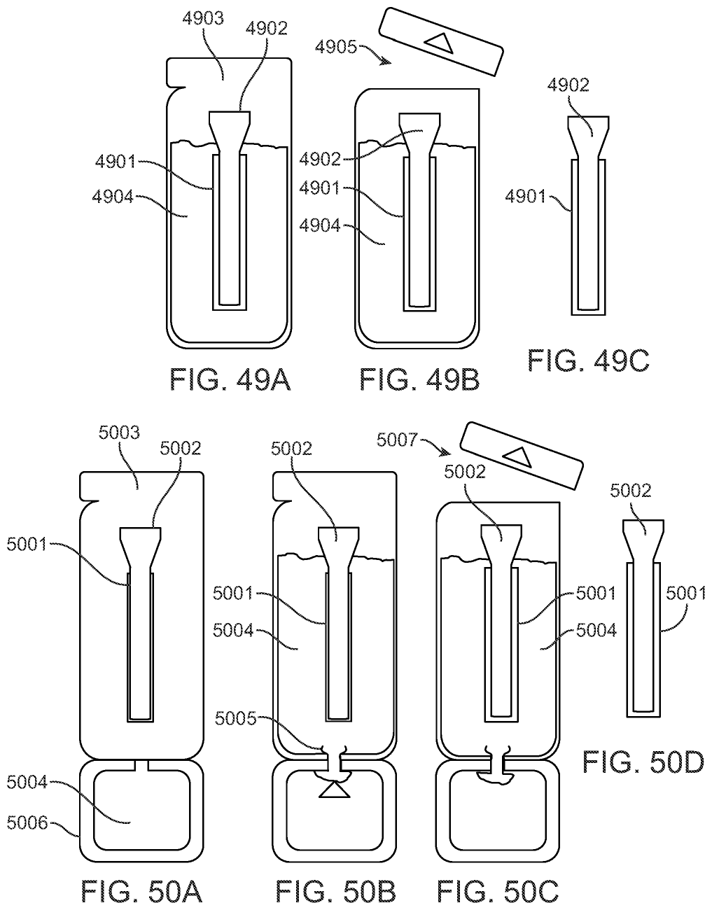

FIGS. 49A-C show an exemplary method for improving patient comfort while inserting a dispensing tip into a patient's nasal cavity 4900. In some embodiments, a hydrophilic coating 4901 is applied to the outer surface of a dispensing tip 4902 used with an intranasal drug delivery device described herein, wherein the dispensing tip is contained in a package 4903 (FIG. 49A). The dispensing tip is submerged in a hydrating medium 4904 (e.g., water, a gel) within the package 4903. In some embodiments, the hydrophilic coating 4901 is allowed to activate during manufacture/transport so that the dispensing tip 4902 is ready for use before the package is given to the user. The user opens 4905 the package (FIG. 49B) and removes the dispensing tip 4902 ready for use (FIG. 49C).

FIGS. 50A-D shows an exemplary method for improving patient comfort while inserting a dispensing tip into the nose 5000. In some embodiments, a hydrophilic coating 5001 is applied to the outer surface of the dispensing tip 5002 used with an intranasal drug delivery device described herein, wherein the dispensing tip is contained in a package 5003 (FIG. 50A). In some embodiments, the package further comprises a separated compartment 5006 that contains a sufficient amount of hydrating medium 5004 (e.g., water, a gel) to activate the hydrophilic coating 5001. In some embodiments, the separated compartment 5006 is a different compartment from a compartment containing the dispensing tip 5002. In some embodiments, the separated compartment 5006 is configured to be opened, so as to allow the hydrating medium 5004 to cover the hydrophilic coated surface 5001 (FIG. 50B). In this embodiment, a membrane separating the separated compartment 5006 from the compartment containing the dispensing tip 5002 is burst 5005 (e.g., via pressure build-up within the compartment 5006 by a user pressing the compartment 5006). In some embodiments, the user continues to press on the compartment 5006 until the dispensing tip 5002 is at least partially submerged in the hydrating medium 5004. Depending on the product, and on the amount of hydrating medium in the separate compartment 5006, the package may be manipulated to fully cover the dispensing tip 5002 in the hydrating medium 5004. After waiting an appropriate time (e.g. 30 seconds) after submerging the dispensing tip 5002 at least partially in the hydrating medium 5002, the hydrophilic coating 5001 is activated. In some embodiments, the package 5003 (FIG. 50C) is opened 5007. In some embodiments, the dispensing tip 5002 is removed from the package, and the tip 5002 is ready for use (FIG. 50D). In some embodiments, the package may include features to prevent the hydrating medium 5004 from flowing back into the compartment 5006 (e.g. the compartment 5006 may be configured to be rolled up and held in place, the compartment 5006 may have a one way valve, the upper and lower half of the compartment 5006 may snap together, or the compartments 5006 may be separated from the compartment containing the dispensing tip by a clamp)

In some embodiments, for any of the methods disclosed in FIGS. 48A to 50D, the hydrating medium may be a gel or a viscous liquid that is less likely to spill when handling the package (e.g., handling the dispensing tip and removing the dispensing tip from the package) than a liquid hydrating medium like water.

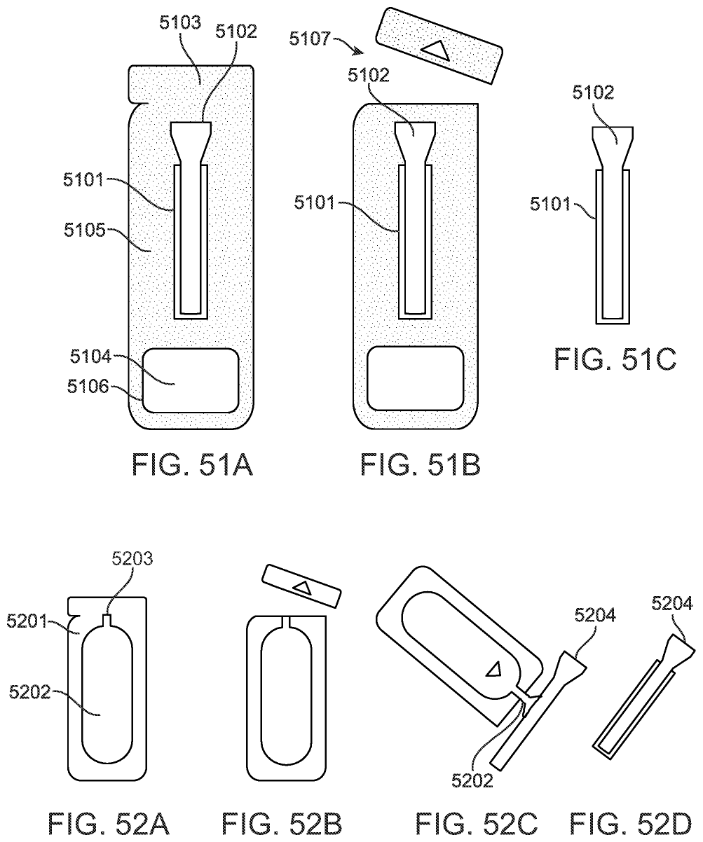

FIGS. 51A-D show an exemplary method for improving patient comfort while inserting a dispensing tip into a patient's nasal cavity, wherein the method reduces the risk of spilling a hydrating medium used to activate a hydrophilic coating. Here, the hydrophilic coating 5101 is activated by a hydrating vapor 5105 (e.g. water vapor) rather than by liquid or gel. A hydrophilic coating 5101 is applied to the outer surface of the dispensing tip 5102 used with an intranasal drug delivery device described herein, and the dispensing tip is contained in a package 5103. The package contains a body of a hydrating medium 5104 (e.g., water, gel) that is contained within a piece of foam 5106. In some embodiments, the foam 5106 prevents loose liquid from presenting a spill hazard. The hydrating medium 5104 may also be contained in a piece of fabric, a porous plastic body, a porous ceramic body, or a similar wicking body. The hydrating medium 5104 may also be contained in a solid material that is saturated with the hydrating medium 5104 (e.g. nylon plastic soaked in water). The hydrating medium 5104 may also be a gel. The hydrating medium 5104 may also be placed in a separate pocket within the package that is separated from the dispensing tip by a vapour permeable but hydrating medium impermeable membrane. The hydrating medium 5104 may also be contained within a separate tortuous chamber that is directly connected to a chamber containing dispensing tip 5102. The tortuous path minimizes the chance of spilling the hydrating medium 5104. During manufacture or transport, the package 5103 is left closed long enough such that the package 5103 fills with hydrating vapor 5105, the vapor activates the hydrophilic coating 5101, and the dispensing tip 5102 becomes ready for use before the package reaches a user (FIG. 51A). The package (FIG. 51B) is then opened 5107, wherein the dispensing tip 5102 is removed and ready for use (FIG. 51C)

FIGS. 52A-D show an exemplary method for improving patient comfort while inserting a dispensing tip into a patient's nasal cavity. In some embodiments, a user is provided with a package 5201 that contains lubrication gel 5202 and may include a narrow dispensing channel 5203 (FIG. 52A). In some embodiments, the user may also be provided with any other standard package of gel (e.g. blow mold fill ampule, square tear open pouch, etc.). In some embodiments, the user opens the package of gel (FIG. 52B) and applies the lubrication gel 5202 as a coating to the surface of the dispensing tip 5204 used with an intranasal drug delivery device described herein (FIG. 52C). The dispensing tip 5204 is now ready for use (FIG. 52D).

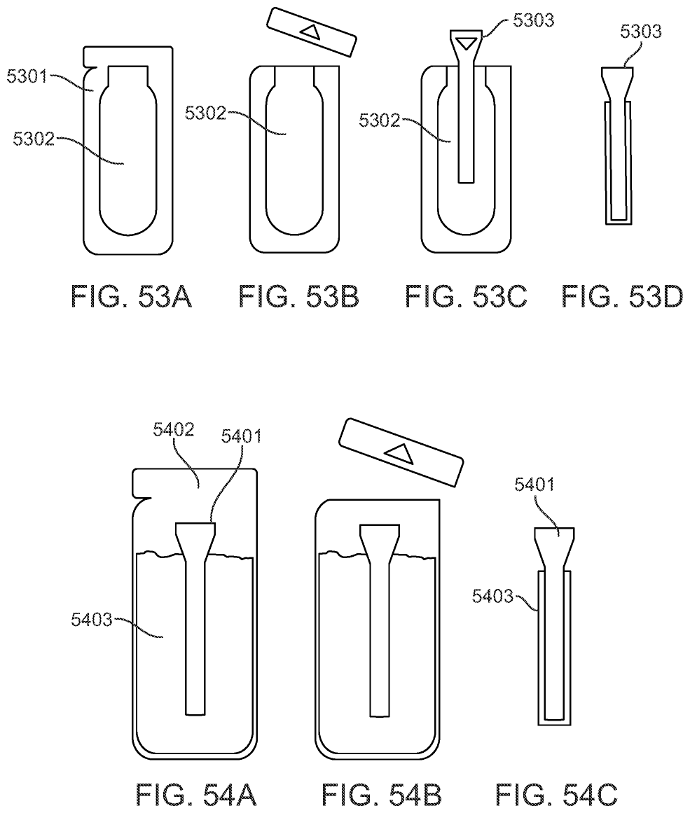

FIGS. 53A-D show an exemplary method for improving patient comfort while inserting a dispensing tip into a patient's nasal cavity. In some embodiments, a user is provided with a package 5301 that contains lubrication gel 5302 (FIG. 53A). In some embodiments, the user opens the package 5301 of the lubrication gel 5302 (FIG. 53B) and dips a dispensing tip 5303 used with an intranasal drug delivery device described herein into the lubrication gel 5302 (FIG. 53C). In some embodiments, the user then removes the dispensing tip 5303 (FIG. 53D) ready for use.

FIGS. 54A-C show an exemplary method for improving patient comfort while inserting a dispensing tip into a patient's nasal cavity. In some embodiments, a dispensing tip 5401 used with an intranasal drug delivery device described herein is contained in a package 5402 that is filled with lubricating gel 5403. In some embodiments, a user opens the package 5402 (FIG. 54B) and removes the dispensing tip 5401 (FIG. 54C) containing a coating of the lubricating gel 5403, ready for use.

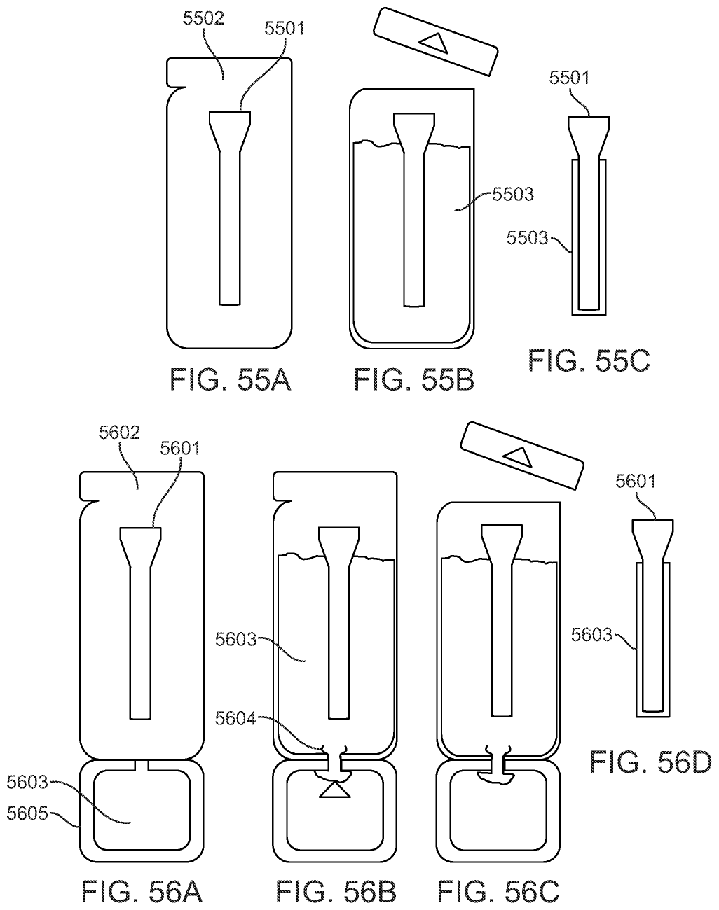

FIGS. 55A-C show an exemplary method for improving patient comfort while inserting a dispensing tip into a patient's nasal cavity. In some embodiments, a dispensing tip 5501 used with an intranasal drug delivery device described herein is contained in a package 5502. In some embodiments, a user opens the package 5502 and fills the package with lubricating gel 5503 (FIG. 55B). The user then removes the dispensing tip 5501 from the package 5502, and the dispensing tip 5501 containing a coating of the lubricating gel 5503 is ready for use (FIG. 55C). In some embodiments, the user may be instructed to manipulate the package 5502 to ensure the dispensing tip 5501 is fully covered in the lubricating gel 5503. The user may be supplied with a separate package (pouch, bottle, etc.) of the lubricating gel, or they may provide their own gel.

FIGS. 56A-D show an exemplary method for improving patient comfort while inserting a dispensing tip into a patient's nasal cavity. In some embodiments, the dispensing tip 5601 used with an intranasal drug delivery device described herein is contained in a package 5602 (FIG. 56A). In some embodiments, the package further comprises a separate compartment 5605 containing a sufficient amount of lubricating gel 5603 to coat the dispensing tip 5601. In some embodiments, the separated compartment 5605 is a different compartment from a compartment containing the dispensing tip 5601. In some embodiments, a user enables for a pathway between the separate compartment 5605 and the compartment containing the dispensing tip 5601 to be opened, allowing a hydrating medium (e.g., lubricating gel 5603) to cover the at least a portion of the dispensing tip (FIG. 56B). In this embodiment, the user presses on the compartment 5605 causing a membrane 5604 to burst. The user continues to press on the compartment 5605 until the dispensing tip is at least partially submerged in the lubricating gel 5603. Depending on the product and on the amount of gel 5603 in the separate chamber 5605, to the package 5602 may be manipulated to fully cover the dispensing tip 5601 in the hydrating medium (e.g., lubricating gel 5603). The user may open the package (FIG. 56C), and then remove the dispensing tip 5601 from the package 5602, wherein the tip 5601 is ready for use (FIG. 56D). The package may include features to prevent the lubricating gel from flowing back into the separate compartment 5605 (e.g. the compartment 5605 may be rolled up and held it in place, the compartment 5605 may have a one way valve, the upper and lower half of the compartment 5605 may snap together, the compartment 5605 may be separated from the compartment containing the dispensing tip 5601 by a clamp).

FIGS. 57 to 61 and FIGS. 42A to 42c illustrate exemplary flow restriction mechanisms that could be used with an intranasal drug delivery device described herein (e.g., a device shown in FIGS. 4, 5, 11, 12, 13, 14 15, 18, 19a to 19c, 20a to 20c, 22, 29a to 29c, 30a to 30c, 31, 32, 34, and 35). In some embodiments, the flow restriction mechanism(s) described herein could also be applied to the flow path of the drug out of devices shown in FIGS. 26a, 26b, 27a, and 27b.

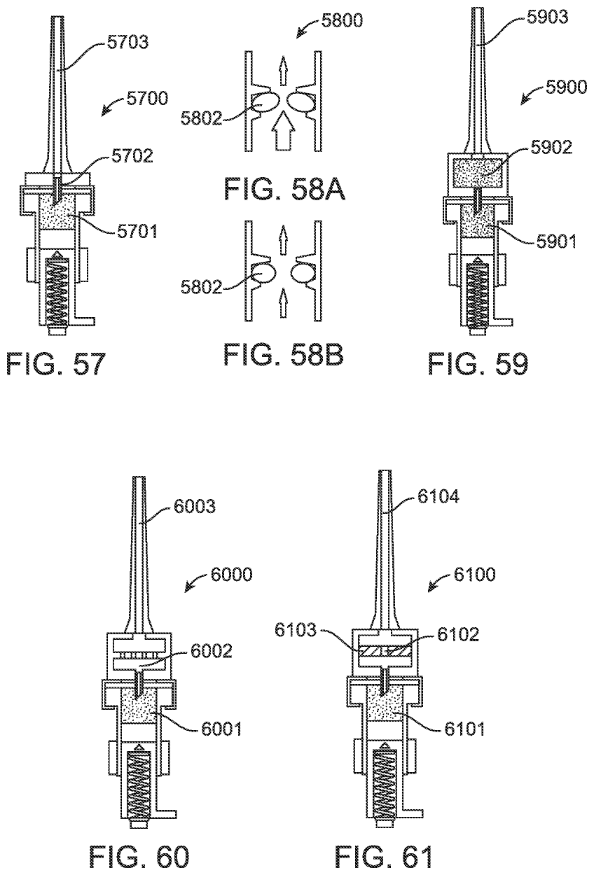

In FIG. 57, in one embodiment of the device 5700, the drug 5701 is forced through the flow restriction 5702 as part of its path out of the dispensing tip 5703. In this device embodiment 3700, the flow restriction 5702 is formed as part of the general architecture of the device 5700. In this device 3700, the flow restriction is a needle piercing a septum to connect the dispensing tip 5703 to the drug container, wherein the flow restriction comprises a narrow inner diameter. The flow restriction 5702 could also be a general flow path molded into the dispensing tip or the device 5700, or it could be an element with a narrow internal pathway added as an insert.

In FIGS. 58A-B, an exemplary flow restriction mechanism is provided, wherein a drug is forced through the flow restriction 5802 as part of its path out of the dispensing tip. In this embodiment, the flow restriction 5802 is a constant flow rate valve. As fluid pressure applied to the flow restriction 5802 increases, it will constrict (FIG. 58A) so as to limit the flow therethrough and maintain a constant or nearly constant flow rate of fluid over a range of pressures. As the fluid pressure reduces, the flow restriction 5802 will allow more the flow therethrough (FIG. 58B). This effect can be used to maintain a constant drug ejection velocity as pressure applied to the flow restriction shifts due to component tolerances and environmental conditions.

In FIG. 59, a drug 5901 is forced through the flow restriction 5902 as part of its path out of the dispensing tip 5903. In this embodiment of the device 5900, the flow restriction 5902 comprises a body of porous media (e.g. ceramic, open cell foam, etc). The porous media provides an advantage over other damping elements by allowing large pressure drops within a small volume, and by providing large pressure drops within a liquid jet or laminar flow regime.

In FIG. 60, a drug 6001 is forced through the flow restriction 6002 as part of its path out of the dispensing tip 6003 of the device 6000. In some embodiments, the flow restriction 6002 is a layer of porous membrane. In some embodiments, the porous membrane allows for a large pressure drop, and producing a diffuse flow over the whole area of the flow channel, which may allow for faster development of liquid jet or laminar flow within the dispensing tip compared to other damping methods (e.g., an orifice plate). Thus, in some embodiments, the porous membrane may result in a more compact device 6000 since a shorter distance of a dispensing tip is needed to achieve laminar flow of a drug exiting the device (if desired).

In FIG. 61, the drug 6101 is forced through the flow restriction 6102 as part of its path out of the dispensing tip 6104 of the device 6100. In this device 6100, the flow restriction 6102 is configured with an orifice plate 6103. The orifice plate flow restriction 6102 allows for large pressure drops over a small distance, wherein the dynamics of an orifice plate are well understood.

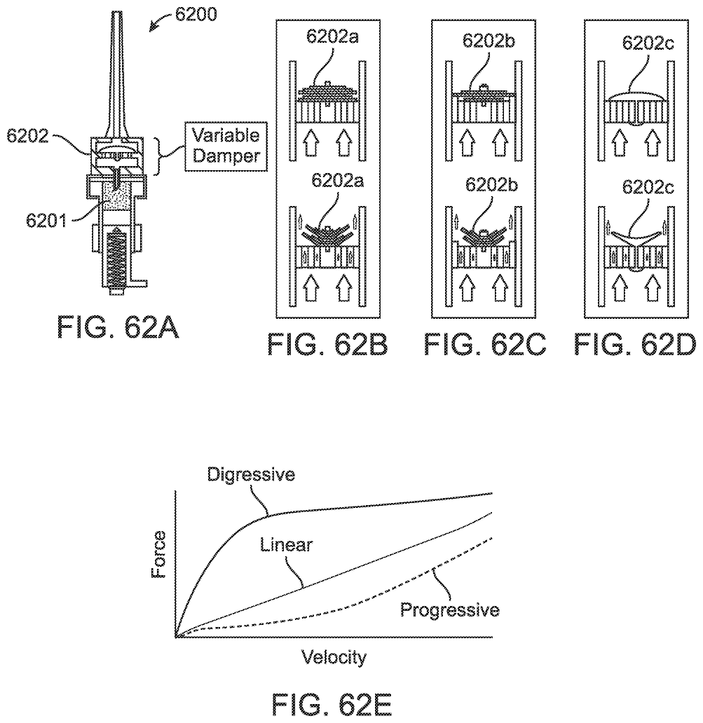

In FIG. 62A, a drug 6201 is forced through the flow restriction 6202 as part of its path out of the dispensing tip of the device 6200. In some embodiments, the flow restriction 6200 is replaced by a first alternate variable flow restriction 6202a (FIG. 62B), a second alternate variable flow restriction 6202b (FIG. 62C), or a third alternate variable flow restriction 6202c (FIG. 62D). In some embodiments, the first alternate variable flow restriction 6202a is constructed from a stack of flexible washers, and it provides a progressive damping response where additional force reduces the flow restriction (top depiction provides a closed position of flow restriction 6202a, and bottom depiction provides open position). In some embodiments, the second alternate variable flow restriction 6202b is constructed from a stack of flexible washers, and it provides a digressive damping response where additional force results in additional flow restriction (top depiction provides a closed position of flow restriction 6202b, and bottom depiction provides open position). In some embodiments, the third alternate variable flow restriction 6202c is constructed as a single molded part and provides a progressive damping response (top depiction provides a closed position of flow restriction 6202c, and bottom depiction provides open position). An exemplary depiction of the relationship between the force applied by progressive and digressive damping and the resulting fluid velocities is shown in FIG. 62C.

Flow restrictions 5700/5800/5900/6000/6100/6200/6202a/6202b/6202c, could also be placed in the air pathway of concepts shown in FIGS. 24, 25, and 33a to 33b to control travel rate of the plunger.

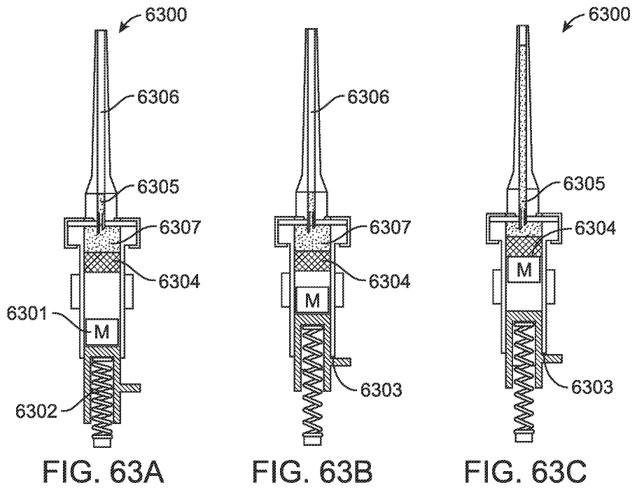

FIG. 63A shows an exemplary intranasal drug delivery device 6300 according to some embodiments. In some embodiments, a mass 6301 is accelerated by spring 6302. In some embodiments, the mass stops accelerating once the spring travel is stopped at a hard stop 6303 (e.g., a protrusion of a casing about a spring that contacts another portion of the device 6300, thereby preventing the spring to continue to move towards the dispensing tip 6306). In some embodiments, the spring 6302 and location of the hard stop 6303 are configured to stop the mass 6301 accelerating at a predetermined velocity. In some embodiments, the mass 6301 strikes a plunger 6304 of a drug container 6304 containing a drug 6307, forcing the drug through a flow restriction 6305 and into the dispensing tip 6307. The flow rate of the drug 6307 out of the device 6300 is determined by the travel rate of the mass 6301 as it decelerates. With low mass and high pressure drop, the mass will experience significant deceleration and the drug will be ejected with a decreasing velocity profile. With a large mass and a low pressure drop, an approximately constant velocity profile of the drug being ejected can be achieved.

FIG. 74 shows an exemplary intranasal drug delivery device 7400 according to some embodiments. In some embodiments, a drug is driven out of the drug container 7401 by a spring 7402 connected to a block of open cell foam 7403. In some embodiments, as the spring dispenses the drug, air trapped within the open cell foam 7403 is forced out of the pores of the foam. In some embodiments, the flow of air through the pores is restricted because of the small size of the pores. In some embodiments, the restricted flow of air provides viscous damping that controls the speed of travel of the drug container plunger and thus controls the flow rate of drug out of the device.

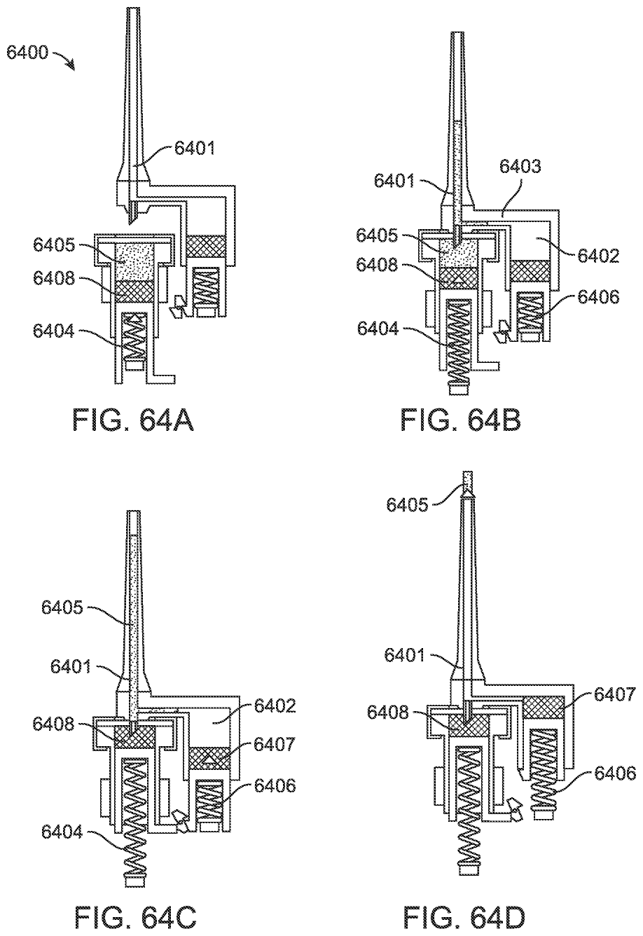

FIGS. 64A-D shows an exemplary intranasal drug delivery device 6400 according to some embodiments. In some embodiments, activation of a first spring 6404 of the device 6400 enables a drug container containing a drug 6405 to be in fluidic communication with a delivery chamber 6401 (e.g. the body of the dispensing tip), wherein the drug is pushed out of the drug container by the first spring 6404 into the delivery chamber 6401 (FIGS. 64A-B). In some embodiments, a second spring 6406 pushes a secondary fluid 6402 (e.g. air) into the delivery chamber 6401 (FIG. 64C) via a second plunger or piston 6407, wherein the secondary fluid is stored in another chamber. In some embodiments, a pump is used to push the secondary fluid 6402 into the delivery chamber 6401 (e.g., a piston pump with piston 6407). In some embodiments, the path for the drug back into the drug container is blocked by a one-way valve, closed valve, or similar feature. In some embodiments, the drug container is a cartridge and the cartridge plunger 6408 closes the path back into the drug container by contacting the needle at the end of its travel. In some embodiments, the drug 6405 is forced out of the dispensing tip by the secondary fluid 6402 by the secondary spring 6406 and/or when a pump is activated (FIG. 64D). In some embodiments, the secondary fluid 6402 (e.g., air) is moved by a spring driven piston pump and the flow rate of the piston pump is controlled by a flow restriction 6403 in the air path to the delivery chamber 6401.

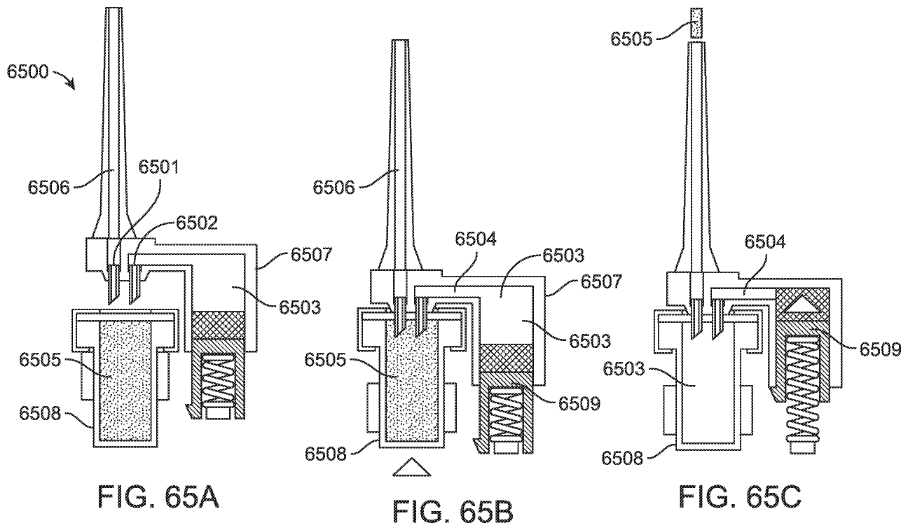

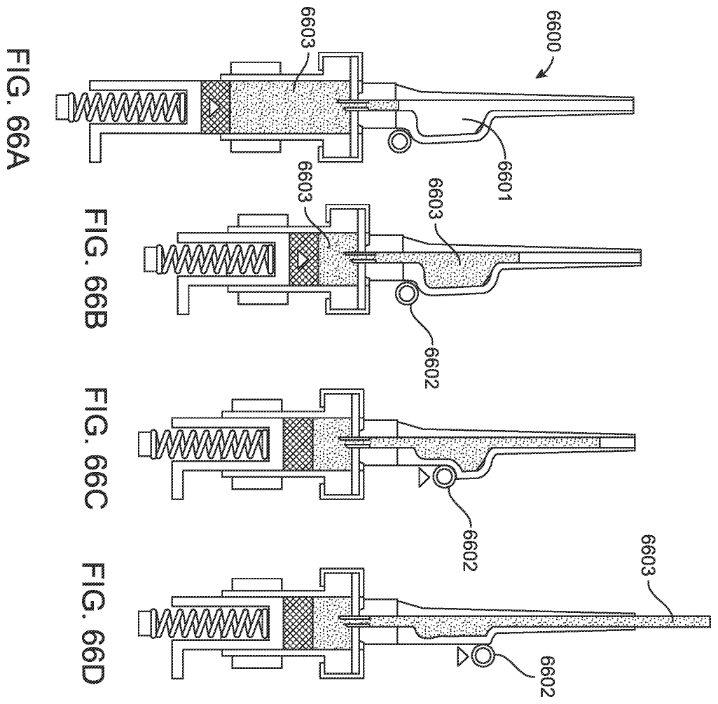

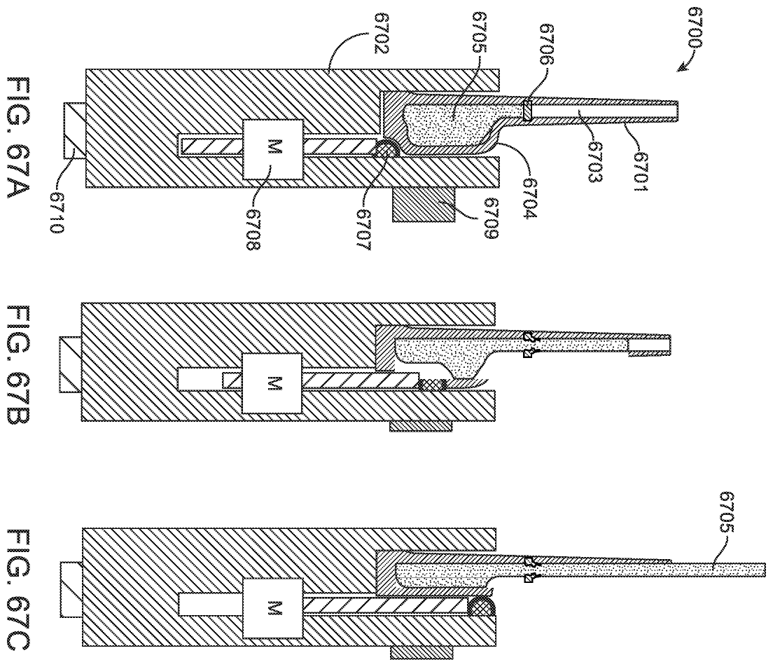

FIGS. 65A-C shows an exemplary intranasal drug delivery device 6500 according to some embodiments. In some embodiments, a drug container 6508 containing a drug 6505 is a ridged container toped with a septum (e.g. an ampule). In some embodiments, upon activation of the device 6500, two needles are inserted into the drug container 1) a first needle 6501 in fluidic communication with a dispensing tip 6506 and 2) a second needle 6502 in fluidic communication with a secondary chamber 6507 containing a secondary fluid 6503 (e.g., air) (FIG. 65B). In some embodiments, a pump pushes the secondary fluid 6503 into the drug container 6508, and this forces the drug 6505 out of the dispensing tip 6506 (FIG. 65C). The flow rate of the drug 6505 exiting the device 6500 is controlled by controlling the flow rate of secondary fluid 6503 into the drug container. In some embodiments, the secondary fluid (e.g., air) is moved by a spring driven piston pump 6509 and the flow rate of the secondary fluid 6503 is controlled by a flow restriction 6504 in the air path to the drug container.

FIGS. 66A-D shows an exemplary intranasal drug delivery device 6600 according to some embodiments. In some embodiments, a drug 6603 is dispensed from a drug container using a pump to propel the drug 6603 out of the device 6600. ((FIGS. 66C-D). In this embodiment, the pump is a peristaltic pump integrated into the dispensing tip body 6601 (FIGS. 66A-B), but the pump may be a piston pump, peristaltic pump, gear pump, bellows, elastic chamber, or similar device. The flow rate for ejecting the drug 6603 is controlled by or determined by the operation of the pump. Here flow rate of the drug 6603 exiting the device 6600 is determined by the rate of travel of roller 6602. In some embodiments, the roller 6602 is driven by a spring combined with a dashpot damper to provide a controlled flow rate. The pump may also be driven by electronic drive system.

FIG. 67A shows an exemplary intranasal drug delivery device 6700 consisting of a disposable element 6701 and a reusable dispensing tool 6702. In some embodiments, the disposable element 6701 contains a drug 6705 for storage and provides clean and sterile surfaces to contact the patient. In some embodiments, only the disposable element 6701 comes in contact with the drug 6705. In some embodiments, the disposable element 6701 may come in a sterile package ready for use. The disposable element 6701 may provide a surface that covers the end of the dispensing tool 6702 for contacting the exterior of the patients nose and the patients face, or portions of the dispensing tool 6702 may be wiped down or otherwise cleaned/sterilized (e.g. UV sterilization) to ensure safe contact with the patient and safe use between patients. In some embodiments, the disposable element 6701 contains a dispensing tip 6703, drug formulation container 6704, a volume of drug 6705, and a burst membrane 6706. The reusable dispensing tool 6702 contains an interface to connect to the disposable element 6701 (e.g. a taper fit, a socket, a snap fit, a threaded stud), a moving element 6707 to press on the disposable element 6701, a drive system 6708 for the moving element, and a trigger 6709. In this embodiment, the disposable element 6701 is contained in a socket, the moving element 6707 is a roller, the drug container 6704 is a flexible bag, and the drive system 6708 is an electric motor. The moving element may also be a plunger, a cam, or a bag/bellows inflated by the drive system. The drug container may be any standard drug container with at least one movable surface (e.g. cartridge, blister pack, blow mold fill container), or it may be a custom molded chamber with at least one movable or flexible surface (e.g. a ridged molded cartridge capped with a flexible membrane). The burst membrane may be a plastic membrane, a one-way valve, a ridged cap that disconnects with a snap fit, a pressure relief valve, or any other mechanism that allows fluid to flow only after a prescribed minimum pressure is applied. The drive system may consist of a spring, pressurized gas, or vacuum chamber. The drive system may be energized by force applied by the user in an activation step. In some embodiments, to use the device, a user attaches the disposable element 6701 to the dispensing tool 6702 and inserts the dispensing tip 6703 into their nose. In some embodiments, when the user presses trigger 6709, the moving element 6707 presses on the drug container 6704 (FIG. 67B). This pressurizes the drug container body and causes the burst membrane 6706 to break (FIG. 67B). In some embodiments, the moving element 6707 then continues to move at a controlled velocity (FIG. 67C), which dispenses the drug 6705 out of the device 6700 with a controlled flow rate into the nose. Velocity control for the moving element may be provided by feedback or control, open loop control if the drive element is electric. Velocity control may also be provided by a viscous damping element if the system is driven mechanically. The moving element may pause momentarily after the burst membrane breaks. The reusable tool may include an eject button 6710 to easily remove the disposable element 6701. The eject button may both eject the disposable element 6701 and energize the drive system with one press. Both the trigger and the eject button may be a button, a slider, or a switch.

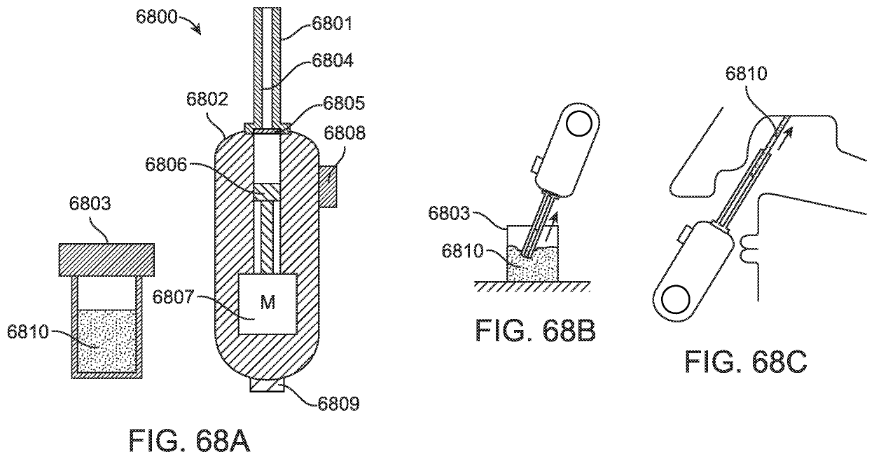

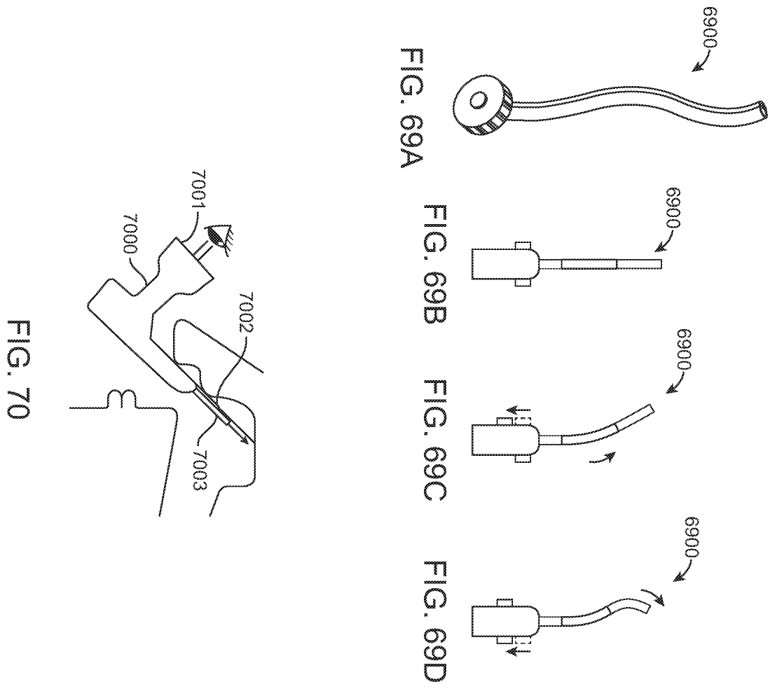

FIG. 68A shows an exemplary intranasal drug delivery device 6800 comprising a disposable element 6801, a reusable dispensing tool 6802, and a separate drug container 6803. In some embodiments, the disposable element 6801 comprises a dispensing tip 6804 and may contain a membrane or similar barrier 6805 to a) prevent fluid moving into the reusable dispensing tool 6802 and/or b) prevent contaminants (e.g. dust, bacteria, oils, etc.) entering the disposable element 6801. In some embodiments, the disposable element 6801 may have an open construction without a barrier. The disposable element 6801 may be a single molded piece of plastic or elastomer. Here the disposable element 6801 can provide clean and sterile surfaces to contact the patient. In some embodiments, only the disposable element 6801 comes in contact with the drug. The disposable element may come in a sterile package ready for use. The disposable element 6801 may provide a surface that covers the end of the dispensing tool 6802 for contacting the exterior of the patients nose and the patients face, or portions of the dispensing tool 6802 may be wiped down or otherwise cleaned/sterilized (e.g. UV sterilization) to ensure safe contact with the patient and safe use between patients. In this embodiment, the drug container 6803 is a screw top vial. In some embodiments, the drug container 6803 may be a blow mold fill ampule, blister pack, or any other standard drug container. In some embodiments, the reusable dispensing tool 6802 comprises a means to draw a vacuum and/or to pump air into the disposable element. In this embodiment, vacuum and air pressure is generated using a piston 6806, a drive element 6807, and a trigger 6808. The drive element 6807 may be an electric motor, solenoid, voice, coil, spring/damper system, spring, or other similar mechanism. In some embodiments, to use the device 6800, the disposable element 6801 is attached to the reusable tool 6802 (FIG. 68A). In this embodiment, the disposable element 6801 force fits onto the reusable tool 6802 with a taper fit. The disposable element 6801 may also be coupled to the reusable tool 6802 using a screw, socket, ball detent, or a standard pneumatic or hydraulic interface. In some embodiments, the drug container 6803 is opened and the disposable element is inserted into the volume of a drug 6810 (FIG. 68B). In some embodiments, once the trigger 6808 is pressed, the piston 6806 draws a vacuum. In some embodiments, the vacuum draws a measured dose of the drug 6810 into the dispensing tip 6804 of the disposable element (FIG. 68B). In some embodiments, the device is then removed from the drug container 6803 and inserted into a patient's nasal cavity. In some embodiments, the trigger is pressed a second time (or a secondary trigger is pressed), and then the device pumps air into the dispensing tip 6804 of the disposable element 6801 with a controlled flow rate. In some embodiments, the air pushes the drug 6810 out of the disposable element and into the patient's nasal cavity (FIG. 68C) at a controlled flow rate. The reusable tool 6802 may also contain an eject button 6809 to quickly remove the disposable element 6801. The eject button 6809 may both eject the disposable element 6801 and energize the drive system 6807 with one press. Both the trigger and the eject button may be a button, a slider, a switch, or other standard user interface. This device may be used without a drug formulation container 6803. In one embodiment, the device is used without a drug formulation container in a setting where the drug (e.g. stem cells) is prepared on site, transferred to an open container, loaded into the device as shown in FIG. 68B, and given immediately to the patient.

FIG. 69A shows an exemplary dispensing tip 6900 for an intranasal drug delivery device 3900 described herein. In some embodiments, the dispensing tip 6900 is integrated into an endoscope so the user can control the shape of the dispensing tip as it is inserted (FIG. 69B-D). In some embodiments, this allows a user to compensate for anatomical variation between patients and accurately aim the dispensing tip at a target site within a patient's nasal cavity. The dispensing tip may also include a fiber optic cable to visualize the target site.

FIG. 70 shows an accessory that could be integrated into an intranasal drug formulation delivery device 4000 described herein. In some embodiments, the accessory comprises an eye piece 7001, internal optics, an internal light source, and a narrow viewing element (e.g. a fiber optic cable) 7002. In some embodiments, the accessory is integrated into an intranasal delivery device described herein so that the viewing element is aligned with the corresponding device dispensing tip 7003. In some embodiments, the internal light source illuminates where the dispensing tip 7003 is pointing, and a user is able to visualize where the dispensing tip 7003 is pointing to help guide device insertion into a patient's nasal cavity. In some embodiments, this also allows the user to confirm that drug delivery was successful. The system may also use a digital camera rather than an eye piece with optics, and the operator will visualize the target site of a patient's nasal cavity on a screen.

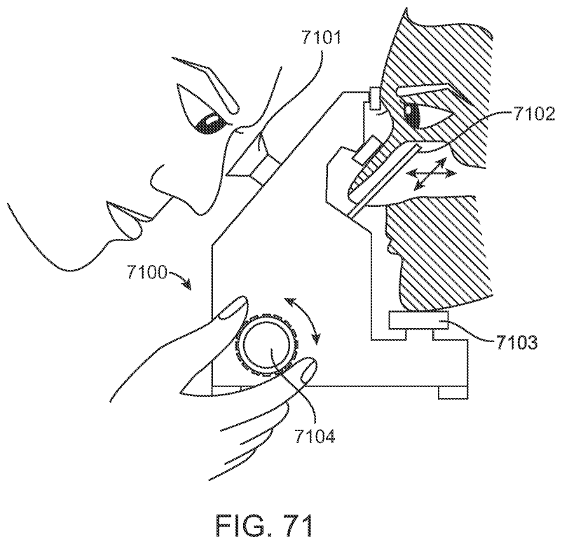

FIG. 71 shows an exemplary intranasal drug delivery device 7100. In some embodiments, the device 7100 comprises an eye piece 7101, a dispensing tip 7102, a patient positioning rest 7103, a disposable dispensing tip, a disposable drug cartridge, and positioning controls 7104. In some embodiments, the disposable dispensing tip and disposable drug cartridge are loaded into the device. In some embodiments, the drug cartridge contains a drug to be dispensed into a patient's nasal cavity. In some embodiments, a patient then rests their head on the positioning rest 7103, and an operator guides the dispensing tip 7102 into the patient's nasal cavity while visualizing the procedure through an eye piece. In some embodiments, the operator can then dispense a drug out of the drug cartridge (located in the device 7100), through the dispensing tip 7102, and into a target site of the patient's nasal cavity. In some embodiments, the operator can visualize through the eye piece that the drug delivery to the target site was successful. In some embodiments, the system may also use a digital camera rather than an eye piece with optics, and the operator will visualize the target site on a screen.

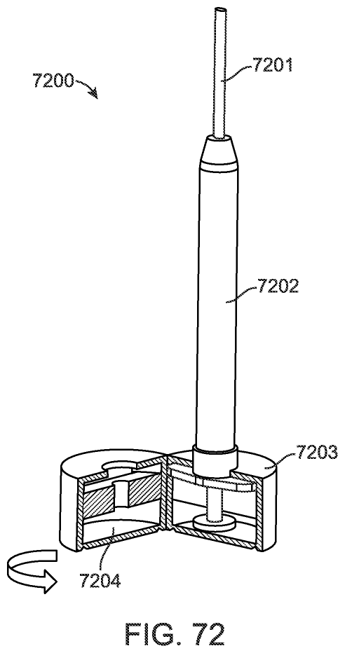

FIG. 72 shows an exemplary intranasal drug deliver device 7200 according to some embodiments. In some embodiments, the device comprises a dispensing tip 7201 a syringe 7202, and a cap 7303. In some embodiments, the cap 7303 is a single injection molded part that attaches to the syringe 7202 with a snap fit. In some embodiments, the cap 7203 includes a shearing element (e.g. shear pin, shear plane, stress concentration, snap lock) 7204. In some embodiments, a user presses on the shearing element 7204 until it breaks. In some embodiments, breaking the shearing element 7204 delivers a controlled force to the syringe plunger and a drug is forced out of the syringe 7202 and out of the dispensing tip 7201. In some embodiments, the dispensing tip 7201 includes a flow restriction (e.g. orifice plate, narrow channel, constant flow rate valve) that controls the drug dispensing rate and provides an appropriate velocity profile to eject the drug to a target site within a patient's nasal cavity. The shearing element 7204 may also be an overcenter mechanism, ball detent, or other mechanical element that releases under a specific force.

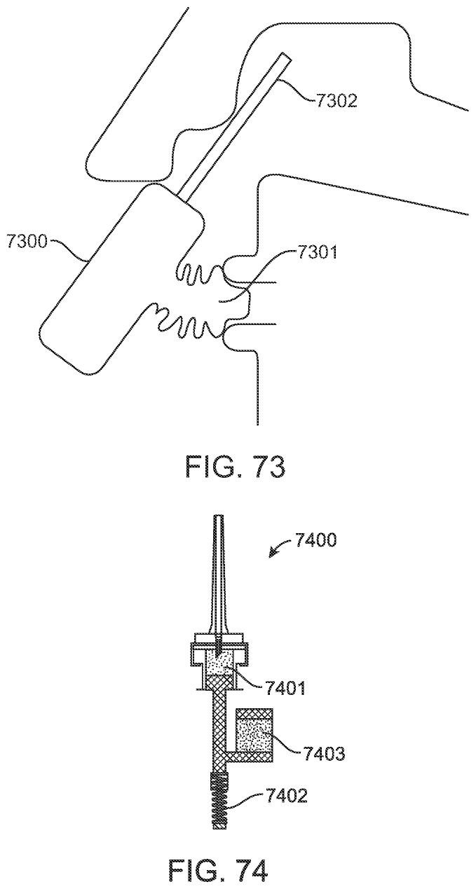

FIG. 73 shows an exemplary intranasal drug delivery device 7300 according to some embodiments. In some embodiments, the device 7300 comprises a mouth piece 7301, and dispensing tip 7302. In some embodiments, a user inserts the dispensing tip 7302 into the nose and the mouth piece 7301 mouth as shown in FIG. 73. In some embodiments, the user then applies positive pressure to the device 7300 by blowing into the mouth piece 7301. In some embodiments, the flow of air into the mouth piece 7301 dispenses a drug through the dispensing tip 7302 into a target site of a user's nasal cavity. In some embodiments, the drug may be held in a flexible container (e.g. bag, blister pack, bellows) that is directly compressed by air flow from the user. The drug may be driven by a mechanism that is driven by the air flow from the user (e.g. air from the user drives a large diaphragm that moves a plunger of a drug cartridge). In some embodiments, flow rate of the drug may be regulated by a flow restriction (e.g. orifice plate, narrow channel, constant flow rate valve) in the path of air from the user or in the path of drug out of the dispensing tip 7302. The air flow may fill a compliant chamber where pressure in the chamber builds to a specific pressure regulated by a valve (burst valve, pressure relieve valve, etc) before pushing the drug out of the device. The device may also include a priming step to move the drug out of the drug container (e.g. cartridge, ampule) and into a secondary chamber. In some embodiments, air flow into the secondary chamber directly contacts the drug and forces it through the dispensing tip 7302. This priming step may be driven by air pressure from the user, so the sequence of priming then ejection occurs with a single user breath.

### Exemplary Embodiments

In accordance with an aspect, there is provided an intranasal drug delivery device having compliant or flexible, soft nib to precisely locate the dosage and provide comfort for user. The term drug can also be used herein to refer to other agents such as vitamins, fragrance, saline or non-pharmaceutical agents.

In accordance with an aspect, there is provided an intranasal drug delivery device having a cocking mechanism and actuator to load and release dosage.

In accordance with an aspect, there is provided an intranasal drug delivery device having a non-air interface mechanically pressurized fluid reservoir to enable dosing independent of orientation and to load shot chamber. In some example embodiments, reservoir can be collapsible from external pressure, including ambient air pressure.

In accordance with an aspect, there is provided an intranasal drug delivery device connectable to a facial or device recognition application to prevent intentional or unintentional misuse.

In accordance with an aspect, there is provided an intranasal fluid delivery device comprising a dispensing tip connected to a hollow needle, a shot chamber carrying a fluid, the shot chamber having a diaphragm at one end and a plunger at the other end, and an actuator connected to a push rod moveable toward the shot chamber and having a locking mechanism, wherein pushing the actuator releases the locking mechanism, allowing the push rod to push against the plunger, exerting pressure on the fluid and forces the needle through the diaphragm into the shot chamber such that the fluid flows out of the needle into the dispensing tip.

In accordance with an aspect, there is provided apparatus for delivering fluid to a nasal volume comprising a housing having a first end with a dispensing opening and a second end with an actuating opening, a dispensing tip coupled to the dispensing opening, a capsule within the housing between the actuating opening and the dispensing opening, the capsule comprising a tube pre-filled with fluid between a diaphragm and a plunger, and, an actuator coupled to the actuating opening, the actuator comprising a push rod moveable into contact with the plunger and held back by a locking mechanism, and a spring urging the push rod toward the plunger.

In accordance with an aspect, there is provided a method for targeted intranasal fluid delivery. The method comprises inserting a compliant dispensing tip into a nasal cavity, and ejecting a fluid from the compliant dispensing tip to deliver a liquid jet to a targeted region within the nasal cavity. The targeted region may be an olfactory region of the nasal cavity. Inserting the compliant dispensing tip into the nasal cavity may comprises inserting the compliant dispensing tip at least into an upper nares. Inserting the compliant dispensing tip into the nasal cavity may comprise positioning an end of the compliant dispensing tip proximate to the olfactory region or the anterior entry to the olfactory region. The compliant dispensing tip may comprise a cannula. Ejecting the fluid may comprise ejecting the fluid with a controlled velocity profile to limit shear forces on the fluid.

In some embodiments, the actuator is connected to a spring and the spring is connected to the push rod. In some embodiments, the locking mechanism comprises one or more tabs made from a lock material, and the locking mechanism is released by breaking the lock material. In some embodiments, the locking mechanism comprises one or more pivotable tabs, and the locking mechanism is released by pivoting the tabs. In some embodiments, the device comprises an outer chassis having a pair of foldable arms on opposed sides thereof. In some embodiments, the dispensing tip comprises a cannula. In some embodiments, the dispensing tip comprises an atomizer. In some embodiments, pushing the actuator forces the needle through the diaphragm into the shot chamber and then releases the locking mechanism allowing the push rod to push against the plunger. In some embodiments, the intranasal fluid delivery device further comprises a damping mechanism to create a controlled velocity profile of the fluid when it exits the dispensing tip. In some embodiments, the damping mechanism comprises at least one of a magnet, a spring, a viscous damper, a sealed chamber with an airflow restriction, a container of compressed gas, a valve, a motor, an elastomeric chamber, a flow restriction device, and a configuration of the plunger and shot chamber. In some embodiments, intranasal fluid delivery device comprises compliant or flexible, soft nib designed to conform to aspects of the nasal cavity anatomy such as the nasal valve, nasal turbinates and the septum and to precisely locate the dosage and provide comfort for user. In some embodiments, the dispensing tip is flexible In some embodiments, the dispensing tip has an elliptical cross section. In some embodiments, the dispensing tip has a distal portion and a proximal portion, and wherein a first center axis of the distal portion and a second center axis of the proximal portion are non-colinear. In some embodiments, the dispensing tip has a distal portion having a first rigidity and a proximal portion having a second rigidity, and wherein the first rigidity is less than the second rigidity. In some embodiments, the dispensing tip has a distal portion having a first softness and a proximal portion having a second softness, and wherein the first softness is less than the second softness. In some embodiments, the dispensing tip comprises an off-center drug dispensing channel distal to the distal portion. In some embodiments, the dispensing tip comprises a protruding element. In some embodiments, the dispensing tip comprises an inflatable balloon surrounding at least a part of the distal portion of the dispensing tip. In some embodiments, the inflatable balloon further surrounds at least a part of the proximal portion of the dispensing tip. In some embodiments, a distal portion of the dispensing tip is curved. In some embodiments, the dispensing tip has a distal portion having a first outer diameter and a proximal portion having a second outer diameter, and wherein the first outer diameter is less than the second outer diameter. In some embodiments, the dispensing tip has a distal portion having a first outer diameter, a proximal portion having a second outer diameter, and wherein the first outer diameter is greater than the second outer diameter. In some embodiments, the dispensing tip has a proximal portion having an outer diameter that tapers towards the distal portion to provide comfort for the user or to limit insertion distance. In some embodiments, the dispensing tip has a perforation. In some embodiments, the perforation is on a distal portion of the dispensing tip. In some embodiments, the perforation is on a single side of the dispensing tip. In some embodiments, a distal portion of the dispensing tip has a spiral shape. In some embodiments, exerting the pressure on the fluid increases a radius of the spiral shape.

In accordance with an aspect, there is provided apparatus for delivering fluid to a nasal volume comprising a housing having a first end with a dispensing opening and a second end with an actuating opening, a dispensing tip coupled to the dispensing opening, a capsule within the housing between the actuating opening and the dispensing opening, the capsule comprising a tube pre-filled with fluid between a diaphragm and a plunger, and, an actuator coupled to the actuating opening, the actuator comprising a push rod moveable into contact with the plunger and held back by a locking mechanism, and a spring urging the push rod toward the plunger.

In some embodiments, the apparatus further comprises means for damping a flow of fluid ejected from the dispensing tip. In some embodiments, the locking mechanism comprises pivotable tabs. In some embodiments, the locking mechanism comprises breakable tabs. In some embodiments, the dispensing tip is flexible. In some embodiments, the dispensing tip has an elliptical cross section. In some embodiments, the dispensing tip has a distal portion and a proximal portion, and wherein a first center axis of the distal portion and a second center axis of the proximal portion are non-colinear. In some embodiments, the dispensing tip has a distal portion having a first rigidity and a proximal portion having a second rigidity, and wherein the first rigidity is less than the second rigidity. In some embodiments, the dispensing tip comprises an off-center drug dispensing channel distal to the distal portion. In some embodiments, the dispensing tip comprises a protruding element. In some embodiments, the dispensing tip comprises an inflatable balloon surrounding at least a part of the distal portion of the dispensing tip. In some embodiments, the inflatable balloon further surrounds at least a part of the proximal portion of the dispensing tip. In some embodiments, a distal portion of the dispensing tip is curved In some embodiments, the dispensing tip has a distal portion having a first outer diameter and a proximal portion having a second outer diameter, and wherein the first outer diameter is less than the second outer diameter. In some embodiments, the dispensing tip has a distal portion having a first outer diameter, a proximal portion having a second outer diameter, and wherein the first outer diameter is less than the second outer diameter. In some embodiments, the dispensing tip has a perforation. In some embodiments, the perforation is on a distal portion of the dispensing tip. In some embodiments, the perforation is on a single side of the dispensing tip. In some embodiments, a distal portion of the dispensing tip has a spiral shape. In some embodiments, exerting the pressure on the fluid increases a radius of the spiral shape.

In accordance with an aspect, there is provided a method for targeted intranasal fluid delivery. The method comprises inserting a compliant dispensing tip into a nasal cavity and ejecting a fluid from the compliant dispensing tip to deliver a liquid jet or stream to a targeted region within the nasal cavity. The targeted region may be an olfactory region of the nasal cavity. Inserting the compliant dispensing tip into the nasal cavity may comprises inserting the compliant dispensing tip at least into an upper nares. Inserting the compliant dispensing tip into the nasal cavity may comprise positioning an end of the compliant dispensing tip proximate to the olfactory region or to the anterior entry to the olfactory region. The compliant dispensing tip may comprise a cannula. Ejecting the fluid may comprise ejecting the fluid with a controlled velocity profile to limit shear forces on the fluid.

In various further aspects, the disclosure provides corresponding systems and devices, and logic structures such as machine-executable coded instruction sets for implementing such systems, devices, and methods.

In one aspect, provided herein, is a dispensing tip for an apparatus for delivering fluid to a nasal volume comprising a hydrophilic coating applied to an outer surface of the dispensing tip, wherein the hydrophilic coating is activated by contact with a hydrating medium. In some embodiments, the hydrating medium is water, a gel, a viscous liquid, a vapor, or any combination thereof. In some embodiments, the water is water vapor. In some embodiments, activating the hydrophilic coating reduces a surface friction of the hydrophilic coating.

In one aspect, provided herein, is a packaging system for a dispensing tip comprising: a. a sealed packaging material; and b. a dispensing tip provided herein contained in a first compartment of the packaging material. In some embodiments, the hydrating medium is contained inside the first compartment. In some embodiments, the hydrating medium is contained in a second compartment of the packaging material. In some embodiments, the packaging system further comprises a membrane between the first compartment and the second compartment. In some embodiments, piercing the membrane forms a fluid connection between the first compartment and the second compartment. In some embodiments, the packaging system further comprises a one-way valve, a clamp, or both between the first compartment and the second compartment. In some embodiments, the hydrating medium is a vapor released by a hydrating medium body. In some embodiments, the hydrating medium body comprises a foam material, a fabric material, a porous plastic material, a porous ceramic material, a wicking material, or any combination thereof. In some embodiments, the hydrating medium body is encapsulated by a vapor permeable membrane. In some embodiments, the first compartment and the second compartment in fluidically connect through a tortuous chamber.

In one aspect, provided herein, is a kit comprising a. a dispensing tip provided herein and b. a hydrating medium provided herein disposed within a packaging material. In some embodiments, the packaging material is an ampule, a pouch, a square treat open pouch, or any combination thereof. In some embodiments, the hydrating medium is a gel. In some embodiments, the packaging material delivers the hydrating medium the outer surface of the dispensing tip. In some embodiments, the packaging material is configured to allow insertion of the dispensing tip into the hydrating medium.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

### Dispensing Tip with Hydrophilic Coating, Packaging System, and Kit Embodiments

Disclosed herein, in some embodiments, is a dispensing tip for an apparatus for delivering fluid to a nasal volume comprising a hydrophilic coating applied to an outer surface of the dispensing tip, wherein the hydrophilic coating is activated by contact with a hydrating medium. In some embodiments, the hydrating medium is water, a gel, a lubricating gel, a viscous liquid, a vapor, or any combination thereof. In some embodiments, the water is water vapor. In some embodiments, activating the hydrophilic coating reduces a surface friction of the hydrophilic coating. In some embodiments, the dispensing tip comprises a compliant nib, configured to comply with an intranasal passage of a subject.

Disclosed herein, in some embodiments, is a packaging system for a dispensing tip comprising: a. a sealed packaging material; and b. the dispensing tip of any embodiment herein wherein the dispensing tip comprises a hydrophilic coating applied to an outer surface of the dispensing tip, wherein the dispensing tip is contained in a first compartment of the packaging material. In some embodiments, the hydrating medium is contained inside the first compartment. In some embodiments, the hydrating medium is contained in a second compartment of the packaging material. In some embodiments, the packaging system further comprising a membrane between the first compartment and the second compartment. In some embodiments, wherein piercing the membrane forms a fluid connection between the first compartment and the second compartment. In some embodiments, the packaging system further comprising a one-way valve, a clamp, or both between the first compartment and the second compartment. In some embodiments, the hydrating medium is a vapor released by a hydrating medium body. In some embodiments, the hydrating medium body comprises a foam material, a fabric material, a porous plastic material, a porous ceramic material, a wicking material, or any combination thereof. In some embodiments, the hydrating medium body is encapsulated by a vapor permeable membrane. In some embodiments, the first compartment and the second compartment in fluidically connect through a tortuous chamber. In some embodiments, the dispensing tip comprises a compliant nib, configured to comply with an intranasal passage of a subject.

Disclosed herein, in some embodiments, is a kit comprising: a. the dispensing tip of any embodiment herein wherein the dispensing tip comprises a hydrophilic coating applied to an outer surface of the dispensing tip; and b. any the hydrating medium described herein disposed within a packaging material. In some embodiments, the packaging material is an ampule, a pouch, a square treat open pouch, or any combination thereof. In some embodiments, the hydrating medium is a gel. In some embodiments, the packaging material delivers the hydrating medium to the outer surface of the dispensing tip. In some embodiments, the packaging material is configured to allow insertion of the dispensing tip into the hydrating medium. In some embodiments, the dispensing tip is disposed within the packaging material such that the hydrophilic coating is in contact with the hydrating medium. In some embodiments, the dispensing tip comprises a compliant nib, configured to comply with an intranasal passage of a subject.

Disclosed herein, in some embodiments, is a method of using a kit disclosed herein, the method comprising: dispensing the hydrating medium onto the hydrophilic coating of the dispensing tip. In some embodiments, the dispensing tip comprises a compliant nib, configured to comply with an intranasal passage of a subject.

Disclosed herein, in some embodiments, is a method of using a kit disclosed herein, the method comprising: inserting the dispensing tip into hydrating medium disposed within the packaging material. In some embodiments, the dispensing tip comprises a compliant nib, configured to comply with an intranasal passage of a subject.

Disclosed herein, in some embodiments, is a method of using a packaging system disclosed herein, the method comprising opening the sealed packaging material; and adding the hydrating medium to the first compartment such that the hydrophilic coating is contacted with the hydrating medium. In some embodiments, the dispensing tip comprises a compliant nib, configured to comply with an intranasal passage of a subject.

Disclosed herein, in some embodiments, is a method of using a packaging system disclosed herein, the method comprising: transferring the hydrating medium from the second compartment to the first compartment such that the hydrophilic coating is contacted with the hydrating medium. In some embodiments, the dispensing tip comprises a compliant nib, configured to comply with an intranasal passage of a subject.

Disclosed herein, in some embodiments, is a packaging system for a dispensing tip comprising: a. a sealed packaging material; and b. the dispensing tip disposed in a first compartment of the packaging material. In some embodiments, the packaging system further comprising a lubricating gel. In some embodiments, the lubricating gel is disposed within the first compartment. In some embodiments, the lubricating gel is contained in a second compartment of the packaging material. In some embodiments, the packaging system further comprising a membrane between the first compartment and the second compartment. In some embodiments, wherein piercing the membrane forms a fluid connection between the first compartment and the second compartment. In some embodiments, the packaging system further comprising a one-way valve, a clamp, or both between the first compartment and the second compartment. In some embodiments, the dispensing tip comprises a compliant nib, configured to comply with an intranasal passage of a subject.

Disclosed herein, in some embodiments, is a method of lubricating a dispensing tip, comprising adding a lubricating gel to the surface of the dispensing tip. In some embodiments, the lubricating gel is dispensed from a packaging material containing the lubricating gel. In some embodiments, the dispensing tip is dipped into a packaging material containing a lubricating gel. In some embodiments, the dispensing tip comprises a compliant nib, configured to comply with an intranasal passage of a subject.

### Alternative Damping Mechanisms Embodiment

Disclosed herein, in some embodiments, is an apparatus for delivering fluid to a nasal volume, the apparatus comprising: a housing having a first end with a dispensing opening and a second end with an actuating opening; a dispensing tip coupled to the dispensing opening; a capsule within the housing between the actuating opening and the dispensing opening, the capsule comprising a tube pre-filled with fluid between a diaphragm and a plunger; and an actuator coupled to the actuating opening, the actuator comprising a push rod moveable into contact with the plunger and held back by a locking mechanism, and a spring urging the push rod toward the plunger. In some embodiments, the apparatus further comprising means for damping a flow of fluid ejected from the dispensing tip. In some embodiments, the means for damping a flow of fluid comprises a flow restriction fluidically coupling the needle and the dispensing tip. In some embodiments, the means for damping a flow of fluid comprises a flow restriction fluidically coupling the needle and the dispensing tip. In some embodiments, the flow restriction is a constriction within the hollow needle. In some embodiments, the flow restriction is a porous body. In some embodiments, the porous body comprises an open cell pore, a closed cell pore, or any combination thereof. In some embodiments, the porous body is formed of metal, ceramic, plastic, wood, or any combination thereof. In some embodiments, the flow restriction is an orifice plate within the needle, the dispensing tip, or both. In some embodiments, the flow restriction is an orifice within the needle, the dispensing tip, or both. In some embodiments, the flow restriction comprises flexible washers within the needle, the dispensing tip, or both. In some embodiments, the damping mechanism comprises an actuator restriction coupled to the actuator. In some embodiments, the actuator restriction comprises a porous cavity. In some embodiments, the porous cavity comprises an open cell pore, a closed cell pore, or any combination thereof. In some embodiments, the porous cavity is formed of metal, ceramic, plastic, wood, or any combination thereof. In some embodiments, the dispensing tip comprises a compliant nib, configured to comply with an intranasal passage of a subject.

### Secondary Chamber Embodiment

Disclosed herein, in some embodiments, is an intranasal fluid delivery device comprising a dispensing tip connected to a hollow needle, a shot chamber carrying a fluid, the shot chamber having a diaphragm at one end and a plunger at the other end, and an actuator connected to a push rod moveable toward the shot chamber and having a locking mechanism, wherein pushing the actuator releases the locking mechanism, allowing the push rod to push against the plunger, exerting pressure on the fluid and forcing the needle through the diaphragm into the shot chamber such that the fluid flows out of the needle into the dispensing tip and a secondary chamber, wherein the secondary chamber comprises a second plunger at one end and a second actuator connected to a second push rod moveable toward the dispensing tip. In some embodiments, the needle comprises a one-way valve configured to prevent backflow. In some embodiments, the second plunger and the second actuator are configured to drive the fluid out of the dispensing tip. In some embodiments, the second plunger and the second actuator are further configured to drive a secondary fluid out of the dispensing tip. In some embodiments, the secondary fluid is a gas. In some embodiments, the intranasal fluid delivery device further comprising a second needle for providing fluid communication between the shot chamber and the secondary chamber. In some embodiments, the second actuator is configured to control the flow rate of the fluid out of the dispensing tip. In some embodiments, the dispensing tip comprises a compliant nib, configured to comply with an intranasal passage of a subject.

Disclosed herein, in some embodiments, is an apparatus for delivering fluid to a nasal volume, the apparatus comprising: a housing having a first end with a dispensing opening and a second end with an actuating opening; a dispensing tip coupled to the dispensing opening, the dispensing tip comprising a pump configured to propel fluid out of the dispensing tip; a capsule within the housing between the actuating opening and the dispensing opening, the capsule comprising a tube pre-filled with fluid between a diaphragm and a plunger; and, an actuator coupled to the actuating opening, the actuator comprising a push rod moveable into contact with the plunger and held back by a locking mechanism, and a spring urging the push rod toward the plunger. In some embodiments, the pump comprises a roller configured to control the flow rate of fluid out of the dispensing tip. In some embodiments, the roller is driven by a spring combined with a dashpot damper. In some embodiments, the pump is a peristaltic pump, a piston pump, a gear pump, a bellows, or an elastic chamber, or any combination thereof. In some embodiments, the pump is integrated into the dispensing tip. In some embodiments, the dispensing tip comprises a compliant nib, configured to comply with an intranasal passage of a subject.

### Burst Membrane Embodiment

Disclosed herein, in some embodiments, is an intranasal fluid delivery system comprising: a container comprising a dispensing tip and sealed by a burst membrane within the dispensing tip; a fluid within the container; and a dispensing mechanism configured to compress the container and pressurize the fluid therein to puncture the burst membrane and release the fluid from the dispensing tip. In some embodiments, at least a portion of the container is flexible. In some embodiments, the container comprises a fastener that removably couples the container to the dispensing mechanism. In some embodiments, the dispensing mechanism comprises a container release that decouples the container from the dispensing mechanism. In some embodiments, a trigger within the dispensing mechanism initiates the compression by the driving mechanism. In some embodiments, the burst membrane comprises a thin membrane, a one-way valve, a snap fit, a pressure relief valve, or any combination thereof. In some embodiments, the dispensing mechanism comprises a roller, a motor, a solenoid, a cam, a plunger, a bellow, a screw drive, a spring, a pressurized container, a vacuum chamber or any combination thereof. In some embodiments, the dispensing mechanism comprises the roller translated by a motor or a solenoid. In some embodiments, the dispensing mechanism continues to compress the chamber after the burst membrane has been punctured. In some embodiments, the dispensing mechanism comprises a velocity control controlling a velocity of the compression. In some embodiments, the velocity control comprises a damping element. In some embodiments, the velocity control pauses the compression of the container after the burst membrane is punctured. In some embodiments, the dispensing tip comprises a compliant nib, configured to comply with an intranasal passage of a subject.

### Fluid Contained in a Container Embodiment

Disclosed herein, in some embodiments, is an intranasal fluid delivery system comprising: a container containing a fluid; a hollow dispensing tip having a distal opening and a proximal opening; and a dispensing tool removably coupled to the dispensing; tip and configured to draw the fluid from the container into the proximal opening of the hollow dispensing tip, and to subsequently eject the fluid in the hollow dispensing tip intranasally. In some embodiments, a portion of the interior of the hollow dispensing tip that is proximal to the dispensing tool comprises a barrier that is air permeable and impermeable to the fluid. In some embodiments, the dispensing tool draws the fluid from the container by depressurizing a chamber within the dispensing tool and in fluid communications with the hollow dispensing tip. In some embodiments, the dispensing tool depressurizes the chamber by a motor, a solenoid, a spring, a damper, or any combination thereof. In some embodiments, the dispensing tool removably coupled to the hollow dispensing tip via a screw, a socket, a detent, a hydraulic interface, or any combination thereof. In some embodiments, at least one of the drawing of the fluid from the container and the ejecting of the fluid in the hollow dispensing tip is performed by actuating a trigger of the dispensing tool. In some embodiments, the hollow dispensing tip is removable from the dispensing tool by actuating an ejection trigger. In some embodiments, the system further comprising a sterility cover encompassing at least a portion of the dispensing tool. In some embodiments, the dispensing tip comprises a compliant nib, configured to comply with an intranasal passage of a subject.

### Dispensing Tip Coupled to an Endoscope Embodiment

Disclosed herein, in some embodiments, is an intranasal fluid delivery device comprising: an endoscope; and a dispensing tip removably coupled to the endoscope. In some embodiments, the endoscope and the dispensing tip are concentric when coupled. In some embodiments, the endoscope and the dispensing tip are tangent when coupled. In some embodiments, the endoscope and the dispensing tip are flexible. In some embodiments, the endoscope comprises an illuminator. In some embodiments, the endoscope comprises a fiber optic cable transmitting light emitted by the illuminator. In some embodiments, the endoscope further comprises a camera, an eyepiece, or both. In some embodiments, the device further comprising a patient positioning rest. In some embodiments, the device further comprising an adjustment mechanism translating the endoscope and the dispensing tip with respect to the patient positioning rest. In some embodiments, the device further comprising a sterility cover encompassing at least a portion of the patient positioning rest. In some embodiments, the device further comprising a fluid container containing a fluid and in fluidic communication with the dispensing tip. In some embodiments, the dispensing tip is decoupled from the endoscope by actuating an ejection trigger. In some embodiments, the dispensing tip comprises a compliant nib, configured to comply with an intranasal passage of a subject.

### Cap Embodiment

Disclosed herein, in some embodiments, is an apparatus for delivering fluid to a nasal volume, the apparatus comprising: a housing comprising a first end having a dispensing opening and a second end having an actuating opening; a dispensing tip coupled to the dispensing opening; a capsule within the housing between the actuating opening and the dispensing opening, the capsule comprising a diaphragm, a plunger, and a tube pre-filled with fluid between a diaphragm and a plunger; an actuator coupled to the actuating opening, the actuator comprising a push rod moveable into contact with the plunger and coupled to a locking mechanism, and a spring translating the push rod toward the plunger; and a cap comprising a shearing element covering the push rod. In some embodiments, the shearing element comprises a shear pin, a shear plane, a stress concentration, a snap lock, an overcenter mechanism, a ball detent, or combinations thereof. In some embodiments, the shearing element is configured to break when under force. In some embodiments, the shearing element is further configured to deliver a controlled force to the push rod. In some embodiments, the cap couples to the housing. In some embodiments, the cap couples to the housing by a snap fit. In some embodiments, the dispensing tip comprises a flow restriction. In some embodiments, the flow restriction is an orifice plate, a narrow channel, a constant flow rate valve, or any combination thereof. In some embodiments, the dispensing tip comprises a compliant nib, configured to comply with an intranasal passage of a subject.

### Mouthpiece Embodiment

Disclosed herein, in some embodiments, is an apparatus for delivering fluid to a nasal volume, the apparatus comprising: a housing having a dispensing opening and an actuating opening; a dispensing tip coupled to the dispensing opening; a capsule containing a first fluid and positioned within the housing between the actuating opening and the dispensing opening; and, a mouth piece in fluid connection with the actuating opening, wherein the actuating opening is configured to dispense the first fluid from the dispensing tip when a second fluid is delivered into the mouthpiece. In some embodiments, the capsule comprises a flexible capsule. In some embodiments, the flexible capsule comprises a bag, a blister pack, a bellows, or combinations thereof. In some embodiments, the flexible capsule is configured to compress when the second fluid is delivered into the mouthpiece. In some embodiments, the apparatus further comprising an actuator coupled to the actuating opening, wherein the actuator is configured to drive the first fluid out of the dispensing tip when the second fluid is delivered into the mouthpiece. In some embodiments, the actuator is a plunger. In some embodiments, the apparatus further comprising a diaphragm between the mouthpiece and the actuator. In some embodiments, the diaphragm is configured to drive the actuator when the second fluid is delivered into the mouthpiece. In some embodiments, the mouthpiece, the dispensing tip, or both comprise a flow restriction. In some embodiments, the flow restriction is an orifice plate, a narrow channel, a constant rate flow valve, or any combination thereof. In some embodiments, the apparatus further comprising a chamber between the mouthpiece and the capsule, the chamber comprising a valve configured to drive the fluid out of the dispensing tip when a certain pressure of the second fluid is reached. In some embodiments, the valve is a burst valve or a pressure release valve. In some embodiments, the apparatus further comprising a priming chamber between the capsule and the dispensing tip. In some embodiments, the priming chamber is in fluid connection with the capsule and the dispensing tip. In some embodiments, the mouthpiece is in fluid connection with the priming chamber. In some embodiments, the second fluid is air. In some embodiments, the dispensing tip comprises a compliant nib, configured to comply with an intranasal passage of a subject.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the term “generally” refers to a geometric relationship between two or more elements within tolerances of 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “subject” can refer to a “patient” or a “user”.

As used herein, the term “user” can refer to a “patient” or a “subject”.

As used herein, the term “intranasal passage” and “nasal cavity” may be used interchangeably.

The foregoing discussion provides many example embodiments of the inventive subject matter. Although each embodiment represents a single combination of inventive elements, the inventive subject matter is considered to include all possible combinations of the disclosed elements. In one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein may be implemented by physical computer hardware, including computing devices, servers, receivers, transmitters, processors, memory, displays, and networks. The embodiments described herein may comprise useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. An intranasal fluid delivery device comprising:
a. a dispensing tip comprising a flexible nib configured to comply with or conform to a surface of a subject's nasal cavity, thereby enabling a fluid delivery orifice of the dispensing tip to be directed towards an olfactory region of the subject;
b. a shot chamber for carrying a fluid, the shot chamber fluidly coupled to the dispensing tip; and
c. a plunger configured to drive the fluid from the shot chamber and through the dispensing tip, thereby delivering the fluid from the fluid delivery orifice of the dispensing tip.

2. An intranasal fluid delivery device comprising:
a dispensing tip comprising a flexible nib configured to comply with or conform to a surface of a subject's nasal cavity, thereby enabling a fluid delivery orifice of the dispensing tip to be directed towards an olfactory region of the subject for delivering a fluid therein.

3. The intranasal fluid delivery device of claim 1 or 2, wherein the dispensing tip is configured to deliver the fluid as a liquid jet or a liquid stream.

4. The intranasal fluid delivery device of claim 3, wherein the liquid jet is a laminar flow or the liquid stream is a laminar flow.

5. The intranasal fluid delivery device of claim 1 or 2, wherein the dispensing tip is configured to deliver the fluid as a spray, mist, or aerosol.

6. The intranasal fluid delivery device of claim 1 or 2, wherein the fluid comprises a powder.

7. The intranasal fluid delivery device of claim 1 or 2, wherein the dispensing tip comprises an atomizer.

8. The intranasal fluid delivery device of claim 1 or 2, wherein the dispensing tip comprises a cannula.

9. The intranasal fluid delivery device of claim 1 or 2, wherein the dispensing tip is tubular.

10. The intranasal fluid delivery device of claim 9, wherein the dispensing tip has an inner diameter from about 0.3 mm to about 1.5 mm.

11. The intranasal fluid delivery device of claim 1 or 2, wherein the flexible nib comprises a polymer.

12. The intranasal fluid delivery device of claim 1 or 2, wherein the flexible nib comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof.

13. The intranasal fluid delivery device of claim 1 or 2, wherein the dispensing tip comprises a distal portion having a first rigidity and a proximal portion having a second rigidity, and wherein the first rigidity is less than the second rigidity.

14. The intranasal fluid delivery device of claim 13, wherein the distal portion comprising a first rigidity comprises a portion of the dispensing tip that is about 1 mm to about 15 mm from the fluid delivery orifice.

15. The intranasal fluid delivery device of claim 1 or 2, wherein the dispensing tip comprises a distal portion that is softer than a proximal portion.

16. The intranasal fluid delivery device of claim 15, wherein the distal portion that is softer than a proximal portion comprises a portion of the dispensing tip that is about 1 mm to about 15 mm from the fluid delivery orifice.

17. The intranasal fluid delivery device of claim 1 or 2, wherein the dispensing tip comprises a distal portion having a first outer diameter and a proximal portion having a second outer diameter, and wherein the first outer diameter is less than the second outer diameter.

18. The intranasal fluid delivery device of claim 1 or 2, wherein the dispensing tip further comprises a nose cushion to limit over-insertion of the dispensing tip within the nasal cavity, wherein the nose cushion configured to provide subject comfort.

19. The intranasal fluid delivery device of claim 1 or 2, wherein the dispensing tip comprises a distal portion having a first outer diameter and a proximal portion having a second outer diameter, and wherein the first outer diameter is greater than the second outer diameter.

20. The intranasal fluid delivery device of claim 1 or 2, wherein the dispensing tip is configured to be inserted to an insertion depth of about 10 mm to about 90 mm within the subject's nasal cavity.

21. The intranasal fluid delivery device of claim 1 or 2, wherein the dispensing tip is configured to be inserted to an insertion depth of about 40 mm to about 70 mm within the subject's nasal cavity.

22. The intranasal fluid delivery device of claim 1 or 2, wherein the fluid delivery orifice of the dispensing tip is configured to be positioned at or near the anterior entry to the olfactory region.

23. The intranasal fluid delivery device of claim 1 or 2, wherein the fluid delivery orifice of the dispensing tip is configured to be positioned in the upper nares of a subject.

24. The intranasal fluid delivery device of claim 1 or 2, wherein the fluid delivery orifice of the dispensing tip is configured to be positioned within about 1 mm to about 25 mm from the anterior entry to the olfactory region.

25. The intranasal fluid delivery device of claim 1 or 2, wherein the dispensing tip comprises a hydrophilic coating applied to an outer surface of the dispensing tip.

26. The intranasal fluid delivery device of claim 25, wherein the hydrophilic coating is activated by contact with a hydrating medium.

27. The intranasal fluid delivery device of claim 26, wherein the hydrating medium is water, a gel, a lubricating gel, a viscous liquid, a vapor, or any combination thereof.

28. The intranasal fluid delivery device of claim 27, wherein the water is water vapor.

29. The intranasal fluid delivery device of claim 26, wherein activating the hydrophilic coating reduces a surface friction of the hydrophilic coating.

30. The intranasal fluid delivery device of claim 1 or 2, wherein the fluid comprises a pharmaceutical agent or a medicament.

31. The intranasal fluid delivery device of claim 30, wherein the fluid comprises ketamine or insulin.

32. The intranasal fluid delivery device of claim 30, wherein the pharmaceutical agent or the medicament is configured to be transported to the central nervous system (CNS) from the olfactory region and at least partly through olfactory neuronal pathways.

33. The intranasal fluid delivery device of claim 1 or 2, wherein the fluid comprises vitamins, fragrance, saline or non-pharmaceutical agents.

34. The intranasal fluid delivery device of claim 1 or 2, wherein the surface of a subject's nasal cavity comprises anatomical features of a nasal cavity of the subject.

35. The intranasal fluid delivery device of claim 34, wherein the anatomical features comprise nasal turbinates, a nasal valve, or combinations thereof.

36. The intranasal fluid delivery device of claim 34, wherein the anatomical features comprise a septum.

37. The intranasal fluid delivery device of claim 34, wherein the anatomical features comprise an anterior aspect of the nasal passage.

38. The intranasal fluid delivery device of claim 1 or 2, wherein the dispensing tip has an elliptical cross section.

39. The intranasal fluid delivery device of claim 1 or 2, wherein the dispensing tip has a distal portion and a proximal portion, and wherein a first center axis of the distal portion and a second center axis of the proximal portion are non-colinear.

40. The intranasal fluid delivery device of claim 1 or 2, wherein the dispensing tip comprises an off-center drug dispensing channel.

41. The intranasal fluid delivery device of claim 1 or 2, wherein the dispensing tip comprises a protruding element.

42. The intranasal fluid delivery device of claim 1 or 2, wherein the dispensing tip comprises an inflatable balloon surrounding at least a part of a distal portion of the dispensing tip.

43. The intranasal fluid delivery device of claim 42, wherein the inflatable balloon further surrounds at least a part of a proximal portion of the dispensing tip.

44. The intranasal fluid delivery device of claim 1 or 2, wherein a distal portion of the dispensing tip is curved.

45. The intranasal fluid delivery device of claim 1 or 2, wherein the dispensing tip has a perforation.

46. The intranasal fluid delivery device of claim 45, wherein the dispensing tip has a perforation on a distal portion of the dispensing tip.

47. The intranasal fluid delivery device of claim 45, wherein the perforation is on a single side of the dispensing tip.

48. The intranasal fluid delivery device of claim 1 or 2, wherein a distal portion of the dispensing tip has a spiral shape.

49. The intranasal fluid delivery device of claim 48, wherein exerting the pressure on fluid located within the dispensing tip enables an unraveling of the spiral shape.

50. The intranasal fluid delivery device of claim 1, wherein the shot chamber is removable.

51. The intranasal fluid delivery device of claim 1 or 2, further comprising a damping mechanism configured to generate a controlled velocity profile of the fluid delivered from the dispensing tip.

52. The intranasal fluid delivery device of any one of claim 1, 2, or 51 wherein the fluid is delivered from the dispensing tip with a velocity of about 0.5 m/s to about 15 m/s.

53. The intranasal fluid delivery device of any one of claim 1, 2, or 51 wherein the fluid is delivered from the dispensing tip with a velocity of about 1.5 m/s to about 9 m/s.

54. The intranasal fluid delivery device of claim 51, wherein the damping mechanism comprises at least one of a magnet, a spring, a viscous dampener, a sealed chamber with an airflow restriction, a container of compressed gas, a valve, a motor, an elastomeric chamber, a flow restriction device, and a configuration of the plunger and shot chamber.

55. The intranasal fluid delivery device of claim 54, wherein the damping mechanism comprises a flow restriction fluidically coupling the shot chamber and the dispensing tip.

56. The intranasal fluid delivery device of claim 54, wherein the flow restriction is a constriction between the shot chamber and the dispensing tip.

57. The intranasal fluid delivery device of claim 54, wherein the flow restriction is a constriction within the dispensing tip.

58. The intranasal fluid delivery device of claim 54, wherein the flow restriction is a porous body.

59. The intranasal fluid delivery device of claim 58, wherein the porous body comprises an open cell pore, a closed cell pore, or any combination thereof.

60. The intranasal fluid delivery device of claim 58, wherein the porous body is formed of metal, ceramic, plastic, wood, or any combination thereof.

61. The intranasal fluid delivery device of 54, wherein the flow restriction is an orifice plate within the dispensing tip, between the shot chamber and the dispensing tip, or both.

62. The intranasal fluid delivery device of claim 54, wherein the flow restriction is an orifice within the dispensing tip, between the shot chamber and the dispensing tip, or both.

63. The intranasal fluid delivery device of claim 54, wherein the flow restriction comprises flexible washers within the dispensing tip, between the shot chamber and the dispensing tip, or both.

64. The intranasal fluid delivery device of claim 1, further comprising a secondary chamber, wherein the secondary chamber comprises a second plunger at one end configured to drive the fluid out the dispensing tip.

65. The intranasal fluid delivery device of claim 1 or 64, wherein a fluidic pathway between the shot chamber and dispensing tip comprises a one-way valve configured to prevent backflow of the fluid into the shot chamber.

66. The intranasal fluid delivery device of claim 64 or 65, wherein the second plunger is further configured to drive a secondary fluid out of the dispensing tip.

67. The intranasal fluid delivery device of claim 66, wherein the secondary fluid is a gas.

68. The intranasal fluid delivery of claim 1, further comprising an actuator operatively coupled to the plunger and configured to move the plunger, thereby enabling the plunger to drive the fluid from the shot chamber.

69. The intranasal fluid delivery of claim 68, wherein the actuator is operatively coupled to a push rod, such that when a user or the subject engages the actuator, the actuator enables the push rod to push against the plunger, thereby causing the plunger to move.

70. The intranasal fluid delivery of claim 1, further comprising:
a. a hollow needle coupled to the dispensing tip; and
b. a diaphragm disposed between the shot chamber and the hollow needle, the diaphragm providing a fluidic barrier between the shot chamber and dispensing tip, wherein the hollow needle is configured to puncture the diaphragm so as to provide fluidic communication between the shot chamber and dispensing tip.

71. The intranasal fluid delivery device of claim 70, wherein the hollow needle is configured to be manually pushed towards the shot chamber by a user or subject so as to puncture the diaphragm.

72. The intranasal fluid delivery device of claim 71, further comprising an actuator operatively coupled to a push rod moveable toward the plunger, such that the actuator is configured to move the plunger, thereby enabling the plunger to drive the fluid from the shot chamber to the dispensing tip via the hollow needle.

73. The intranasal fluid delivery device of claim 72, configured such that when a user or the subject engages the actuator, the actuator enables the push rod to push against the plunger, thereby causing the plunger to move.

74. The intranasal fluid delivery device of claim 70, further comprising an actuator configured such that when a user or the subject engages the actuator, the actuator enables a push rod to push against the plunger, causing the shot chamber to move toward the hollow needle, such that the hollow needle punctures the diaphragm.

75. The intranasal fluid delivery device of claim 74, wherein the actuator is configured to move the plunger via the push rod, thereby enabling the plunger to drive the fluid from the shot chamber.

76. The intranasal fluid delivery device of claim 75, wherein a single actuation by the user of the actuator enables the shot chamber to move forward so as to puncture the diaphragm, and subsequently eject the fluid within the shot chamber via the plunger being pushed by the push rod.

77. The intranasal fluid delivery device of any one of claim 68-69 or 72-75, wherein the actuator comprises a locking mechanism, and wherein user or subject engagement of the actuator releases the locking mechanism, allowing the push rod to push against the plunger.

78. The intranasal fluid delivery device of claim 77, wherein the locking mechanism comprises one or both of:
a. one or more tabs comprising a lock material, configured such that the locking mechanism is released by the user breaking the lock material; and
b. one or more pivotable tabs, configured such that the locking mechanism is released by the user pivoting the pivotable tabs.

79. The intranasal fluid delivery device of claim 77 or 78, further comprising a spring in alignment with the push rod, wherein the locking mechanism is configured to maintain the spring under a pressure condition, wherein releasing the locking mechanism releases the spring from the pressure condition, causing the push rod to push against the plunger.

80. The intranasal fluid delivery device of claim 79, wherein the spring is a variable pitch spring.

81. The intranasal fluid delivery device of any one of claims 77 to 79, wherein the locking mechanism comprises one or more tabs that break off to release the push rod, such that the device is useable only once.

82. The intranasal fluid delivery device of any one of claims 1 or 68 to 81, wherein the device is configured to maintain the fluid within the shot chamber pressurized prior to being ejected through the dispensing tip.

83. The intranasal fluid delivery device of any one of claims 68 to 81, comprising a cartridge configured for containing, or containing, a pharmaceutical fluid, wherein the cartridge comprises the shot chamber, the diaphragm, and the plunger.

84. The intranasal fluid delivery device of any one claims 69 or 72 to 81, further comprising a stopping mechanism configured to limit a travel distance of the push rod.

85. The intranasal fluid delivery device of any one of claims 79 to 84, further comprising a cocking mechanism configured to be activated by the user, wherein the intranasal fluid delivery device is configured such that when the user activates the cocking mechanism, pressure is applied to the spring and the spring is thereby placed under the pressure condition.

86. The intranasal fluid delivery device of any one of claims 68-69 or 72 to 85, further comprising a damping mechanism configured to generate a controlled velocity profile of the fluid delivered from the dispensing tip, wherein the damping mechanism comprises an actuator restriction coupled to the actuator.

87. The intranasal fluid delivery device of claim 86, wherein the actuator restriction comprises a porous cavity.

88. The intranasal fluid delivery device of 87, wherein the porous cavity comprises an open cell pore, a closed cell pore, or any combination thereof.

89. The intranasal fluid delivery device of claim 88, wherein the porous cavity is formed of metal, ceramic, plastic, wood, or any combination thereof.

90. The intranasal fluid delivery device of any one of claims 68-69 or 72 to 89, further comprising a secondary chamber, wherein the secondary chamber comprises a second plunger at one end and a second actuator connected to a second push rod moveable toward the dispensing tip.

91. The intranasal fluid delivery device of any one claims 72-90, wherein the hollow needle comprises a one-way valve configured to prevent backflow.

92. The intranasal fluid delivery device of claim 90, wherein the second plunger and the second actuator are configured to drive the fluid out of the dispensing tip.

93. The intranasal fluid delivery device of claim 92, wherein the second plunger and the second actuator are further configured to drive a secondary fluid out of the dispensing tip.

94. The intranasal fluid delivery device of claim 93, wherein the secondary fluid is a gas.

95. The intranasal fluid delivery device of any one of claims 90 to 94, further comprising a second needle for providing fluid communication between the shot chamber and the secondary chamber.

96. The intranasal fluid delivery device of any one claims 90 to 95, wherein the second actuator is configured to control the flow rate of the fluid out of the dispensing tip.

97. The intranasal fluid delivery device of any one of the preceding claims, wherein the spring type, dampener type, shot chamber dimensions, dispensing tip length, or any combinations thereof is selected based on the fluid characteristics, therapeutic requirements, surface of a subject's nasal cavity, or combinations thereof.

98. A method for delivering a fluid to an olfactory region of a subject, the method comprising:
a. inserting a compliant dispensing tip into the subject's nasal cavity, wherein the dispensing tip comprises a flexible nib configured to comply with or conform to a surface of the subject's nasal cavity, thereby enabling a fluid delivery orifice of the dispensing tip to be directed towards the olfactory region of the subject; and
b. ejecting the fluid from the fluid delivery orifice of the compliant dispensing tip to deliver the fluid to the olfactory region of the subject.

99. The method of claim 98, wherein the dispensing tip is configured to deliver the fluid as a liquid jet or a liquid stream.

100. The method of claim 99, wherein the liquid jet is a laminar flow or the liquid stream is a laminar flow.

101. The method of claim 98, wherein the dispensing tip is configured to deliver the fluid as a spray, mist, or aerosol.

102. The method of claim 98, wherein the fluid comprises a powder.

103. The method of claim 98, wherein the dispensing tip comprises an atomizer.

104. The method of claim 98, wherein the dispensing tip comprises a cannula.

105. The method of claim 98, wherein the dispensing tip is tubular.

106. The method of claim 105, wherein the dispensing tip has an inner diameter from about 0.3 mm to about 1.5 mm.

107. The method of claim 98, wherein the flexible nib comprises a polymer.

108. The method of claim 98, wherein the flexible nib comprises thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene. comprises polytetrafluoroethylene (PTFE), or any combinations thereof.

109. The method of claim 98, wherein the dispensing tip comprises a distal portion having a first rigidity and a proximal portion having a second rigidity, and wherein the first rigidity is less than the second rigidity.

110. The method of claim 109, wherein the distal portion comprising a first rigidity comprises a portion of the dispensing tip that is about 1 mm to about 15 mm from the fluid delivery orifice.

111. The method of claim 98, wherein the dispensing tip comprises a distal portion that is softer than a proximal portion.

112. The method of claim 111, wherein the distal portion that is softer than a proximal portion comprises a portion of the dispensing tip that is about 1 mm to about 15 mm from the fluid delivery orifice.

113. The method of claim 98, wherein the dispensing tip has a distal portion having a first outer diameter and a proximal portion having a second outer diameter, and wherein the first outer diameter is less than the second outer diameter.

114. The method of claim 98, wherein the dispensing tip has a distal portion having a first outer diameter, a proximal portion having a second outer diameter, and wherein the first outer diameter is greater than the second outer diameter.

115. The method of claim 98, wherein the dispensing tip further comprises a nose cushion to limit over-insertion of the dispensing tip within the nasal cavity, wherein the nose cushion configured to provide subject comfort

116. The method of claim 98, wherein the dispensing tip is configured to be inserted to an insertion depth of about 10 mm to about 90 mm within the subject's nasal cavity.

117. The method of claim 98, wherein the fluid delivery orifice of the dispensing tip is configured to be positioned at or near the anterior entry to the olfactory region.

118. The method of claim 98, wherein the fluid delivery orifice of the dispensing tip is configured to be positioned within about 1 mm to about 25 mm from the anterior entry to the olfactory region.

119. The method of claim 98, wherein the dispensing tip comprises a hydrophilic coating applied to an outer surface of the dispensing tip.

120. The method of claim 98, wherein the fluid comprises a pharmaceutical agent or a medicament.

121. The method of claim 120, wherein the fluid comprises ketamine or insulin.

122. The method of claim 120, wherein the pharmaceutical agent or the medicament is configured to be absorbed by the central nervous system (CNS) through the olfactory region.

123. The method of claim 98, wherein the fluid comprises vitamins, fragrance, saline or non-pharmaceutical agents.

124. The method of claim 98, wherein the surface of a subject's nasal cavity comprises anatomical features of a nasal cavity of the subject.

125. The method of claim 124, wherein the anatomical features comprise nasal turbinates, a nasal valve, or combinations thereof.

126. The method of claim 124, wherein the anatomical features comprise a septum.

127. The method of claim 124, wherein the anatomical features comprise an anterior aspect of the nasal passage.

128. The method of claim 98, wherein the dispensing tip has an elliptical cross section.

129. The method of claim 98, wherein the dispensing tip has a distal portion and a proximal portion, and wherein a first center axis of the distal portion and a second center axis of the proximal portion are non-colinear.

130. The method of claim 98, wherein the dispensing tip comprises an off-center drug dispensing channel.

131. The method of claim 98, wherein the dispensing tip comprises a protruding element.

132. The method of claim 98, wherein the dispensing tip comprises an inflatable balloon surrounding at least a part of a distal portion of the dispensing tip.

133. The method of claim 132, wherein the inflatable balloon further surrounds at least a part of a proximal portion of the dispensing tip.

134. The method of claim 98, wherein a distal portion of the dispensing tip is curved.

135. The method of claim 98, wherein the dispensing tip has a perforation.

136. The method of claim 135, wherein the dispensing tip has a perforation on a distal portion of the dispensing tip.

137. The method of claim 135, wherein the perforation is on a single side of the dispensing tip.

138. The method of claim 98, wherein a distal portion of the dispensing tip has a spiral shape.

139. The method of claim 138, wherein exerting the pressure on fluid located within the dispensing tip enables an unraveling of the spiral shape.
